SEQUENCE LISTING (378 sequences)

SEQ ID NO: 1 - DNA, 88 bp, Artificial Sequence
  Synthetic primer
ttgaaaatcaccccactgcaaactcctccccctgctagaaacctcacattgaaatgctgtaaatgacgtgggccccgagtgcaatcgc

SEQ ID NO: 2 - DNA, 455 bp, Mus musculus
cacatgactttgaactatgctgtctctatggattaataggacagaacctttttcaaatacaatggatatgaataagagaagacagcagtgtaaatgctgaagatcatctctttcacacatgtgctgctggatagtcctggtattgctgcaccaaaccaacctaactcaactgctaaggtcaaagtgtctgatttcttctctgctacagaagaggatgttgtcaaccatgagtcacatagcctctggctttctagaagtacaagaagtctctatataaagatggaagttcctgactaggatgttggagcaggcaggaaggaggccactggtgattttgcccagagaaaagcttcagcatgcctagccttcctttgctgttgcttctctgggctgctagctcatacagtttccctgtgtttcacaacggagaccggcaaaatgtggagacagtctgg

SEQ ID NO: 3 - DNA, 500 bp, Homo sapiens
tatgttcagaagagacaaaatgacatacacctagaacgctctaccatgatgtcactgattttacctaaaaccacttttggaatccataaatggggcaagggaaatggattcccctcccccaccccagtttatatggtataaattccatgtgaatagagaatagttgagttttttgtatatcctcatttcccacatgaacattttttatattgtctacttcccatttcaaatttgaaaagcagtctctgtaagggtggcgttgtcatcagaaagtaacaataataaatatagaaagtgtataaaacagtagaaacaaggttgactttattccaaacataaaacacttattttgtgttagaagagttatcccttgcctatccagggtgacaccagtgactgcacatgtgttcttgagctgcttttcctccggcaaagactcatgtctcctgtctctttctgtcttgaaaggatatgtttgtcactgaagctgctctctggga

SEQ ID NO: 4 - DNA, 440 bp, Rattus norvegicus
gaactatgctgcatctatggatttaataggccagaacctttttcaaataaaatggacatgaatcagagaaaacaagtttagtttctgaaggccatgcgtttcacacatgtgctgcggatattaacctgggattgctgcaccaaaccaacctaaatcatgtgctagggtcaaagtgtctgatttcttccctgctactgaagaggatgttgtcaaccatgagtcacgtagcctctggttttgtagaagtagaaatctctataaaggtggaggttcttggacaggatgttggagcaggcaggaagaaggccactggtgatcttgcgcagagagaagcttcagcatgcttagccttcctttgctgttgcttctcttggctaccagctcatacagtttccccgtgtttcaggacggagcccggcagaatgtggaaacagtctggg

SEQ ID NO: 5 - RNA, 21 nt, Artificial Sequence
  Synthetic primer
ucugcuacagaagaggauguu

SEQ ID NO: 6 - RNA, 21 nt, Artificial Sequence
  Synthetic primer
gcuacagaagaggauguuguc

SEQ ID NO: 7 - RNA, 21 nt, Artificial Sequence
  Synthetic primer
acagaagaggauguugucaac

SEQ ID NO: 8 - RNA, 21 nt, Artificial Sequence
  Synthetic primer
gaagaggauguugucaaccau

SEQ ID NO: 9 - RNA, 21 nt, Artificial Sequence
  Synthetic primer
agaggauguugucaaccauga

SEQ ID NO: 10 - RNA, 21 nt, Artificial Sequence
  Synthetic primer
gaggauguugucaaccaugag

SEQ ID NO: 11 - DNA, 21 bp, Artificial Sequence
  Synthetic primer
aggauguugucaaccaugagt

SEQ ID NO: 12 - RNA, 21 nt, Artificial Sequence
  Synthetic primer
auguugucaaccaugagucac

SEQ ID NO: 13 - RNA, 21 nt, Artificial Sequence
  Synthetic primer
guugucaaccaugagucacau

SEQ ID NO: 14 - RNA, 19 nt, Artificial Sequence
  Synthetic primer
gguuaaaagagacgauguc

SEQ ID NO: 15 - RNA, 19 nt, Artificial Sequence
  Synthetic primer
gacaucgucucuuuuaacc

SEQ ID NO: 16 - RNA, 19 nt, Artificial Sequence
  Synthetic primer
uaaaagagacgaugucaca

SEQ ID NO: 17 - RNA, 19 nt, Artificial Sequence
  Synthetic primer
ugugacaucgucucuuuua

SEQ ID NO: 18 - RNA, 19 nt, Artificial Sequence
  Synthetic primer
aagagacgaugucacaucg

SEQ ID NO: 19 - RNA, 19 nt, Artificial Sequence
  Synthetic primer
cgaugugacaucgucucuu

SEQ ID NO: 20 - RNA, 19 nt, Artificial Sequence
  Synthetic primer
agacgaugucacaucggcc

SEQ ID NO: 21 - RNA, 19 nt, Artificial Sequence
  Synthetic primer
ggccgaugugacaucgucu

SEQ ID NO: 22 - RNA, 19 nt, Artificial Sequence
  Synthetic primer
cgaugucacaucggccguu

SEQ ID NO: 23 - RNA, 19 nt, Artificial Sequence
  Synthetic primer
aacggccgaugugacaucg

SEQ ID NO: 24 - RNA, 19 nt, Artificial Sequence
  Synthetic primer
gaugucacaucggccguuc

SEQ ID NO: 25 - RNA, 19 nt, Artificial Sequence
  Synthetic primer
gaacggccgaugugacauc

SEQ ID NO: 26 - RNA, 19 nt, Artificial Sequence
  Synthetic primer
augucacaucggccguucc

SEQ ID NO: 27 - RNA, 19 nt, Artificial Sequence
  Synthetic primer
ggaacggccgaugugacau

SEQ ID NO: 28 - RNA, 19 nt, Artificial Sequence
  Synthetic primer
ugucacaucggccguucca

SEQ ID NO: 29 - RNA, 19 nt, Artificial Sequence
  Synthetic primer
uggaacggccgaugugaca

SEQ ID NO: 30 - RNA, 19 nt, Artificial Sequence
  Synthetic primer
gucacaucggccguuccaa

SEQ ID NO: 31 - RNA, 19 nt, Artificial Sequence
  Synthetic primer
uuggaacggccgaugugac

SEQ ID NO: 32 - RNA, 19 nt, Artificial Sequence
  Synthetic primer
ucacaucggccguuccaag

SEQ ID NO: 33 - RNA, 19 nt, Artificial Sequence
  Synthetic primer
cuuggaacggccgauguga

SEQ ID NO: 34 - RNA, 19 nt, Artificial Sequence
  Synthetic primer
cacaucggccguuccaagc

SEQ ID NO: 35 - RNA, 19 nt, Artificial Sequence
  Synthetic primer
gcuuggaacggccgaugug

SEQ ID NO: 36 - RNA, 19 nt, Artificial Sequence
  Synthetic primer
acaucggccguuccaagcu

SEQ ID NO: 37 - RNA, 19 nt, Artificial Sequence
  Synthetic primer
agcuuggaacggccgaugu

SEQ ID NO: 38 - RNA, 19 nt, Artificial Sequence
  Synthetic primer
caucggccguuccaagcuc

SEQ ID NO: 39 - RNA, 19 nt, Artificial Sequence
  Synthetic primer
gagcuuggaacggccgaug

SEQ ID NO: 40 - RNA, 19 nt, Artificial Sequence
  Synthetic primer
aucggccguuccaagcucc

SEQ ID NO: 41 - RNA, 19 nt, Artificial Sequence
  Synthetic primer
ggagcuuggaacggccgau

SEQ ID NO: 42 - RNA, 19 nt, Artificial Sequence
  Synthetic primer
ucggccguuccaagcuccu

SEQ ID NO: 43 - RNA, 19 nt, Artificial Sequence
  Synthetic primer
aggagcuuggaacggccga

SEQ ID NO: 44 - RNA, 19 nt, Artificial Sequence
  Synthetic primer
cggccguuccaagcuccuc

SEQ ID NO: 45 - RNA, 19 nt, Artificial Sequence
  Synthetic primer
gaggagcuuggaacggccg

SEQ ID NO: 46 - RNA, 19 nt, Artificial Sequence
  Synthetic primer
ggccguuccaagcuccucc

SEQ ID NO: 47 - RNA, 19 nt, Artificial Sequence
  Synthetic primer
ggaggagcuuggaacggcc

SEQ ID NO: 48 - RNA, 19 nt, Artificial Sequence
  Synthetic primer
gccguuccaagcuccuccc

SEQ ID NO: 49 - RNA, 19 nt, Artificial Sequence
  Synthetic primer
gggaggagcuuggaacggc

SEQ ID NO: 50 - RNA, 19 nt, Artificial Sequence
  Synthetic primer
ccguuccaagcuccucccc

SEQ ID NO: 51 - RNA, 19 nt, Artificial Sequence
  Synthetic primer
ggggaggagcuuggaacgg

SEQ ID NO: 52 - RNA, 19 nt, Artificial Sequence
  Synthetic primer
cguuccaagcuccuccccg

SEQ ID NO: 53 - RNA, 19 nt, Artificial Sequence
  Synthetic primer
cggggaggagcuuggaacg

SEQ ID NO: 54 - RNA, 19 nt, Artificial Sequence
  Synthetic primer
guuccaagcuccuccccgc

SEQ ID NO: 55 - RNA, 19 nt, Artificial Sequence
  Synthetic primer
gcggggaggagcuuggaac

SEQ ID NO: 56 - RNA, 19 nt, Artificial Sequence
  Synthetic primer
uccaagcuccuccccgcuc

SEQ ID NO: 57 - RNA, 19 nt, Artificial Sequence
  Synthetic primer
gagcggggaggagcuugga

SEQ ID NO: 58 - RNA, 19 nt, Artificial Sequence
  Synthetic primer
aagcuccuccccgcucagu

SEQ ID NO: 59 - RNA, 19 nt, Artificial Sequence
  Synthetic primer
acugagcggggaggagcuu

SEQ ID NO: 60 - RNA, 19 nt, Artificial Sequence
  Synthetic primer
cuccuccccgcucagugag

SEQ ID NO: 61 - RNA, 19 nt, Artificial Sequence
  Synthetic primer
cucacugagcggggaggag

SEQ ID NO: 62 - RNA, 19 nt, Artificial Sequence
  Synthetic primer
cuccccgcucagugaggug

SEQ ID NO: 63 - RNA, 19 nt, Artificial Sequence
  Synthetic primer
caccucacugagcggggag

SEQ ID NO: 64 - RNA, 19 nt, Artificial Sequence
  Synthetic primer
cccgcucagugaggugaag

SEQ ID NO: 65 - RNA, 19 nt, Artificial Sequence
  Synthetic primer
cuucaccucacugagcggg

SEQ ID NO: 66 - RNA, 19 nt, Artificial Sequence
  Synthetic primer
gcucagugaggugaagauu

SEQ ID NO: 67 - RNA, 19 nt, Artificial Sequence
  Synthetic primer
aaucuucaccucacugagc

SEQ ID NO: 68 - RNA, 19 nt, Artificial Sequence
  Synthetic primer
cagugaggugaagauuuuu

SEQ ID NO: 69 - RNA, 19 nt, Artificial Sequence
  Synthetic primer
aaaaaucuucaccucacug

SEQ ID NO: 70 - RNA, 19 nt, Artificial Sequence
  Synthetic primer
ugaggugaagauuuuugaa

SEQ ID NO: 71 - RNA, 19 nt, Artificial Sequence
  Synthetic primer
uucaaaaaucuucaccuca

SEQ ID NO: 72 - RNA, 19 nt, Artificial Sequence
  Synthetic primer
ggugaagauuuuugaaaau

SEQ ID NO: 73 - RNA, 19 nt, Artificial Sequence
  Synthetic primer
auuuucaaaaaucuucacc

SEQ ID NO: 74 - RNA, 19 nt, Artificial Sequence
  Synthetic primer
gaagauuuuugaaaaucac

SEQ ID NO: 75 - RNA, 19 nt, Artificial Sequence
  Synthetic primer
gugauuuucaaaaaucuuc

SEQ ID NO: 76 - RNA, 19 nt, Artificial Sequence
  Synthetic primer
agauuuuugaaaaucaccc

SEQ ID NO: 77 - RNA, 19 nt, Artificial Sequence
  Synthetic primer
gggugauuuucaaaaaucu

SEQ ID NO: 78 - RNA, 19 nt, Artificial Sequence
  Synthetic primer
gauuuuugaaaaucacccc

SEQ ID NO: 79 - RNA, 19 nt, Artificial Sequence
  Synthetic primer
ggggugauuuucaaaaauc

SEQ ID NO: 80 - RNA, 19 nt, Artificial Sequence
  Synthetic primer
auuuuugaaaaucacccca

SEQ ID NO: 81 - RNA, 19 nt, Artificial Sequence
  Synthetic primer
uggggugauuuucaaaaau

SEQ ID NO: 82 - RNA, 19 nt, Artificial Sequence
  Synthetic primer
uuuugaaaaucaccccauu

SEQ ID NO: 83 - RNA, 19 nt, Artificial Sequence
  Synthetic primer
aauggggugauuuucaaaa

SEQ ID NO: 84 - RNA, 19 nt, Artificial Sequence
  Synthetic primer
ugaaaaucaccccauugca

SEQ ID NO: 85 - RNA, 19 nt, Artificial Sequence
  Synthetic primer
ugcaauggggugauuuuca

SEQ ID NO: 86 - RNA, 19 nt, Artificial Sequence
  Synthetic primer
aaaucaccccauugcagac

SEQ ID NO: 87 - RNA, 19 nt, Artificial Sequence
  Synthetic primer
gucugcaauggggugauuu

SEQ ID NO: 88 - RNA, 19 nt, Artificial Sequence
  Synthetic primer
ucaccccauugcagacucc

SEQ ID NO: 89 - RNA, 19 nt, Artificial Sequence
  Synthetic primer
ggagucugcaaugggguga

SEQ ID NO: 90 - RNA, 19 nt, Artificial Sequence
  Synthetic primer
ccccauugcagacuccucc

SEQ ID NO: 91 - RNA, 19 nt, Artificial Sequence
  Synthetic primer
ggaggagucugcaaugggg

SEQ ID NO: 92 - RNA, 19 nt, Artificial Sequence
  Synthetic primer
cauugcagacuccuccccc

SEQ ID NO: 93 - RNA, 19 nt, Artificial Sequence
  Synthetic primer
gggggaggagucugcaaug

SEQ ID NO: 94 - RNA, 19 nt, Artificial Sequence
  Synthetic primer
ugcagacuccucccccgcc

SEQ ID NO: 95 - RNA, 19 nt, Artificial Sequence
  Synthetic primer
ggcgggggaggagucugca

SEQ ID NO: 96 - RNA, 19 nt, Artificial Sequence
  Synthetic primer
agacuccucccccgccugg

SEQ ID NO: 97 - RNA, 19 nt, Artificial Sequence
  Synthetic primer
ccaggcgggggaggagucu

SEQ ID NO: 98 - RNA, 19 nt, Artificial Sequence
  Synthetic primer
cuccucccccgccuggaaa

SEQ ID NO: 99 - RNA, 19 nt, Artificial Sequence
  Synthetic primer
uuuccaggcgggggaggag

SEQ ID NO: 100 - RNA, 19 nt, Artificial Sequence
  Synthetic primer
cucccccgccuggaaaccu

SEQ ID NO: 101 - RNA, 19 nt, Artificial Sequence
  Synthetic primer
agguuuccaggcgggggag

SEQ ID NO: 102 - DNA, 23 bp, Artificial Sequence
  Synthetic primer
ugcuguaaaugacgugggcdtdt

SEQ ID NO: 103 - DNA, 23 bp, Artificial Sequence
  Synthetic primer
gcccacgucauuuacagcadtdt

SEQ ID NO: 104 - DNA, 23 bp, Artificial Sequence
  Synthetic primer
ugcuguaaaugacgugggcdtdt

SEQ ID NO: 105 - DNA, 23 bp, Artificial Sequence
  Synthetic primer
gcccacgucauuuacagcadtdt

SEQ ID NO: 106 - DNA, 22 bp, Artificial Sequence
  Synthetic primer
gcuguaaaugacgugggcdtdt

SEQ ID NO: 107 - DNA, 33 bp, Artificial Sequence
  Synthetic primer
gcncncnacnguncaunununacnagcnadtdt

SEQ ID NO: 108 - DNA, 31 bp, Artificial Sequence
  Synthetic primer
ungcnungunaaaungacngungggcndtdt

SEQ ID NO: 109 - DNA, 23 bp, Artificial Sequence
  Synthetic primer
gcccacgucauuuacagcadtdt

SEQ ID NO: 110 - DNA, 31 bp, Artificial Sequence; identical to SEQ ID NO: 108
  Synthetic primer

SEQ ID NO: 111 - DNA, 33 bp, Artificial Sequence; identical to SEQ ID NO: 107
  Synthetic primer

SEQ ID NO: 112 - DNA, 23 bp, Artificial Sequence
  Synthetic primer
aaacuccucccccugcuagdtdt

SEQ ID NO: 113 - DNA, 23 bp, Artificial Sequence
  Synthetic primer
cuagcagggggaggaguuudtdt

SEQ ID NO: 114 - DNA, 23 bp, Artificial Sequence
  Synthetic primer
aaacuccucccccugcuagdtdt

SEQ ID NO: 115 - DNA, 23 bp, Artificial Sequence
  Synthetic primer
cuagcagggggaggaguuudtdt

SEQ ID NO: 116 - DNA, 23 bp, Artificial Sequence
  Synthetic primer
aaacuccucccccugcuagdtdt

SEQ ID NO: 117 - DNA, 28 bp, Artificial Sequence
  Synthetic primer
cunagcnagggggaggagununundtdt

SEQ ID NO: 118 - DNA, 36 bp, Artificial Sequence
  Synthetic primer
aaacnuncncnuncncncncncnungcnunagdtdt

SEQ ID NO: 119 - DNA, 23 bp, Artificial Sequence
  Synthetic primer
cuagcagggggaggaguuudtdt

SEQ ID NO: 120 - DNA, 36 bp, Artificial Sequence; identical to SEQ ID NO: 118
  Synthetic primer

SEQ ID NO: 121 - DNA, 28 bp, Artificial Sequence
  Synthetic primer
cunagcnagggggaggagununundtdt

SEQ ID NO: 122 - DNA, 23 bp, Artificial Sequence
  Synthetic primer
uuccagugcucugauggaadtdt

SEQ ID NO: 123 - DNA, 23 bp, Artificial Sequence
  Synthetic primer
uuccaucagagcacuggaadtdt

SEQ ID NO: 124 - DNA, 23 bp, Artificial Sequence
  Synthetic primer
uuccagugcucugauggaadtdt

SEQ ID NO: 125 - DNA, 23 bp, Artificial Sequence
  Synthetic primer
uuccaucagagcacuggaadtdt

SEQ ID NO: 126 - DNA, 23 bp, Artificial Sequence
  Synthetic primer
uuccagugcucugauggaadtdt

SEQ ID NO: 127 - DNA, 32 bp, Artificial Sequence
  Synthetic primer
ununcncnauncnagagcnacnunggaadtdt

SEQ ID NO: 128 - DNA, 33 bp, Artificial Sequence
  Synthetic primer
ununcncnagungcnuncnungaunggaadtdt

SEQ ID NO: 129 - DNA, 23 bp, Artificial Sequence
  Synthetic primer
uuccaucagagcacuggaadtdt

SEQ ID NO: 130 - DNA, 33 bp, Artificial Sequence; identical to SEQ ID NO: 128
  Synthetic primer

SEQ ID NO: 131 - DNA, 32 bp, Artificial Sequence; identical to SEQ ID NO: 127
  Synthetic primer

SEQ ID NO: 132 - DNA, 23 bp, Artificial Sequence
  Synthetic primer
aaacuccucccccugcuagdtdt

SEQ ID NO: 133 - DNA, 28 bp, Artificial Sequence
  Synthetic primer
cunagcnagggggaggagununundtdt

SEQ ID NO: 134 - DNA, 23 bp, Artificial Sequence
  Synthetic primer
aaacuccucccccugcuagdtdt

SEQ ID NO: 135 - DNA, 23 bp, Artificial Sequence
  Synthetic primer
cuagcagggggaggaguuudtdt

SEQ ID NO: 136 - DNA, 36 bp, Artificial Sequence; identical to SEQ ID NO: 118
  Synthetic primer

SEQ ID NO: 137 - DNA, 23 bp, Artificial Sequence
  Synthetic primer
cuagcagggggaggaguuudtdt

SEQ ID NO: 138 - DNA, 23 bp, Artificial Sequence
  Synthetic primer
ugcuguaaaugacgugggcdtdt

SEQ ID NO: 139 - DNA, 23 bp, Artificial Sequence
  Synthetic primer
gcccacgucauuuacagcadtdt

SEQ ID NO: 140 - DNA, 23 bp, Artificial Sequence
  Synthetic primer
ugcuguaaaugacgugggcdtdt

SEQ ID NO: 141 - DNA, 23 bp, Artificial Sequence
  Synthetic primer
gcccacgucauuuacagcadtdt

SEQ ID NO: 142 - DNA, 23 bp, Artificial Sequence
  Synthetic primer
ugcuguaaaugacgugggcdtdt

SEQ ID NO: 143 - DNA, 33 bp, Artificial Sequence; identical to SEQ ID NO: 107
  Synthetic primer

SEQ ID NO: 144 - DNA, 31 bp, Artificial Sequence; identical to SEQ ID NO: 108
  Synthetic primer

SEQ ID NO: 145 - DNA, 23 bp, Artificial Sequence
  Synthetic primer
gcccacgucauuuacagcadtdt

SEQ ID NO: 146 - DNA, 23 bp, Artificial Sequence
  Synthetic primer
ugcuguaaaugacgugggcdtdt

SEQ ID NO: 147 - DNA, 23 bp, Artificial Sequence
  Synthetic primer
gcccacgucauuuacagcadtdt

SEQ ID NO: 148 - DNA, 24 bp, Artificial Sequence
  Synthetic primer
ugcugunaaaugacgugggcdtdt

SEQ ID NO: 149 - DNA, 27 bp, Artificial Sequence
  Synthetic primer
gcccacgucnauuunacnagcnadtdt

SEQ ID NO: 150 - DNA, 21 bp, Artificial Sequence
  Synthetic primer
gaaaugcuguaaaugacgutt

SEQ ID NO: 151 - DNA, 21 bp, Artificial Sequence
  Synthetic primer
acgucauuuacagcauuuctt

SEQ ID NO: 152 - DNA, 21 bp, Artificial Sequence
  Synthetic primer
gaaaugcuguaaaugacgutt

SEQ ID NO: 153 - DNA, 21 bp, Artificial Sequence
  Synthetic primer
acgucauuuacagcauuuctt

SEQ ID NO: 154 - DNA, 26 bp, Artificial Sequence
  Synthetic primer
gaaaungcungunaaaungacguntt

SEQ ID NO: 155 - DNA, 28 bp, Artificial Sequence
  Synthetic primer
acguncaunununacagcaunununctt

SEQ ID NO: 156 - DNA, 21 bp, Artificial Sequence
  Synthetic primer
gaaaugcuguaaaugacgutt

SEQ ID NO: 157 - DNA, 21 bp, Artificial Sequence
  Synthetic primer
acgucauuuacagcauuuctt

SEQ ID NO: 158 - RNA, 19 nt, Artificial Sequence
  Synthetic primer
auuugaaaaucaccccacu

SEQ ID NO: 159 - RNA, 19 nt, Artificial Sequence
  Synthetic primer
aguggggugauuuucaaau

SEQ ID NO: 160 - RNA, 19 nt, Artificial Sequence
  Synthetic primer
uuugaaaaucaccccacug

SEQ ID NO: 161 - RNA, 19 nt, Artificial Sequence
  Synthetic primer
caguggggugauuuucaaa

SEQ ID NO: 162 - RNA, 19 nt, Artificial Sequence
  Synthetic primer
uugaaaaucaccccacugc

SEQ ID NO: 163 - RNA, 19 nt, Artificial Sequence
  Synthetic primer
gcaguggggugauuuucaa

SEQ ID NO: 164 - RNA, 19 nt, Artificial Sequence
  Synthetic primer
ugaaaaucaccccacugca

SEQ ID NO: 165 - RNA, 19 nt, Artificial Sequence
  Synthetic primer
ugcaguggggugauuuuca

SEQ ID NO: 166 - RNA, 19 nt, Artificial Sequence
  Synthetic primer
gaaaaucaccccacugcaa

SEQ ID NO: 167 - RNA, 19 nt, Artificial Sequence
  Synthetic primer
uugcaguggggugauuuuc

SEQ ID NO: 168 - RNA, 19 nt, Artificial Sequence
  Synthetic primer
aaaucaccccacugcaaac

SEQ ID NO: 169 - RNA, 19 nt, Artificial Sequence
  Synthetic primer
guuugcaguggggugauuu

SEQ ID NO: 170 - RNA, 19 nt, Artificial Sequence
  Synthetic primer
aaucaccccacugcaaacu

SEQ ID NO: 171 - RNA, 19 nt, Artificial Sequence
  Synthetic primer
aguuugcaguggggugauu

SEQ ID NO: 172 - RNA, 19 nt, Artificial Sequence
  Synthetic primer
aucaccccacugcaaacuc

SEQ ID NO: 173 - RNA, 19 nt, Artificial Sequence
  Synthetic primer
gaguuugcaguggggugau

SEQ ID NO: 174 - RNA, 19 nt, Artificial Sequence
  Synthetic primer
ucaccccacugcaaacucc

SEQ ID NO: 175 - RNA, 19 nt, Artificial Sequence
  Synthetic primer
ggaguuugcagugggguga

SEQ ID NO: 176 - RNA, 19 nt, Artificial Sequence
  Synthetic primer
caccccacugcaaacuccu

SEQ ID NO: 177 - RNA, 19 nt, Artificial Sequence
  Synthetic primer
aggaguuugcaguggggug

SEQ ID NO: 178 - RNA, 19 nt, Artificial Sequence
  Synthetic primer
accccacugcaaacuccuc

SEQ ID NO: 179 - RNA, 19 nt, Artificial Sequence
  Synthetic primer
gaggaguuugcaguggggu

SEQ ID NO: 180 - RNA, 19 nt, Artificial Sequence
  Synthetic primer
cccacugcaaacuccuccc

SEQ ID NO: 181 - RNA, 19 nt, Artificial Sequence
  Synthetic primer
gggaggaguuugcaguggg

SEQ ID NO: 182 - RNA, 19 nt, Artificial Sequence
  Synthetic primer
ccacugcaaacuccucccc

SEQ ID NO: 183 - RNA, 19 nt, Artificial Sequence
  Synthetic primer
ggggaggaguuugcagugg

SEQ ID NO: 184 - RNA, 19 nt, Artificial Sequence
  Synthetic primer
acugcaaacuccucccccu

SEQ ID NO: 185 - RNA, 19 nt, Artificial Sequence
  Synthetic primer
agggggaggaguuugcagu

SEQ ID NO: 186 - RNA, 19 nt, Artificial Sequence
  Synthetic primer
cugcaaacuccucccccug

SEQ ID NO: 187 - RNA, 19 nt, Artificial Sequence
  Synthetic primer
cagggggaggaguuugcag

SEQ ID NO: 188 - RNA, 19 nt, Artificial Sequence
  Synthetic primer
ugcaaacuccucccccugc

SEQ ID NO: 189 - RNA, 19 nt, Artificial Sequence
  Synthetic primer
gcagggggaggaguuugca

SEQ ID NO: 190 - RNA, 19 nt, Artificial Sequence
  Synthetic primer
gcaaacuccucccccugcu

SEQ ID NO: 191 - RNA, 19 nt, Artificial Sequence
  Synthetic primer
agcagggggaggaguuugc

SEQ ID NO: 192 - RNA, 19 nt, Artificial Sequence
  Synthetic primer
caaacuccucccccugcua

SEQ ID NO: 193 - RNA, 19 nt, Artificial Sequence
  Synthetic primer
uagcagggggaggaguuug

SEQ ID NO: 194 - RNA, 19 nt, Artificial Sequence
  Synthetic primer
aacuccucccccugcuaga

SEQ ID NO: 195 - RNA, 19 nt, Artificial Sequence
  Synthetic primer
ucuagcagggggaggaguu

SEQ ID NO: 196 - RNA, 19 nt, Artificial Sequence
  Synthetic primer
acuccucccccugcuagaa

SEQ ID NO: 197 - RNA, 19 nt, Artificial Sequence
  Synthetic primer
uucuagcagggggaggagu

SEQ ID NO: 198 - RNA, 19 nt, Artificial Sequence
  Synthetic primer
uccucccccugcuagaaac

SEQ ID NO: 199 - RNA, 19 nt, Artificial Sequence
  Synthetic primer
guuucuagcagggggagga

SEQ ID NO: 200 - RNA, 19 nt, Artificial Sequence
  Synthetic primer
ccucccccugcuagaaacc

SEQ ID NO: 201 - RNA, 19 nt, Artificial Sequence
  Synthetic primer
gguuucuagcagggggagg

SEQ ID NO: 202 - RNA, 19 nt, Artificial Sequence
  Synthetic primer
cucccccugcuagaaaccu

SEQ ID NO: 203 - RNA, 19 nt, Artificial Sequence
  Synthetic primer
agguuucuagcagggggag

SEQ ID NO: 204 - RNA, 19 nt, Artificial Sequence
  Synthetic primer
ucccccugcuagaaaccuc

SEQ ID NO: 205 - RNA, 19 nt, Artificial Sequence
  Synthetic primer
gagguuucuagcaggggga

SEQ ID NO: 206 - RNA, 19 nt, Artificial Sequence
  Synthetic primer
cccccugcuagaaaccuca

SEQ ID NO: 207 - RNA, 19 nt, Artificial Sequence
  Synthetic primer
ugagguuucuagcaggggg

SEQ ID NO: 208 - RNA, 19 nt, Artificial Sequence
  Synthetic primer
ccccugcuagaaaccucac

SEQ ID NO: 209 - RNA, 19 nt, Artificial Sequence
  Synthetic primer
gugagguuucuagcagggg

SEQ ID NO: 210 - RNA, 19 nt, Artificial Sequence
  Synthetic primer
ucacauugaaaugcuguaa

SEQ ID NO: 211 - RNA, 19 nt, Artificial Sequence
  Synthetic primer
uuacagcauuucaauguga

SEQ ID NO: 212 - RNA, 19 nt, Artificial Sequence
  Synthetic primer
cacauugaaaugcuguaaa

SEQ ID NO: 213 - RNA, 19 nt, Artificial Sequence
  Synthetic primer
uuuacagcauuucaaugug

SEQ ID NO: 214 - RNA, 19 nt, Artificial Sequence
  Synthetic primer
acauugaaaugcuguaaau

SEQ ID NO: 215 - RNA, 19 nt, Artificial Sequence
  Synthetic primer
auuuacagcauuucaaugu

SEQ ID NO: 216 - RNA, 19 nt, Artificial Sequence
  Synthetic primer
cauugaaaugcuguaaaug

SEQ ID NO: 217 - RNA, 19 nt, Artificial Sequence
  Synthetic primer
cauuuacagcauuucaaug

SEQ ID NO: 218 - RNA, 19 nt, Artificial Sequence
  Synthetic primer
auugaaaugcuguaaauga

SEQ ID NO: 219 - RNA, 19 nt, Artificial Sequence
  Synthetic primer
ucauuuacagcauuucaau

SEQ ID NO: 220 - RNA, 19 nt, Artificial Sequence
  Synthetic primer
uugaaaugcuguaaaugac

SEQ ID NO: 221 - RNA, 19 nt, Artificial Sequence
  Synthetic primer
gucauuuacagcauuucaa

SEQ ID NO: 222 - RNA, 19 nt, Artificial Sequence
  Synthetic primer
ugaaaugcuguaaaugacg

SEQ ID NO: 223 - RNA, 19 nt, Artificial Sequence
  Synthetic primer
cgucauuuacagcauuuca

SEQ ID NO: 224 - RNA, 19 nt, Artificial Sequence
  Synthetic primer
aaaugcuguaaaugacgug

SEQ ID NO: 225 - RNA, 19 nt, Artificial Sequence
  Synthetic primer
cacgucauuuacagcauuu

SEQ ID NO: 226 - RNA, 19 nt, Artificial Sequence
  Synthetic primer
aaugcuguaaaugacgugg

SEQ ID NO: 227 - RNA, 19 nt, Artificial Sequence
  Synthetic primer
ccacgucauuuacagcauu

SEQ ID NO: 228 - RNA, 19 nt, Artificial Sequence
  Synthetic primer
augcuguaaaugacguggg

SEQ ID NO: 229 - RNA, 19 nt, Artificial Sequence
  Synthetic primer
cccacgucauuuacagcau

SEQ ID NO: 230 - RNA, 19 nt, Artificial Sequence
  Synthetic primer
gcuguaaaugacgugggcc

SEQ ID NO: 231 - RNA, 19 nt, Artificial Sequence
  Synthetic primer
ggcccacgucauuuacagc

SEQ ID NO: 232 - RNA, 19 nt, Artificial Sequence
  Synthetic primer
cuguaaaugacgugggccc

SEQ ID NO: 233 - RNA, 19 nt, Artificial Sequence
  Synthetic primer
gggcccacgucauuuacag

SEQ ID NO: 234 - RNA, 19 nt, Artificial Sequence
  Synthetic primer
guaaaugacgugggccccg

SEQ ID NO: 235 - RNA, 19 nt, Artificial Sequence
  Synthetic primer
cggggcccacgucauuuac

SEQ ID NO: 236 - RNA, 19 nt, Artificial Sequence
  Synthetic primer
uaaaugacgugggccccga

SEQ ID NO: 237 - RNA, 19 nt, Artificial Sequence
  Synthetic primer
ucggggcccacgucauuua

SEQ ID NO: 238 - RNA, 19 nt, Artificial Sequence
  Synthetic primer
aaugacgugggccccgagu

SEQ ID NO: 239 - RNA, 19 nt, Artificial Sequence
  Synthetic primer
acucggggcccacgucauu

SEQ ID NO: 240 - RNA, 19 nt, Artificial Sequence
  Synthetic primer
augacgugggccccgagug

SEQ ID NO: 241 - RNA, 19 nt, Artificial Sequence
  Synthetic primer
cacucggggcccacgucau

SEQ ID NO: 242 - RNA, 19 nt, Artificial Sequence
  Synthetic primer
gacgugggccccgagugca

SEQ ID NO: 243 - RNA, 19 nt, Artificial Sequence
  Synthetic primer
ugcacucggggcccacguc

SEQ ID NO: 244 - RNA, 19 nt, Artificial Sequence
  Synthetic primer
acgugggccccgagugcaa

SEQ ID NO: 245 - RNA, 19 nt, Artificial Sequence
  Synthetic primer
uugcacucggggcccacgu

SEQ ID NO: 246 - RNA, 19 nt, Artificial Sequence
  Synthetic primer
cgugggccccgagugcaau

SEQ ID NO: 247 - RNA, 19 nt, Artificial Sequence
  Synthetic primer
auugcacucggggcccacg

SEQ ID NO: 248 - RNA, 19 nt, Artificial Sequence
  Synthetic primer
ugggccccgagugcaaucg

SEQ ID NO: 249 - RNA, 19 nt, Artificial Sequence
  Synthetic primer
cgauugcacucggggccca

SEQ ID NO: 250 - RNA, 19 nt, Artificial Sequence
  Synthetic primer
ggccccgagugcaaucgcg

SEQ ID NO: 251 - RNA, 19 nt, Artificial Sequence
  Synthetic primer
cgcgauugcacucggggcc

SEQ ID NO: 252 - RNA, 19 nt, Artificial Sequence
  Synthetic primer
gccccgagugcaaucgcgg

SEQ ID NO: 253 - RNA, 19 nt, Artificial Sequence
  Synthetic primer
ccgcgauugcacucggggc

SEQ ID NO: 254 - DNA, 360 bp, Artificial Sequence
  Synthetic primer
ttcaggaggatctttcagaagatgcgtttccaattttgagggggcgtcagctcttcaccggagacccaaatacaacaaatcaagtcgcctgccctggcgacactttcgaaggactggagtgggaatcagagcttcacgggttaaaaagccgatgtcacatcggccgttcgaaactcctcctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggct

SEQ ID NO: 255 - DNA, 23 bp, Artificial Sequence
  Synthetic primer
uuccagugcucugauggaadtdt

SEQ ID NO: 256 - DNA, 23 bp, Artificial Sequence
  Synthetic primer
uuccaucagagcacuggaadtdt

SEQ ID NO: 257 - DNA, 23 bp, Artificial Sequence
  Synthetic primer
aaaaucaccccacugcaaadtdt

SEQ ID NO: 258 - DNA, 23 bp, Artificial Sequence
  Synthetic primer
uuugcaguggggugauuuudtdt

SEQ ID NO: 259 - DNA, 23 bp, Artificial Sequence
  Synthetic primer
ccccacugcaaacuccuccdtdt

SEQ ID NO: 260 - DNA, 23 bp, Artificial Sequence
  Synthetic primer
ggaggaguuugcaguggggdtdt

SEQ ID NO: 261 - DNA, 23 bp, Artificial Sequence
  Synthetic primer
cacugcaaacuccucccccdtdt

SEQ ID NO: 262 - DNA, 23 bp, Artificial Sequence
  Synthetic primer
gggggaggaguuugcagugdtdt

SEQ ID NO: 263 - DNA, 23 bp, Artificial Sequence
  Synthetic primer
aaacuccucccccugcuagdtdt

SEQ ID NO: 264 - DNA, 23 bp, Artificial Sequence
  Synthetic primer
cuagcagggggaggaguuudtdt

SEQ ID NO: 265 - DNA, 23 bp, Artificial Sequence
  Synthetic primer
cuccucccccugcuagaaadtdt

SEQ ID NO: 266 - DNA, 23 bp, Artificial Sequence
  Synthetic primer
uuucuagcagggggaggagdtdt

SEQ ID NO: 267 - DNA, 23 bp, Artificial Sequence
  Synthetic primer
ucacauugaaaugcuguaadtdt

SEQ ID NO: 268 - DNA, 23 bp, Artificial Sequence
  Synthetic primer
uuacagcauuucaaugugadtdt

SEQ ID NO: 269 - DNA, 23 bp, Artificial Sequence
  Synthetic primer
gaaaugcuguaaaugacgudtdt

SEQ ID NO: 270 - DNA, 23 bp, Artificial Sequence
  Synthetic primer
acgucauuuacagcauuucdtdt

SEQ ID NO: 271 - DNA, 23 bp, Artificial Sequence
  Synthetic primer
ugcuguaaaugacgugggcdtdt

SEQ ID NO: 272 - DNA, 23 bp, Artificial Sequence
  Synthetic primer
gcccacgucauuuacagcadtdt

SEQ ID NO: 273 - DNA, 23 bp, Artificial Sequence
  Synthetic primer
uguaaaugacgugggccccdtdt

SEQ ID NO: 274 - DNA, 23 bp, Artificial Sequence
  Synthetic primer
ggggcccacgucauuuacadtdt

SEQ ID NO: 275 - DNA, 23 bp, Artificial Sequence
  Synthetic primer
aaaugacgugggccccgagdtdt

SEQ ID NO: 276 - DNA, 23 bp, Artificial Sequence
  Synthetic primer
cucggggcccacgucauuudtdt

SEQ ID NO: 277 - DNA, 23 bp, Artificial Sequence
  Synthetic primer
ugacgugggccccgagugcdtdt

SEQ ID NO: 278 - DNA, 23 bp, Artificial Sequence
  Synthetic primer
gcacucggggcccacgucadtdt

SEQ ID NO: 279 - DNA, 23 bp, Artificial Sequence
  Synthetic primer
gugggccccgagugcaaucdtdt

SEQ ID NO: 280 - DNA, 23 bp, Artificial Sequence
  Synthetic primer
gauugcacucggggcccacdtdt

SEQ ID NO: 281 - DNA, 23 bp, Artificial Sequence
  Synthetic primer
gggccccgagugcaaucgcdtdt

SEQ ID NO: 282 - DNA, 23 bp, Artificial Sequence
  Synthetic primer
gcgauugcacucggggcccdtdt

SEQ ID NO: 283 - DNA, 23 bp, Artificial Sequence
  Synthetic primer
ccccgagugcaaucgcgggdtdt

SEQ ID NO: 284 - DNA, 23 bp, Artificial Sequence
  Synthetic primer
cccgcgauugcacucggggdtdt

SEQ ID NO: 285 - DNA, 23 bp, Artificial Sequence
  Synthetic primer
ugcuuuaacuggcguuggcdtdt

SEQ ID NO: 286 - DNA, 23 bp, Artificial Sequence
  Synthetic primer
gccaacgccaguuaaagcadtdt

SEQ ID NO: 287 - DNA, 23 bp, Artificial Sequence
  Synthetic primer
gaacugcgguaacugaagudtdt

SEQ ID NO: 288 - DNA, 23 bp, Artificial Sequence
  Synthetic primer
acuucaguuaccgcaguucdtdt

SEQ ID NO: 289 - DNA, 23 bp, Artificial Sequence
  Synthetic primer
gagauuacgauugcugggcdtdt

SEQ ID NO: 290 - DNA, 23 bp, Artificial Sequence
  Synthetic primer
gcccagcaaucguaaucucdtdt

SEQ ID NO: 291 - DNA, 23 bp, Artificial Sequence
  Synthetic primer
gaaucgcuuagauuaagagdtdt

SEQ ID NO: 292 - DNA, 23 bp, Artificial Sequence
  Synthetic primer
cucuuaaucuaagcgauucdtdt

SEQ ID NO: 293 - DNA, 23 bp, Artificial Sequence
  Synthetic primer
ucgucaguggagucagagudtdt

SEQ ID NO: 294 - DNA, 23 bp, Artificial Sequence
  Synthetic primer
acucugacuccacugacgadtdt

SEQ ID NO: 295 - DNA, 23 bp, Artificial Sequence
  Synthetic primer
guggaucucacgguguagadtdt

SEQ ID NO: 296 - DNA, 23 bp, Artificial Sequence
  Synthetic primer
ucuacaccgugagauccacdtdt

SEQ ID NO: 297 - DNA, 23 bp, Artificial Sequence
  Synthetic primer
uagcuagcuaguagauaagdtdt

SEQ ID NO: 298 - DNA, 23 bp, Artificial Sequence
  Synthetic primer
cuuaucuacuagcuagcuadtdt

SEQ ID NO: 299 - DNA, 23 bp, Artificial Sequence
  Synthetic primer
ugcuguaaaugacgugggcdtdt

SEQ ID NO: 300 - DNA, 23 bp, Artificial Sequence
  Synthetic primer
gcccacgucauuuacagcadtdt

SEQ ID NO: 301 - DNA, 23 bp, Artificial Sequence
  Synthetic primer
ugcuguaaaugacgugggcdtdt

SEQ ID NO: 302 - DNA, 23 bp, Artificial Sequence
  Synthetic primer
gcccacgucauuuacagcadtdt

SEQ ID NO: 303 - DNA, 23 bp, Artificial Sequence
  Synthetic primer
gaaaugcuguaaaugacgudtdt

SEQ ID NO: 304 - DNA, 23 bp, Artificial Sequence
  Synthetic primer
acgucauuuacagcauuucdtdt

SEQ ID NO: 305 - DNA, 23 bp, Artificial Sequence
  Synthetic primer
gaaaugcuguaaaugacgudtdt

SEQ ID NO: 306 - DNA, 23 bp, Artificial Sequence
  Synthetic primer
acgucauuuacagcauuucdtdt

SEQ ID NO: 307 - DNA, 20 bp, Artificial Sequence
  Synthetic primer
tacaagtgggtctgcgatgg

SEQ ID NO: 308 - DNA, 20 bp, Artificial Sequence
  Synthetic primer
tgaagtccccggatttgcag

SEQ ID NO: 309 - DNA, 22 bp, Artificial Sequence
  Synthetic primer
ggacccctttgcttagatgaaa

SEQ ID NO: 310 - DNA, 21 bp, Artificial Sequence
  Synthetic primer
ccaccaagacctattgctctg

SEQ ID NO: 311 - DNA, 19 bp, Artificial Sequence
  Synthetic primer
agacccacctctcgcagtc

SEQ ID NO: 312 - DNA, 21 bp, Artificial Sequence
  Synthetic primer
ggagtcctcctcgatgtagtc

SEQ ID NO: 313 - DNA, 20 bp, Artificial Sequence
  Synthetic primer
aacggtcattcacccaggtc

SEQ ID NO: 314 - DNA, 21 bp, Artificial Sequence
  Synthetic primer
ggctgaagaataggagttgcc

SEQ ID NO: 315 - RNA, 21 nt, Artificial Sequence
  Synthetic primer
gucaucacacugaauaccaau

SEQ ID NO: 316 - RNA, 23 nt, Artificial Sequence
  Synthetic primer
cacaguagugugacuuaugguua

SEQ ID NO: 317 - RNA, 25 nt, Artificial Sequence
  Synthetic primer
cuacacaaaucagcgauuuccaugu

SEQ ID NO: 318 - RNA, 25 nt, Artificial Sequence
  Synthetic primer
gauguguuuagucgcuaaagguaca

SEQ ID NO: 319 - DNA, 95 bp, Artificial Sequence
  Synthetic primer
ttgaaaatcaccccactgcaaactcctccccctgctagaaacctcacattgaaatgctgtaaatgacgtgggccccgagtgcaatcgcgggaagc

SEQ ID NO: 320 - DNA, 21 bp, Artificial Sequence
  Synthetic primer
ugcuguaaaugacgugggctt

SEQ ID NO: 321 - DNA, 21 bp, Artificial Sequence
  Synthetic primer
ttacgacauuuacugcacccg

SEQ ID NO: 322 - DNA, 21 bp, Artificial Sequence
  Synthetic primer
gaaaugcuguaaaugacgutt

SEQ ID NO: 323 - DNA, 21 bp, Artificial Sequence
  Synthetic primer
ttcuuuacgacauuuacugca

SEQ ID NO: 324 - DNA, 21 bp, Artificial Sequence
  Synthetic primer
gagauuacgauugcugggctt

SEQ ID NO: 325 - DNA, 21 bp, Artificial Sequence
  Synthetic primer
ttcucuaaugcuaacgacccg

SEQ ID NO: 326 - DNA, 21 bp, Artificial Sequence
  Synthetic primer
ugcuuuaacuggcguuggctt

SEQ ID NO: 327 - DNA, 21 bp, Artificial Sequence
  Synthetic primer
ttacgaaauugaccgcaaccg

SEQ ID NO: 328 - DNA, 21 bp, Artificial Sequence
  Synthetic primer
gaacugcgguaacugaagutt

SEQ ID NO: 329 - DNA, 21 bp, Artificial Sequence
  Synthetic primer
ttcuugacgccauugacuuca

SEQ ID NO: 330 - DNA, 21 bp, Artificial Sequence
  Synthetic primer
gaaucgcuuagauuaagagtt

SEQ ID NO: 331 - DNA, 21 bp, Artificial Sequence
  Synthetic primer
ttcuuagcgaaucuaauucuc

SEQ ID NO: 332 - DNA, 21 bp, Artificial Sequence
  Synthetic primer
uccagaaaaugacgugggctt

SEQ ID NO: 333 - DNA, 21 bp, Artificial Sequence
  Synthetic primer
ttaggucuuuuacugcacccg

SEQ ID NO: 334 - DNA, 21 bp, Artificial Sequence
  Synthetic primer
gauaagguguaaaugacgutt

SEQ ID NO: 335 - DNA, 21 bp, Artificial Sequence
  Synthetic primer
ttcuauuccacauuuacugca

SEQ ID NO: 336 - DNA, 21 bp, Artificial Sequence
  Synthetic primer
ucgucaguggagucagagutt

SEQ ID NO: 337 - DNA, 21 bp, Artificial Sequence
  Synthetic primer
ttagcagucaccucagucuca

SEQ ID NO: 338 - DNA, 21 bp, Artificial Sequence
  Synthetic primer
ugcuguaaaugaggagcgctt

SEQ ID NO: 339 - DNA, 21 bp, Artificial Sequence
  Synthetic primer
ttacgacauuuacuccucgcg

SEQ ID NO: 340 - DNA, 21 bp, Artificial Sequence
  Synthetic primer
gaaaugcuguaauucaccutt

SEQ ID NO: 341 - DNA, 21 bp, Artificial Sequence
  Synthetic primer
ttcuuuacgacauuaagugga

SEQ ID NO: 342 - DNA, 21 bp, Artificial Sequence
  Synthetic primer
guggaucucacgguguagatt

SEQ ID NO: 343 - DNA, 21 bp, Artificial Sequence
  Synthetic primer
ttcaccuagagugccacaucu

SEQ ID NO: 344 - DNA, 21 bp, Artificial Sequence
  Synthetic primer
uagcuagcuaguagauaagtt

SEQ ID NO: 345 - DNA, 21 bp, Artificial Sequence
  Synthetic primer
ttaucgaucgaucaucuauuc

SEQ ID NO: 346 - DNA, 228 bp, Artificial Sequence
  Synthetic primer
ctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgcctt

SEQ ID NO: 347 - DNA, 211 bp, Artificial Sequence
  Synthetic primer
tccttctgggggcgagggcgactggagacccggatgtccagcctggaggtcaccgcgggctcaggggtcccgatccgctttgcgcgaccccagggcgccactgccatcctgagttgggtgcagtcccgggattccgccgcgtgctccgggacgggggccaccccctcccgcccctgcccccgcccctttggcccgccccccgaattccatt

SEQ ID NO: 348 - DNA, 201 bp, Artificial Sequence
  Synthetic primer
aatcaatatttacgtccagactccaggtatccgtacaattgatttttcagatgtttatactcagccaaaggcgggatcccacaaaacaaaaaatatttttttggctgtacttttgtgaagattttatttaaattcctgattgatcagtgtctattaggtgatttggaataacaatgtaaaaacaatatacaacgaaaggaa

SEQ ID NO: 349 - DNA, 1362 bp, Artificial Sequence
  Synthetic primer
cctgattgatcagtgtctattaggtgatttggaataacaatgtaaaaacaatatacaacgaaaggaagctaaaaatctatacacaattcctagaaaggaaaaggcaaatatagaaagtggcggaagttcccaacatttttagtgttttccttttgaggcagagaggacaatggcattaggctattggaggatcttgaaaggctgttgttatccttctgtggacaacaacagcaaaatgttaacagttaaacatcgagaaatttcaggaggatctttcagaagatgcgtttccaattttgagggggcgtcagctcttcaccggagacccaaatacaacaaatcaagtcgcctgccctggcgacactttcgaaggactggagtgggaatcagagcttcacgggttaaaaagccgatgtcacatcggccgttcgaaactcctcctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcaggtaaggcttgctccaggcgccagaataggttgagagggagcccccggggggcccttgggaatttatttttttgggtacaaataatcactccatccctgggagacttgtggggtaatggcacggggtccttcccaaacggctggagggggcgctggaggggggcgctgaggggagcgcgagggtcgggaggagtctgagggatttaagggaaacggggcaccgctgtcccccaagtctccacagggtgagggaccgcatcttctttgagacggagtctagctctgtcgcccaggatggagtgcagtggcacgatctcagctcactgcaacctccgcctcccgggtttaagcgagtctcctctctcagcctcccgaatagctgggattacaggcgcccaaccaccacgcccgcctaatttttgtatttttagtagagacgggttttcaccattttggccaggctggtctcgaaccccgacctcaggtgatctgcccaaaagtgctgggattacaggcgtcagccaccgcgcccggccgggaccctctcttctaactcggagctgggtgtggggacctccagtcctaaaacaagggatcactcccacccccgccttaagtccttctgggggcgagggcgactggagacccggatgtccagcctggaggtca

SEQ ID NO: 350 - DNA, 25 bp, Artificial Sequence
  Synthetic primer
actggaactcgtttctttcgcatct

SEQ ID NO: 351 - DNA, 23 bp, Artificial Sequence
  Synthetic primer
ccatcgcagacccacttgtagga

SEQ ID NO: 352 - DNA, 23 bp, Artificial Sequence
  Synthetic primer
tcgaaggactggagtgggaatca

SEQ ID NO: 353 - DNA, 27 bp, Artificial Sequence
  Synthetic primer
tgctagaaacctcacattgaaatgctg

SEQ ID NO: 354 - DNA, 23 bp, Artificial Sequence
  Synthetic primer
ccagggtttccagctaggacaca

SEQ ID NO: 355 - DNA, 28 bp, Artificial Sequence
  Synthetic primer
tcatttacagcatttcaatgtgaggttt

SEQ ID NO: 356 - DNA, 24 bp, Artificial Sequence
  Synthetic primer
ggggcccacgtcatttacagcatt

SEQ ID NO: 357 - DNA, 22 bp, Artificial Sequence
  Synthetic primer
agctggaaaccctggcttcccg

SEQ ID NO: 358 - DNA, 20 bp, Artificial Sequence
  Synthetic primer
tacaagtgggtctgcgatgg

SEQ ID NO: 359 - DNA, 20 bp, Artificial Sequence
  Synthetic primer
tgaagtccccggatttgcag

SEQ ID NO: 360 - DNA, 20 bp, Artificial Sequence
  Synthetic primer
gtcagctcttcaccggagac

SEQ ID NO: 361 - DNA, 21 bp, Artificial Sequence
  Synthetic primer
cactccagtccttcgaaagtg

SEQ ID NO: 362 - DNA, 21 bp, Artificial Sequence
  Synthetic primer
tttgaaaatcaccccactgca

SEQ ID NO: 363 - DNA, 30 bp, Artificial Sequence
  Synthetic primer
cctgattgatcagtgtctattaggtgattt

SEQ ID NO: 364 - DNA, 22 bp, Artificial Sequence
  Synthetic primer
tgacctccaggctggacatccg

SEQ ID NO: 365 - DNA, 26 bp, Artificial Sequence
  Synthetic primer
cagactccaggtatccgtacaattga

SEQ ID NO: 366 - DNA, 25 bp, Artificial Sequence
  Synthetic primer
gtggcctgttggactacacccaatg

SEQ ID NO: 367 - DNA, 21 bp, Artificial Sequence
  Synthetic primer
cctgctagaaacctcacattg

SEQ ID NO: 368 - DNA, 20 bp, Artificial Sequence
  Synthetic primer
ggatcacgacctgctgtgtc

SEQ ID NO: 369 - DNA, 23 bp, Artificial Sequence
  Synthetic primer
uccagaaaaugacgugggcdtdt

SEQ ID NO: 370 - DNA, 23 bp, Artificial Sequence
  Synthetic primer
gcccacgucauuuucuggadtdt

SEQ ID NO: 371 - DNA, 23 bp, Artificial Sequence
  Synthetic primer
ugcuguaaaugaggagcgcdtdt

SEQ ID NO: 372 - DNA, 23 bp, Artificial Sequence
  Synthetic primer
gcgcuccucauuuacagcadtdt

SEQ ID NO: 373 - DNA, 23 bp, Artificial Sequence
  Synthetic primer
gauaagguguaaaugacgudtdt

SEQ ID NO: 374 - DNA, 23 bp, Artificial Sequence
  Synthetic primer
acgucauuuacaccuuaucdtdt

SEQ ID NO: 375 - DNA, 23 bp, Artificial Sequence
  Synthetic primer
gaaaugcuguaauucaccudtdt

SEQ ID NO: 376 - DNA, 23 bp, Artificial Sequence
  Synthetic primer
aggugaauuacagcauuucdtdt

SEQ ID NO: 377 - RNA, 1442 nt, Artificial Sequence
  Synthetic primer
ggcaguggcgcccuggggucgcgcaaagcggaucgggaccccugagcccgcggugaccuccaggcuggacauccgggucuccagucgcccucgcccccagaaggacuuaaggcgggggugggagugaucccuuguuuuaggacuggagguccccacacccagcuccgaguuagaagagagggucccggccgggcgcgguggcugacgccuguaaucccagcacuuuugggcagaucaccugaggucgggguucgagaccagccuggccaaaauggugaaaacccgucucuacuaaaaauacaaaaauuaggcgggcguggugguugggcgccuguaaucccagcuauucgggaggcugagagaggagacucgcuuaaacccgggaggcggagguugcagugagcugagaucgugccacugcacuccauccugggcgacagagcuagacuccgucucaaagaagaugcggucccucacccuguggagacuugggggacagcggugccccguuucccuuaaaucccucagacuccucccgacccucgcgcuccccucagcgccccccuccagcgcccccuccagccguuugggaaggaccccgugccauuaccccacaagucucccagggauggagugauuauuuguacccaaaaaaauaaauucccaagggccccccgggggcucccucucaaccuauucuggcgccuggagcaagccuuaccugcaguccccgccgcggcgaggagcaaggcgacgguccagcgcaauuuccagccccagggccccaugcucgcagccucugccaggcagugucccgacccggaucacgaccugcuguguccuagcuggaaacccuggcuucccgcgauugcacucggggcccacgucauuuacagcauuucaaugugagguuucuagcagggggaggaguuugcaguggggugauuuucaaaugucuucaccucacugcaagaggaggaguuucgaacggccgaugugacaucggcuuuuuaacccgugaagcucugauucccacuccaguccuucgaaagugucgccagggcaggcgacuugauuuguuguauuugggucuccggugaagagcugacgcccccucaaaauuggaaacgcaucuucugaaagauccuccugaaauuucucgauguuuaacuguuaacauuuugcuguuguuguccacagaaggauaacaacagccuuucaagauccuccaauagccuaaugccauuguccucucugccucaaaaggaaaacacuaaaaauguugggaacuuccgccacuuucuauauuugccuuuuccuuucuaggaauuguguauagauuuuuagcuuccuuucguuguauauuguuuuuacauuguuauuccaaaucaccuaauagacacugaucaaucaggaauuuaaauaaaaucuucacaaaagua

SEQ ID NO: 378 - RNA, 1486 nt, Artificial Sequence
  Synthetic primer
ccggagcacgcggcggaaucccgggacugcacccaacucaggauggcaguggcgcccuggggucgcgcaaagcggaucgggaccccugagcccgcggugaccuccaggcuggacauccgggucuccagucgcccucgcccccagaaggacuuaaggcgggggugggagugaucccuuguuuuaggacuggagguccccacacccagcuccgaguuagaagagagggucccggccgggcgcgguggcugacgccuguaaucccagcacuuuugggcagaucaccugaggucgggguucgagaccagccuggccaaaauggugaaaacccgucucuacuaaaaauacaaaaauuaggcgggcguggugguugggcgccuguaaucccagcuauucgggaggcugagagaggagacucgcuuaaacccgggaggcggagguugcagugagcugagaucgugccacugcacuccauccugggcgacagagcuagacuccgucucaaagaagaugcggucccucacccuguggagacuugggggacagcggugccccguuucccuuaaaucccucagacuccucccgacccucgcgcuccccucagcgccccccuccagcgcccccuccagccguuugggaaggaccccgugccauuaccccacaagucucccagggauggagugauuauuuguacccaaaaaaauaaauucccaagggccccccgggggcucccucucaaccuauucuggcgccuggagcaagccuuaccugcaguccccgccgcggcgaggagcaaggcgacgguccagcgcaauuuccagccccagggccccaugcucgcagccucugccaggcagugucccgacccggaucacgaccugcuguguccuagcuggaaacccuggcuucccgcgauugcacucggggcccacgucauuuacagcauuucaaugugagguuucuagcagggggaggaguuugcaguggggugauuuucaaaugucuucaccucacugcaagaggaggaguuucgaacggccgaugugacaucggcuuuuuaacccgugaagcucugauucccacuccaguccuucgaaagugucgccagggcaggcgacuugauuuguuguauuugggucuccggugaagagcugacgcccccucaaaauuggaaacgcaucuucugaaagauccuccugaaauuucucgauguuuaacuguuaacauuuugcuguuguuguccacagaaggauaacaacagccuuucaagauccuccaauagccuaaugccauuguccucucugccucaaaaggaaaacacuaaaaauguugggaacuuccgccacuuucuauauuugccuuuuccuuucuaggaauuguguauagauuuuuagcuuccuuucguuguauauuguuuuuacauuguuauuccaaaucaccuaauagacacugaucaaucaggaauuuaaauaaaaucuucacaaaagua

(19) United States
(12) Patent Application Publication
(10) Pub. No.: US 2011/0110860 A1
(43) Pub. Date: May 12, 2011

(54) MODULATION OF LDL RECEPTOR GENE EXPRESSION WITH DOUBLE-STRANDED RNAS TARGETING THE LDL RECEPTOR GENE PROMOTER

(75) Inventors: DAVID R. COREY, Dallas, TX (US); Masayuki Matsui, Irving, TX (US); Muthiah Manoharan, Weston, MA (US); Sayda Elbashir, Cambridge, MA (US)

(73) Assignees: The Board of Regents of the University of Texas System, Austin, TX (US); Alnylam Pharmaceuticals Inc., Cambridge, MA (US)

(21) Appl. No.: 12/938,253

(22) Filed: Nov. 2, 2010

Related U.S. Application Data
(60) Provisional application No. 61/257,335, filed on Nov. 2, 2009.

(51) Int. Cl. A61K 31/713; C12N 5/00; C12Q 1/02; A61K 49/00; C12Q 1/68; G01N 33/53; A61P 9/00; A61P 9/10

(52) U.S. Cl. 424/9.1; 435/375; 514/44 R; 435/29; 435/6; 435/7.92

(57) ABSTRACT

Gene expression can be selectively regulated by double-stranded “antigene” RNAs that target regions of the low density lipoprotein receptor (LDL-R) promoter, thereby permitting modulation of LDL levels in vivo and subsequent effects on circulating LDL levels.

This application claims benefit of priority to U.S. Provisional Application Ser. No. 61/257,335, filed Nov. 2, 2009, the entire contents of which are hereby incorporated by reference.

### STATEMENT REGARDING FEDERALLY SPONSORED RESEARCH

This work was made with government support under grant GM077253 from the National Institutes of Health. The government has certain rights in this invention.

### I. FIELD OF THE INVENTION

The invention relates to modulating of gene expression using double-stranded oligonucleotides complementary to promoter regions of the low density lipoprotein receptor gene.

### II. BACKGROUND OF THE INVENTION

Synthetic small duplex RNAs complementary to gene promoters within chromosomal DNA are potent inhibitors or activators of target gene expression in mammalian cells (Morris et al., 2004; Ting et al., 2005; Janowski et al., 2005; Li et al., 2006; Janowski et al., 2007). These synthetic RNAs are called antigene RNAs (agRNAs) to distinguish them from small duplex RNAs that target mRNA. agRNAs recruit members of the argonaute (AGO) protein family to RNA transcripts that originate from the target gene promoter (Janowski et al., 2006; Kim et al., 2006; Han et al., 2007; Schwartz et al., 2008). Recognition of the target RNA occurs in close proximity to the chromosome, resulting in transcriptional, and possibly translational, modulation of the target gene's expression.

One remarkable feature of the synthetic agRNAs that the inventors have examined is the potency and robustness of their activity when they are introduced into cells. This potency, coupled with the presence of protein machinery that facilitates their function, suggests that endogenous small RNAs may possess the ability to recognize sequences within gene promoters. If an agRNA could direct proteins to specific gene promoters, such RNA-mediated modulation of transcription might have evolutionary advantages relative to the development of gene-specific protein transcription factors.

### SUMMARY OF THE INVENTION

Thus, in accordance with the present invention, there is provided a method of modulating expression of low density lipoprotein receptor (LDL-R) in a cell comprising contacting said cell with a first double-stranded RNA complementary to a portion of an LDL-R promoter. In one embodiment, the target gene is the LDL-R gene of a mammal (for example, a rodent, a primate, and the like). In some embodiments, the target gene is the LDL-R gene of a rat, a mouse, or a human. The first double-stranded RNA may increase LDL-R expression or decrease LDL-R expression. The direction of modulation will be related to the basal expression of LDL-R in the contacted cell. The double-stranded RNA may target a Repeat 2 region, a Repeat 3 region or both. The double-stranded RNA may target a sterol-independent regulatory element.

The portion may lie between −200 and −1 relative to a transcriptional start site of the LDL-R gene, between −100 and −1 relative to a transcriptional start site of the of the LDL-R gene, between −99 and −51, or between −50 and −1 relative to a transcriptional start site of the LDL-R gene, between −35 and −1 relative to a transcriptional start site of the LDL-R gene, or a region within bases +12 to −80, relative to the transcription start site of the LDL-R gene. In other embodiments, the portion may lie between positions x and y relative to the transcription start site of the target gene, wherein x can be anywhere between −200 and −15 relative to the transcription start site of the target gene, for example, −80, −79, −78, −77, −76, −74, −73, −72, −71, −70, −69, −67, −66, −64, −63, −62, −61, −60, −58, −57, −55, −54, −53, −52, −51, −50, −35, −34, −33, −32, −31, −30, −29, −27, −26, −25, −23, −22, −20, −19, −17 and −16 relative to the transcription start site of the target gene; and y can be anywhere between −61 and +12 relative to the transcription start site of the target gene, for example, −61, −60, −59, −58, −57, −55, −54, −53, −52, −51, −50, −48, −47, −45, −44, −43, −42, −41, −39, −38, −36, −35, −34, −33, −32, −31, −16, −15, −14, −13, −12, −11, −10, −8, −7, −6, −4, −3, −1, +1, +2, +3, +5, +6, +7, +9, +11 and +12 relative to the transcription start site of the target gene.

Alternatively, the double-stranded RNA may be defined in reference to an RNA transcript that is antisense to the strand encoding the promoter. This transcript may be defined as having a start site between +874 to +918 relative to the +1 transcription start site for LDLR mRNA. This transcript may also be defined as having 3′ end is the position at −568, or alternatively at positions −625 or −565. In particular, this transcript lies between positions +874 and −568 or positions +918 and −568, such as in SEQ ID NOS: 377 and 378 respectively, or between positions +918 and −625, as in SEQ ID NO: 379.

The double-stranded RNA may contain one or more modified nucleosides, such as a 2′-OMe nucleoside or a 2′-F nucleoside. One strand of the double-stranded RNA may contain one or more modified nucleosides, and the other strand may not contain a modified nucleoside. One strand of the double-stranded RNA may contain one or more 2′-OMe nucleoside, and the other strand may contain a 2′-F nucleoside. Alternatively, both strands of the double-stranded RNA may contain one or more 2′-OMe nucleosides, or both strands of the double-stranded RNA may contain one or more 2′-F nucleosides. The agRNA may further comprise one or more deoxyribonucleotides. The agRNA may comprise an overhang. The overhang may be a dinucleotide overhang, such as, for example, a dTdT dinucleotide overhang of each strand. The double-stranded RNA may comprise at least one phosphorothioate linkage in each strand. The double-stranded RNA may be 18-23 nucleotides in length. The double-stranded RNA may be formulated in a lipid vehicle.

The cell may be located in situ in a host, and the contacting step may be effected by administering to the host an effective amount of the double-stranded RNA. The method may further comprise detecting a change in the expression of LDL-R, such as by inferring a change in the expression from a physiologic change in the cell. The cell may be located in situ in a host and detecting may comprise inferring a change in the expression from a physiologic change in the host. Detecting may comprise one or more of Northern blot, PCR, immunohistochemistry, Western blot or ELISA.

The method may further comprise contacting said cell with a second agent that increases LDL expression. The second agent may be a second double-stranded RNA complementary to a portion of an LDL-R promoter that is distinct from the first double-stranded

RNA complementary to a portion of an LDL-R promoter. The method may also further comprise repeating the contacting of said cell with the first a double-stranded RNA.

In another embodiment, there is provided a method of reducing circulating low density lipoprotein in a subject comprising administering to said subject a first double-stranded RNA complementary to a portion of an LDL-R promoter. The subject may suffer from hypercholesterolemia, from atherosclerosis, and/or from coronary heart disease. The method may further comprise administering to said subject a second agent that reduces circulating low density lipoprotein. The second agent may be a double-stranded RNA complementary to a portion of an LDL-R promoter that is distinct from said first double-stranded RNA, or the second agent may be a statin or niacin. The statin may be lovastatin, atorvastatin, cerivastatin, fluvastatin, mevastatin, pitavastatin, pravastatin, rosuvastatin and simvastatin. Administering may comprise repeated administrations of the first double-stranded RNA. The double-stranded RNA may be formulated in a lipid vehicle.

In yet another embodiment, there is provided a pharmaceutical formulation comprising (a) a double-stranded RNA complementary to a portion of a low density lipoprotein receptor (LDL-R) promoter, and (b) a pharmaceutically acceptable buffer, carrier or diluent. In some embodiments, the pharmaceutical formulation comprises (a) a double-stranded RNA of 18 to 23 nucleotides complementary to a region of the low density lipoprotein receptor promoter located −1 to −200 relative to the transcriptional start site, said double-stranded RNA comprising one or more modified bases, and (b) a pharmaceutically acceptable buffer, carrier or diluent. In certain aspects, the region may be located within bases −1 to −100, relative to the transcription start site, of the LDL-R promoter, within bases +12 to −80, relative to the transcription start site, or within bases −1 to −35, relative to the transcription start site, of the LDL-R promoter. The one or more modified nucleosides may be 2′-OMe and/or 2′-F nucleosides. The double-stranded RNA may be complementary to at least a portion of a sterol-independent regulatory element.

In various embodiments, the expression of the target gene may be increased or decreased. The method may further comprise detecting a change in the expression of the target gene, for example, by inferring a change in the expression of the target gene from a physiologic change in the cell, or by detecting comprises one or more of Northern blot, PCR, immunohistochemistry, Western blot or ELISA. The cell may be located in situ in a host and the detecting may comprise inferring a change in the expression of the target gene from a physiologic change in the host.

The invention provides compositions such as reagents and formulations tailored to the subject methods. It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These and other embodiments of the invention will be better understood when considered in conjunction with the following description and the accompanying drawings. It should be understood that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-E—Transcripts at the LDLR promoter. (FIG. 1A) Location of gene specific primers used in RACE. (FIG. 1B) Analysis of RACE products defining the 5′ termini of LDLR mRNA. Primer+10836 and primer+10793 are gene specific primers complementary to exon 2 in LDLR mRNA. Positive control is a product (˜900-bp) from a RACE using HeLa RT template and a control primer specific for β-actin cDNA. (FIG. 1C) Analysis of 5′ and 3′ RACE products for sense or antisense noncoding transcripts. Nested PCRs were performed to increase specificity in amplification of target cDNAs. Gene specific primers used in the 1st/2nd(nested) PCRs are shown on top of each lane. (FIG. 1D) Relative locations of LDLR mRNA and the antisense transcript. (FIG. 1E) Relative expression levels of LDLR mRNA and the antisense transcript evaluated by qRT-PCR. ***, P<0.001 (unpaired t-test). Error shown is SD. The transcription start sites and the 3′ ends identified by these RACE analyses are shown in FIGS. 8A-C. Results showing the connection between the 5′ and 3′ RACE products are presented in FIG. 8D. The sequence of the antisense transcript is shown in FIG. 8E.

FIGS. 2A-G—Identification and characterization of agRNAs that activate LDLR expression. (FIG. 2A) Location of target sites for agRNAs relative to the +1 transcription start site for LDLR (SEQ ID NO:319). (FIG. 2B) Western analysis showing the effects of varied agRNAs (50 nM) on expression of LDLR in HepG2 cells. (FIG. 2C) Quantitation of results shown in panel B and independent replicates (n=4). Statistical significance relative to mismatch control LDLRmm1 was tested by paired t-test. *, P<0.05; **, P<0.01; ***, P<0.001 (FIG. 2D) Western blots showing a dose response for LDLR-24(U/U). (FIG. 2E) Western blots showing a time course of LDLR expression after treatment with LDLR-24(U/U) (50 nM). (FIG. 2F) ChIP of RNAP II using an anti-RNAP II antibody after treatment with activating agRNAs or mismatch control (50 nM). n=3. Data were analyzed using Dunnett's test. **, P<0.01 relative to mismatch control LDLRmm1. (FIG. 2G) RIP of AGO1 or AGO2 using an anti-AGO1 or anti-AGO2 antibody after treatment with activating agRNAs or mismatch control (50 nM). Error shown is SD. See also FIGS. 9 and 12.

FIGS. 3A-B—Effect of mismatch-containing duplexes on expression of LDLR. (FIG. 3A) The sequences of LDLR-24(U/U), LDLR-28(U/U), and corresponding mismatch oligomers. The upper strands are sense strands and the lower strands are antisense strands. Mismatch bases for LDLRmm1-6 are represented by red, bold face letters. Scrambled oligomers were generated by randomly scrambling the sequence of LDLR-24 or LDLR-28 (SEQ ID NOS:320-345). (FIG. 3B) Western analyses of LDLR expression for LDLR-24(U/U), LDLR-28(U/U), mismatch-containing oligomers LDLRmm1-6, and Scr1-5 (50 nM). NT indicates no treatment. Western blots are representative from at least three independent replicates for each experiment. See also FIG. 10.

FIGS. 4A-E—Effect of chemical modifications on RNA-mediated activation of LDLR. (FIG. 4A) Structures of 2′-O-methyl RNA and 2′-fluoro RNA. (FIG. 4B) Effect of 2′-O-methyl and 2′-fluoro modifications on activation by LDLR-24 (50 nM). Representative western blots (top) and quantification of three independent replicates (bottom) are shown. (FIG. 4C) Effect of 2′-O-methyl and 2′-fluoro modifications on activation by LDLR-28 (50 nM). Representative western blots (top) and quantification of three independent replicates (bottom) are shown. (FIG. 4D) Western blots showing a dose response for LDLR-24(U/O). (FIG. 4E) Western blots showing a time course profile of LDLR expression after treatment with LDLR-24(U/O) (50 nM) in HepG2 cells. Statistical significance relative to mismatch control LDLRmm1 was evaluated by paired t-test. *, P<0.05; **, P<0.01. Error shown is SD. See also FIGS. 11 and 12.

FIGS. 5A-C—Binding of LDL to cell surface LDLR. (FIG. 5A) Fluorescent microscopy of HepG2 cells four days after transfection of LDLR-24(U/U) or LDLRmm1 (50 nM), or no treatment. Cells were treated with DiI-LDL (12 μg/mL) or a mixture of DiI-LDL (12 μg/mL) and unlabeled LDL (120 μg/mL) at 4° C. for 2 h. (FIG. 5B) Flow cytometry showing DiI-LDL association. Varying concentrations of LDLR-24(U/U), LDLR-28(U/U), or LDLRmm1 were transfected into HepG2 cells. Four days after transfection, cells were treated with DiI-LDL (12 μg/mL) at 4° C. for 2 h and fluorescence from DiI-LDL bound to cells was measured by FACScan. (FIG. 5C) Quantitation of cell surface-bound DiI-LDL after treatments shown in (B). Mean fluorescence value for no treatment sample was expressed as 100%. Error shown is SEM. n=5.

FIGS. 6A-D—Effect of treatment with activating agRNAs or poly I:C on expression of interferon responsive genes and LDLR. (FIG. 6A) Western analysis showing effect of activating agRNAs (50 nM) or poly I:C (100 ng/mL) on LDLR expression. (FIG. 6B) qRT-PCR analysis showing effect of activating agRNAs or poly I:C on the expression of interferon responsive genes using cells examined in panel A. n=3. (FIG. 6C) Western blots showing effect of poly I:C on LDLR expression. (FIG. 6D) qRT-PCR analysis showing effect of poly I:C on the expression of interferon responsive genes using cells examined in FIG. 6C. n=3. Western blots are representative from three independent replicates. Error shown is SD.

FIGS. 7A-B—Combination treatment of activating agRNAs and 25-hydroxycholesterol or lovastatin. 50 nM duplex RNAs were used in these experiments. (FIG. 7A) 25-Hydroxycholesterol (2 μM) or EtOH (vehicle) was added to cell culture media two days after transfection of activating agRNA LDLR-24(U/U), LDLR-28(U/U), or a mismatch oligomer LDLRmm1. Data shown are western blots of LDLR expression on Day 4 (left) and quantitation of five independent replicates (right). Statistical significance was evaluated by paired t-test. *, P<0.05; ***, P<0.001 relative to mismatch control LDLRmm1. (FIG. 7B) Lovastatin (10 or 30 μM) or EtOH (vehicle) was added to cell culture media two days after transfection of activating agRNAs or a mismatch oligomer. Data shown are western blots of LDLR expression on Day 4 (left) and quantitation of three independent replicates (right). Upregulation of LDLR expression by LDLR-24(U/U) or lovastatin was statistically significant (two-way ANOVA; P<0.01). No significant interaction effects were detected between the two different treatments using agRNAs and lovastatin. NT indicates no treatment. Error shown is SD. See also FIG. 13.

FIGS. 8A-E—Data from RACE analyses. (FIG. 8A) Transcription start sites for LDLR mRNA in HepG2 cells. 5′ RACE PCR products (˜200-bp; FIG. 1B) for LDLR mRNA were excised from the gel and subjected to cloning and sequencing (SEQ ID NO:346). (FIG. 8B) Transcription start sites of the antisense transcript. 5′ RACE PCR products (˜900-bp; FIG. 1C) for the antisense transcript were excised from the gel and subjected to cloning and sequencing (SEQ ID NO:347). (FIG. 8C) 3′ ends of the antisense transcript. 3′ RACE PCR product (˜600-bp; FIG. 1C) for the antisense transcript was excised from the gel and subjected to cloning and sequencing (SEQ ID NO:348). (FIG. 8D) Amplification of the antisense transcript to check the connection between the 5′ and 3′ RACE PCR products for the antisense transcript. Total RNAs from HepG2 cells were treated with DNase I prior to reverse transcriptions. The RNAs (2 μg) were reverse-transcribed using an oligo dT primer and Superscript III reverse transcriptase to generate cDNAs (+RT). No reverse transcription was performed for —RT negative control. PCRs (50 μL) were conducted using different combinations of primers targeting the inside or outside of the antisense transcript. The reaction mixture (50 μL) contained cDNA(100 ng) or genomic DNA (50 ng), forward/reverse primers (A, B, C, or D; 0.2 μM), dNTPs (0.2 mM), 10× high fidelity PCR buffer, MgSO₄ (2 mM), and Platinum Taq DNA polymerase high fidelity (2.5 U). The thermal cycling profile includes an initial denaturation step at 94° C. for 2 min, followed by 45 cycles of 94° C. for 30 sec, 66° C. for 30 sec, and 68° C. for 2 min. The PCR products were analyzed on 1% agarose gel. (FIG. 8E) The sequence of the PCR product (˜1400-bp) amplified using Primer A+B and +RT template (SEQ ID NO:349).

FIGS. 9A-E—Supplemental data for unmodified agRNAs. (FIG. 9A) Western blots showing a dose response profile for LDLR-28(U/U). (FIG. 9B) Western blots showing a time course profile of LDLR expression after treatment with LDLR-28(U/U) (50 nM) in HepG2 cells. (FIG. 9C) qPCR analysis for the antisense transcript three days after transfection of agRNAs or mismatch control (50 nM). Two primer sets, Primer-235/−160 and Primer-79/+53, were used in qPCR. n=4. (FIG. 9D) ChIP for H3K27 trimethylation (H3K27me3) marker within the LDLR gene locus. n=4. (FIG. 9E) Activation of LDLR in other cell lines. agRNAs LDLR-24(U/U), LDLR-28(U/U), LDLR+807, and LDLRmm1 (50 nM) were transfected into HuH-7 (hepatocellular carcinoma cells), GM04281 (fibroblast cells), and SW480 (colorectal cancer cells). Cells were harvested on Day 3 (HuH-7) or Day 4 (GM04281, SW480) for western analysis. Data shown are representative from at least three independent experiments. Statistical significance was evaluated by unpaired t-test. **, P<0.01; ***, P<0.001 relative to mismatch control LDLRmm1. NT indicates no treatment. Error shown is SD.

FIGS. 10A-J—Dose response profiles, time course profiles, and RIP for mismatch-containing oligomers. (FIGS. 10A-G) Western blots showing dose response profiles for LDLRmm1 (FIG. 10A), LDLRmm2 (FIG. 10B), Scr1 (FIG. 10C), Scr2 (FIG. 10D), Scr3 (FIG. 10E), Scr4 (FIG. 10F), and Scr5 (FIG. 10G). (FIGS. 10H-I) Western blots showing time course profiles of LDLR expression after treatment with LDLRmm1 (FIG. 10H) or LDLRmm2 (FIG. 10I) in HepG2 cells. Western blots shown are representative from at least three independent replicates. (FIG. 10J) RIP of AGO1 or AGO2 using an anti-AGO1 or anti-AGO2 antibody after treatment with an activating agRNA (LDLR-24(U/U)) or mismatch controls (LDLRmm1, LDLRmm3, and LDLRmm4) (50 nM). RIP data shown are representative from three independent experiments.

FIGS. 11A-E—Supplemental data for chemically modified agRNAs. (FIGS. 11A-C) Western blots showing dose response profiles for LDLR-24(F/U), LDLR-28(U/O), and LDLR-28(F/U). (FIG. 11D) qPCR analysis for the antisense transcript three days after transfection of agRNAs or mismatch control (50 nM). n=4. (FIG. 11E) ChIP for RNAP II after treatment with chemically modified agRNAs or mismatch control (50 nM). n=3. Data shown are representative from at least three independent experiments. Statistical significance was evaluated by paired t-test. *, P<0.05; **, P<0.01 relative to mismatch control LDLRmm1. Error shown is SD.

FIG. 12—Half-maximal effective concentration (EC₅₀) and maximal fold activation (Aₘₐₓ) for activating agRNAs. Dose response data were fit to the following model equation: y=1+(a-1)*x/(b+x), where y is -fold activation and x is concentration of duplex RNA. a and b are fitting parameters, where a and b are taken as the Aₘₐₓ and EC₅₀ values, respectively. Error shown is SEM. EC₅₀+/−SEM and Aₘₐₓ+/−SEM obtained from curve fittings are shown on top of each graph.

FIG. 13—Combination treatment of activating chemically modified agRNAs and 25-hydroxycholesterol. 25-Hydroxycholesterol (2 μM) or EtOH (vehicle) was added to cell culture media two days after transfection of activating agRNA LDLR-24(U/O), LDLR-28(F/U), or mismatch control LDLRmm1 (50 nM). Data shown are western blots of LDLR expression on Day 4 (left) and quantitation of five independent replicates (right). Statistical significance was evaluated by paired t-test. **, P<0.01; ***, P<0.001 relative to mismatch control LDLRmm1. NT indicates no treatment. Error shown is SD.

FIG. 14—Bioinformatics to select promoter siRNAs. Design criteria for mouse LDL-R duplexes are summarized. The promoter region of the mouse LDL-R gene (SEQ ID NO:2) was compared with the corresponding sequences from the rat (SEQ ID NO:4) and human genes (SEQ ID NO:3); duplexes were designed starting from −99 relative to the transcription start site (TSS) at every third position; for regions in which sequence homology existed with human and/or rat sequences, a duplex was designed at every position (SEQ ID NOS:5-13).

FIG. 15—List of sense/antisense sequences for mouse agRNAs. Shown are the duplex name, start position, and the sense and antisense sequences for the mouse LDL-R duplexes used; also indicated are whether homology exists and the region of homology with corresponding human and rat LDL-R promoter sequences. Each sequence further comprises a dTdT at the 3′ end of both strands (SEQ ID NOS:14-101).

FIGS. 16A-D—In vitro single dose screening of ag-RNA-mLDR. Duplexes targeting the mouse LDL-R promoter region were screened in vitro in BNL-Cl.2 cells (FIG. 16A), Hepa 1c1c7 cells (FIG. 16B), Hepa 1-6 cells (FIG. 16C), and N-Muli cells (FIG. 16D). Data indicate the LDL-R mRNA levels relative to controls.

FIG. 17—In vitro single dose screening of ag-RNA-mLDR. The effect of mouse ag-RNAs shown in FIG. 15 on LDL-R mRNA levels were tested in vitro in four cell lines; the numbers show the LDL-R transcript levels relative to controls, as well as the standard deviation. An average of three experiments is shown.

FIGS. 18A-B—hLDL-R activation in HepG2 cells. The effect of various duplexes targeting the hLDL-R promoter on the LDL-R mRNA levels is shown. FIG. 18A shows the effect of target location; FIG. 18B shows the effect of strand modification.

FIGS. 19A-B—hLDL-R activation in Hep3B cells. The effect of various duplexes targeting the hLDL-R promoter on the LDL-R mRNA levels is shown. FIG. 19A shows the effect of target location; FIG. 19B shows the effect of strand modification.

FIGS. 20A-B—hLDL-R activation in HepG2 cells. The levels of LDL-R mRNA (FIG. 20A) and protein (FIG. 20B) relative to controls in cells treated with various LDL-R ag-RNA are shown.

FIG. 21—hLDL-R activation in HepG2 and Hep3B cells. Summarized are effect of modified and unmodified duplexes targeting different regions of the hLDL-R promoter on the LDL-R mRNA levels relative to control.

FIG. 22—Cytokine response of unmodified and modified duplexes. Two out of three unmodified agRNAs induce INF-α but not modified agRNAs.

FIG. 23—Cytokine response of unmodified and modified duplexes. Two out of three unmodified agRNAs induce TNF-α but not modified agRNAs.

FIG. 24—Cytokine response of unmodified and modified duplexes. Two out of three unmodified agRNAs induce IL-1β but not modified agRNAs.

FIG. 25—Cytokine response of unmodified and modified duplexes. Two out of three unmodified agRNAs induce IL-6 but not modified agRNAs.

FIG. 26—agRNAs/agRNA for LDL-R-activation study. Both strands modified with 2′-fluoro or 2′-O-methyl at pyrimidines (SEQ ID NOS:102-131).

FIG. 27—agRNA for LDL-R-activation (SEQ ID NOS:132-157).

FIG. 28—Additional agRNAs that target the human LDL-R (SEQ ID NOS:158-253).

FIG. 29—Sequence of a portion of the human LDL-R promoter sequence. The transcription start site (+1) is indicated in bold underline (SEQ ID NO:254).

### DETAILED DESCRIPTION OF SPECIFIC EMBODIMENTS OF THE INVENTION

The members of the low density lipoprotein (LDL) receptor gene family bind a broad spectrum of extracellular ligands. Traditionally, they had been regarded as mere cargo receptors that promote the endocytosis and lysosomal delivery of these ligands. However, recent genetic experiments have revealed critical functions for LDL receptor family members in the transmission of extracellular signals and the activation of intracellular tyrosine kinases. This process regulates neuronal migration and is crucial for brain development. Signaling through these receptors has been reported to require the interaction of their cytoplasmic tails with a number of intracellular adaptor proteins, including Disabled-1 (Dab1) and FE65. Nonetheless, a key role for LDL receptors remains the regulation of circulating lipoprotein levels. Upregulation of LDL-R can decrease plasma LDL-c and thus is effective at treating hypercholesterolemia, and major contributor to atherosclerosis and heart disease.

The ability of small synthetic or endogenous RNAs to inhibit gene expression by targeting mRNA is well established (Siomi and Siomi, 2009). Recently several reports have appeared suggesting that small RNA that are complementary to gene promoters can also regulate gene expression. These antigene RNAs (agRNAs) (terminology to distinguish RNAs complementary to mRNA) can either inhibit or activate gene expression depending on the sequence being targeted and the basal expression level of the target gene.

Gene silencing by double-stranded RNAs complementary to mRNA has rapidly moved from the laboratory to the clinical. Gene silencing can also be achieved by a related technology, single-stranded antisense oligonucleotides, and the advantages of duplex RNAs will need to be addressed on a case by case basis. Single-stranded antisense oligonucleotides that target mRNA can be used to enhance expression of chosen isoforms by blocking splice sites, but cannot yield increased expression of the target protein. Gene activation by RNA would, therefore, expand the pool of genes whose expression might be manipulated for experimental and therapeutic benefit.

The inventors chose to examine RNA-mediated gene activation of a therapeutically significant gene, LDL-R. The basis for this choice was four-fold: 1) experimental or clinical data showing that enhanced expression of the target gene leads to a potentially favorable therapeutic outcome; 2) expressed in the liver, an organ demonstrated to be accessible using current technology for in vivo RNA delivery; 3) expressed at detectable levels; and 4) the ability to modulate the target gene expression by changing cellular environment should be well established. Such perturbations can be used to study the effects of agRNAs and provide reassurance that enhanced expression is possible.

Taking these criteria into consideration the inventors chose the LDL receptor (LDL-R) as a target for agRNAs. LDL-R is a cell surface receptor responsible for internalization of plasma LDL-cholesterol (LDL-c). Enhanced expression of hepatic LDL-R decreases the level of plasma LDL-c, providing a route for treatment of hypercholesterolemia. LDL-R expression can be detected in a variety of liver cell lines and can be modulated by different treatment. LDL-R expression is repressed by addition of 25-hydroxycholeserol, and is enhanced by addition of lovastatin, an inhibitor of HMG CoA reductase. In this study, they observed activation of LDL-R expression by duplex RNAs.

Thus, the present invention provides a general method of selectively modulating (increasing or decreasing) expression (i.e., transcription) of an LDL-R gene use of agRNAs targeting the LDR-R promoter region. In a particular embodiment, the LDL-R gene may be located in a cell in situ in a host, and the contacting step may be effected by administering to the host an effective amount of the agRNA.

Various aspects of the invention, as set forth above, are described in greater detail in the following paragraphs.

### I. agRNAs and Production Thereof

A. agRNAs

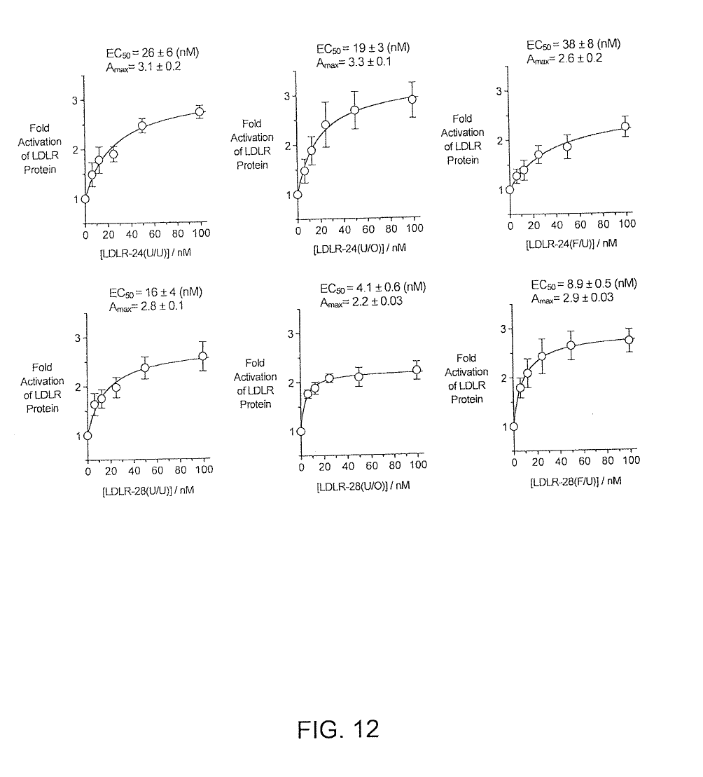

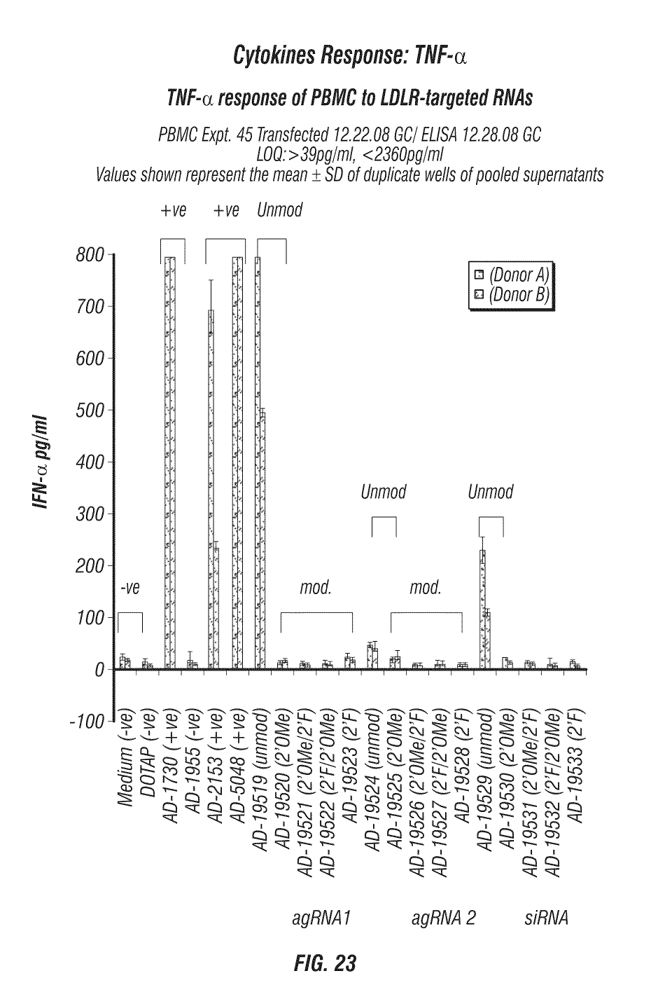

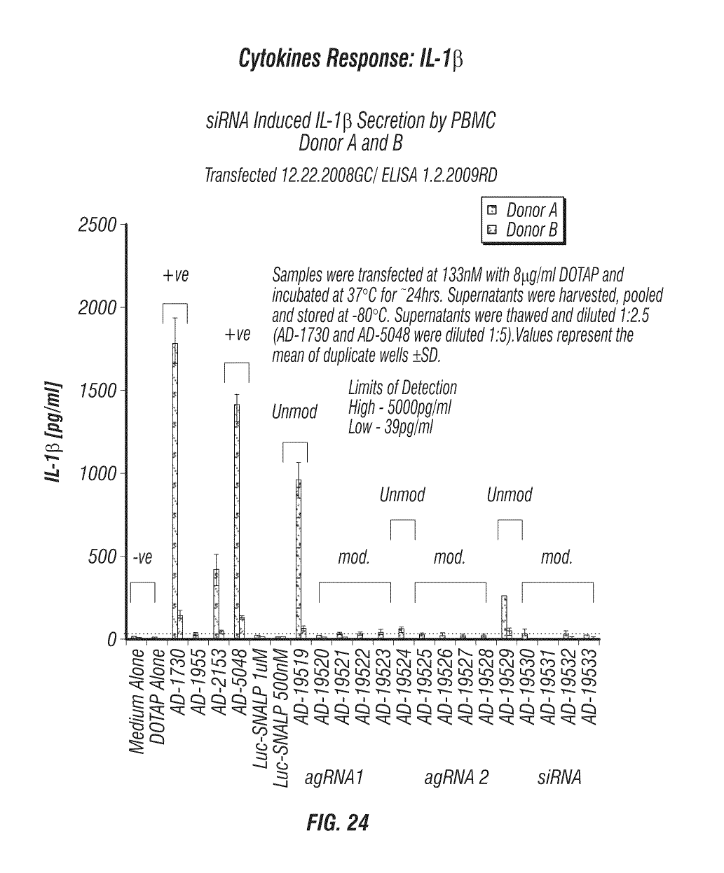

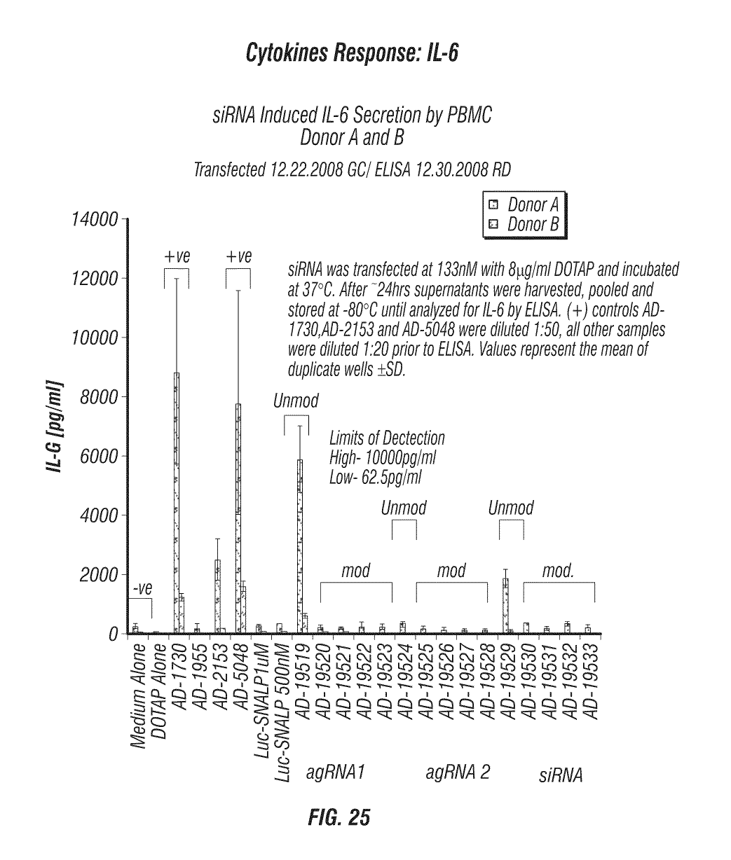

In general, agRNAs are defined as double-stranded, partially double-stranded and hairpin structured oligonucleotides. In particular, an agRNA will includes a nucleotide sequence sufficiently complementary to hybridize to 12-23 nucleotides from a promoter target sequence. Exemplary LDL-R-targeting sequences are provided in Table 1, FIG. 12, and FIGS. 23-25. The double-stranded RNAs of the present invention are segments of 12-30 bases in length that are designed to target the LDL-R promoter in target cells. In particular, ranges of 12-23, 15-30, 15-23, 18-23, 19-23, 20-23 and 21-23 bases are contemplated, as are specific lengths of 12, 13, 14, 15, 16, 17, 18, 19, 20, 21, 22, 23, 24 and 25 bases.

Oligonucleotides are chemically synthesized using nucleoside phosphoramidites. A phosphoramidite is a derivative of natural or synthetic nucleoside with protection groups added to its reactive exocyclic amine and hydroxy groups. The naturally-occurring nucleotides (nucleoside-3′-phosphates) are insufficiently reactive to afford the synthetic preparation of oligonucleotides. A dramatically more reactive (2-cyanoethyl) N,N-diisopropyl phosphoramidite group is therefore attached to the 3′-hydroxy group of a nucleoside to form nucleoside phosphoramidite. The protection groups prevent unwanted side reactions or facilitate the formation of the desired product during synthesis. The 5′-hydroxyl group is protected by DMT (dimethoxytrityl) group, the phosphite group by a diisopropylamino (iPr2N) group and a 2-cyanoethyl (OCH₂CH₂CN) group. The nucleic bases also have protecting groups on the exocyclic amine groups (benzoyl, acetyl, isobutyryl, or many other groups). In RNA synthesis, the 2′ group is protected with a TBDMS (t-butyldimethylsilyl) group or with a TOM (t-butyldimethylsilyloxymethyl) group. With the completion of the synthesis process, all the protection groups are removed.

Whereas enzymes synthesize RNA in a 5′ to 3′ direction, chemical RNA synthesis is performed backwards in a 3′ to 5′ reaction. Based on the desired nucleotide sequence of the product, the phosphoramidites of nucleosides A, C, G, and T are added sequentially to react with the growing chain in a repeating cycle until the sequence is complete. In each cycle, the product's 5′-hydroxy group is deprotected and a new base is added for extension. In solid-phase synthesis, the oligonucleotide being assembled is bound, via its 3′-terminal hydroxy group, to a solid support material on which all reactions take place. The 3′ group of the first base is immobilized via a linker onto a solid support (most often, controlled pore glass particles or macroporouspolystyrene beads). This allows for easy addition and removal of reactants. In each cycle, several solutions containing reagents required for the elongation of the oligonucleotide chain by one nucleotide residue are sequentially pumped through the column from an attached reagent delivery system and removed by washing with an inert solvent.

agRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. In one embodiment, the agRNA includes a non-nucleotide moiety, e.g., a cholesterol moiety. The non-nucleotide moiety can be attached to the 3′ or 5′ end of the oligonucleotide agent.

A wide variety of well-known, alternative oligonucleotide chemistries may be used (see, e.g., U.S. Patent Publications 2007/0213292, 2008/0032945, 2007/0287831, etc.), particularly single-stranded complementary oligonucleotides comprising 2′-methoxyethyl, 2′-fluoro, and morpholino bases (see e.g., Summerton & Weller, 1997). The oligonucleotide may include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). Also contemplated are locked nucleic acid (LNA) and peptide nucleic acids (PNA).

A locked nucleic acid (LNA), often referred to as inaccessible RNA, is a modified RNA nucleotide (Elmén et al., 2008). The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. Such oligomers are commercially available. The locked ribose conformation enhances base stacking and backbone pre-organization. This significantly increases the thermal stability (melting temperature) of oligonucleotides (Kaur et al., 2006). LNA bases may be included in a DNA backbone, by they can also be in a backbone of LNA, 2′-O-methyl RNA, 2′-methoxyethyl RNA, or 2′-fluoro RNA. These molecules may utilize either a phosphodiester or phosphorothioate backbone.

Other oligonucleotide modifications can be made to produce oligonucleotides. For example, stability against nuclease degradation has been achieved by introducing a phosphorothioate (P═S) backbone linkage at the 3′ end for exonuclease resistance and 2′ modifications (2′-OMe, 2′-F and related) for endonuclease resistance (WO 2005115481; Li et al., 2005; Choung et al., 2006). A motif having entirely of 2′-O-methyl and 2′-fluoro nucleotides has shown enhanced plasma stability and increased in vitro potency (Allerson et al., 2005). The incorporation of 2′-O-Me and 2′-O-MOE does not have a notable effect on activity (Prakash et al., 2005).

Sequences containing a 4′-thioribose modification have been shown to have a stability 600 times greater than that of natural RNA (Hoshika et al, 2004). Crystal structure studies reveal that 4′-thioriboses adopt conformations very similar to the C3′-endo pucker observed for unmodified sugars in the native duplex (Haeberli et al., 2005). Stretches of 4′-thio-RNA were well tolerated in both the guide and nonguide strands. However, optimization of both the number and the placement of 4′-thioribonucleosides is necessary for maximal potency.

In the boranophosphate linkage, a non-bridging phosphodiester oxygen is replaced by an isoelectronic borane (BH3-) moiety. Boranophosphate siRNAs have been synthesized by enzymatic routes using T7 RNA polymerase and a boranophosphate ribonucleoside triphosphate in the transcription reaction. Boranophosphate siRNAs are more active than native siRNAs if the center of the guide strand is not modified, and they may be at least ten times more nuclease resistant than unmodified siRNAs (Hall et al., 2004; Hall et al., 2006).

Certain terminal conjugates have been reported to improve or direct cellular uptake. For example, NAAs conjugated with cholesterol improve in vitro and in vivo cell permeation in liver cells (Rand et al., 2005). Soutschek et al. (2004) have reported on the use of chemically-stabilized and cholesterol-conjugated siRNAs have markedly improved pharmacological properties in vitro and in vivo. Chemically-stabilized siRNAs with partial phosphorothioate backbone and 2′-O-methyl sugar modifications on the sense and antisense strands (discussed above) showed significantly enhanced resistance towards degradation by exo- and endonucleases in serum and in tissue homogenates, and the conjugation of cholesterol to the 3′ end of the sense strand of an oligonucleotides by means of a pyrrolidine linker does not result in a significant loss of gene-silencing activity in cell culture. These study demonstrates that cholesterol conjugation significantly improves in vivo pharmacological properties of oligonucleotides.

U.S. Patent Publication 2008/0015162, incorporated herein by reference, provide additional examples of nucleic acid analogs useful in the present invention. The following excerpts are derived from that document and are exemplary in nature only.

In certain embodiments, oligomeric compounds comprise one or more modified monomers, including 2′-modified sugars, such as BNA's and monomers (e.g., nucleosides and nucleotides) with 2′-substituents such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(˜O)—N(Rₘ)(Rₙ), where each Rₘ and Rₙ is, independently, H or substituted or unsubstituted C₁-C₁₀ alkyl.

Certain BNA's have been prepared and disclosed in the patent literature as well as in scientific literature (Singh et al., 1998; Koshkin et al., 1998; Wahlestedt et al., 2000; Kumar et al., 1998; WO 94/14226; WO 2005/021570; Singh et al, 1998; examples of issued US patents and published applications that disclose BNA s include, for example, U.S. Pat. Nos. 7,053,207; 6,268,490; 6,770,748; 6,794,499; 7,034,133; and 6,525,191; and U.S. Patent Publication Nos. 2004/0171570; 2004/0219565; 2004/0014959; 2003/0207841; 2004/0143114; and 2003/0082807.

Also provided herein are BNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., 2001; Braasch et al., 2001; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) BNA is used for the bicyclic moiety; in the case of an ethylene group in this position, the term ethyleneoxy (4′-CH₂CH₂—O-2′) BNA is used (Singh et al., 1998; Morita et al., 2003). Methyleneoxy (4′-CH₂—O-2′) BNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., 2000).

An isomer of methyleneoxy (4′-CH₂—O-2′) BNA that has also been discussed is α-L-methyleneoxy (4′-CH₂—O-2′) BNA which has been shown to have superior stability against a 3′-exonuclease. The α-L-methyleneoxy (4′-CH₂—O-2′) BNA's were incorporated into antisense gapmers and chimeras that showed potent antisense activity (Frieden et al., 2003).

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., 1998). BNAs and preparation thereof are also described in WO 98/39352 and WO 99/14226.

Analogs of methyleneoxy (4′-CH₂—O-2′) BNA, phosphorothioate-methyleneoxy (4′-CH₂—O-2′) BNA and 2′-thio-BNAs, have also been prepared (Kumar et al., 1998). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., 1998). In addition, 2′-amino- and 2′-methylamino-BNA's have been prepared and the thermal stability of their duplexes with complementary RNA and DNA strands has been previously reported.

Modified sugar moieties are well known and can be used to alter, typically increase, the affinity of oligomers for targets and/or increase nuclease resistance. A representative list of modified sugars includes, but is not limited to, bicyclic modified sugars (BNA's), including methyleneoxy (4′-CH₂—O-2′) BNA and ethyleneoxy (4′-(CH₂)₂—O-2′ bridge) BNA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

The naturally-occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleotide backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

Described herein are linking groups that link monomers (including, but not limited to, modified and unmodified nucleosides and nucleotides) together, thereby forming an oligomeric compound. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

### II. The LDL Receptor and Promoter

A. Hypercholesterolemia

Hypercholesterolemia is characterized by the presence of high levels of cholesterol in the blood. It is not a disease but a metabolic derangement that can be secondary to many diseases and can contribute to many forms of disease, most notably cardiovascular disease. It is closely related to the terms “hyperlipidemia” (elevated levels of lipids) and “hyperlipoproteinemia” (elevated levels of lipoproteins).

Elevated cholesterol in the blood is due to abnormalities in the levels of lipoproteins, the particles that carry cholesterol in the bloodstream. This may be related to diet, genetic factors (such as LDL receptor mutations in familial hypercholesterolemia) and the presence of other diseases such as diabetes and an underactive thyroid. The type of hypercholesterolemia depends on which type of particle (such as low density lipoprotein) is present in excess. High cholesterol levels are treated with diets low in cholesterol, medications, and rarely with other treatments including surgery (for particular severe subtypes). This is also increased emphasis on other risk factors for cardiovascular disease, such as high blood pressure.

Elevated cholesterol does not lead to specific symptoms unless it has been long-standing. Some types of hypercholesterolemia lead to specific physical findings: xanthoma (deposition of cholesterol in patches on the skin or in tendons), xanthelasma palpabrum (yellowish patches around the eyelids) and arcus senilis (white discoloration of the peripheral cornea). Long-standing elevated hypercholesterolemia leads to accelerated atherosclerosis; this can express itself in a number of cardiovascular diseases: coronary artery disease (angina pectoris, heart attacks), stroke and short stroke-like episodes and peripheral vascular disease.

There is no specific level at which cholesterol levels are abnormal. Cholesterol levels are found in a continuum within a population. Higher cholesterol levels lead to increased risk of specific disease, most notably cardiovascular diseases. Specifically, high LDL cholesterol levels are associated with increased risk. When speaking of hypercholesterolemia, most people are referring to high levels of LDL cholesterol.

When measuring cholesterol, it is important to measure its subfractions before drawing a conclusion as to the cause of the problem. The subfractions are LDL, HDL and VLDL. In the past, LDL and VLDL levels were rarely measured directly due to cost concerns. VLDL levels are reflected in the levels of triglycerides (generally about 45% of triglycerides is composed of VLDL). LDL was usually estimated as a calculated value from the other fractions (total cholesterol minus HDL and VLDL); this method is called the Friedewald calculation; to be specific: LDL˜=Total Cholesterol−HDL −(0.2× Triglycerides).

Less expensive (and less accurate) laboratory methods and the Friedewald calculation have long been used because of the complexity, labor, and expense of the electrophoretic methods developed in the 1970s to identify the different lipoprotein particles that transport cholesterol in the blood. With time, more advanced laboratory analyses that do measure LDL and VLDL particle sizes and levels have been developed, and at far lower cost. These have partly been developed and become more popular as a result of the increasing clinical trial evidence that intentionally changing cholesterol transport patterns, including to certain abnormal values compared to most adults, often has a dramatic effect on reducing, even partially reversing, the atherosclerotic process.

While part of the circulating cholesterol originates from diet, and restricting cholesterol intake may reduce blood cholesterol levels, there are various other links between the dietary pattern and cholesterol levels. The American Heart Association compiles a list of the acceptable and unacceptable foods for those who are diagnosed with hypercholesterolemia. Dietary changes can potentially be very strong. When a group of Tarahumara Indians from Mexico with no obesity or cholesterol problems were exposed to a Western diet, their risk profile worsened significantly, with cholesterol levels rising over thirty percent.

Evidence is accumulating that eating more carbohydrates—especially simpler, more refined carbohydrates—increases levels of triglycerides in the blood, lowers HDL, and may shift the LDL particle distribution pattern into unhealthy atherogenic patterns. An increasing number of researchers are suggesting that a major dietary risk factor for cardiovascular diseases is trans fatty acids, and in the US the FDA has revised food labeling requirements to include listing trans fat quantities.

Clinical evidence has summarized treatment for both primary prevention and secondary prevention. Two factors that have been put forward for consideration when choosing therapy are the patient's risk of coronary disease and their lipoprotein pattern.

Risk of coronary disease. To calculate the benefit of treatment, there are two online calculators that can estimate baseline risk. Combining the baseline risk with the relative risk reduction of a treatment can lead to the absolute risk reduction of number needed to treat. For example, one of the calculators projects that a patient had a 10% risk of coronary disease over ten years. As noted below, the relative risk reduction of a statin is 30%. Thus, after 4-7 years of treatment with a statin, a patient's risk will drop to 7%. This equates to an absolute risk reduction of 3%, or a number needed to treat of 33. Thirty three such patients must be treated for 4-7 years for one to benefit.

Lipoprotein patterns. The treatment depends on the type of hypercholesterolemia. Clinical trials, starting in the 1970s, have repeatedly and increasingly found that normal cholesterol values do not necessarily reflect healthy cholesterol values. This has increasingly lead to the newer concept of dyslipidemia, despite normo-cholesterolemia. Thus there has been increasing recognition of the importance of “lipoprotein subclass analysis” as an important approach to better understand and change the connection between cholesterol transport and atherosclerosis progression. Fredrickson Types IIa and IIb can be treated with diet, statins (most prominently rosuvastatin, atorvastatin, simvastatin, or pravastatin), cholesterol absorption inhibitors (ezetimibe), fibrates (gemfibrozil, bezafibrate, fenofibrate or ciprofibrate), vitamin B3 (niacin), bile acid sequestrants (colestipol, cholestyramine), LDL apheresis and in hereditary severe cases liver transplantation.

Treatments. In strictly controlled surroundings, such as a hospital ward dedicated to metabolism problems, a diet can reduce cholesterol levels by 15%. In practice, dietary advice can provide a modest decrease in cholesterol levels and may be sufficient in the treatment of mildly elevated cholesterol.

Many primary physicians and heart specialists will initially prescribe medication in combination with diet and exercise. According to various resources, statins are the most commonly used and effective forms of medication for the treatment of high cholesterol. The U.S. Preventive Services Task Force (USPSTF) estimated that after 5 to 7 years of treatment, the relative risk reduction by statins on coronary heart disease events is decreased by approximately 30%. More recently, a meta-analysis reported an almost identical relative risk reduction of 29.2% in low risk patients treated for 4.3 years. A relative risk reduction of 19% in coronary mortality was found in a meta-analysis of patients at all levels of risk.

B. LDL-Receptor

The Low-Density Lipoprotein (LDL) Receptor is a mosaic protein that mediates the endocytosis of cholesterol-rich LDL. It is a cell-surface receptor that recognizes the apoprotein B100 which is embedded in the phospholipid outer layer of LDL particles. The receptor also recognizes the apoE protein found in chylomicron remnants and VLDL remnants (IDL).

LDL receptor complexes are present in clathrin-coated pits (or buds) on the cell surface, which when bound to LDL-cholesterol via adaptin, are pinched off to form clathrin-coated vesicles inside the cell. This allows LDL-cholesterol to be bound and internalized in a process known as endocytosis and prevents the LDL just diffusing around the membrane surface. This occurs in all nucleated cells (not erythrocytes), but mainly in the liver which removes ˜70% of LDL from the circulation. LDL is directly involved in the development of atherosclerosis, due to accumulation of LDL-cholesterol in the blood. Atherosclerosis is the process responsible for the majority of cardiovascular diseases.

Once the coated vesicle is internalized it will shed its clathrin coat and will fuse with an acidic late endosome. The change in pH causes a conformational change in the receptor that releases the bound LDL particle. The receptors are then either destroyed or they can be recycled via the endocytic cycle back to the surface of the cell where the neutral pH will cause the receptor to revert to its native conformation ready to receive another LDL particle.

Synthesis of receptors in the cell is regulated by the level of free intracellular cholesterol; if it is in excess for the needs of the cell then the transcription of the receptor gene will be inhibited. LDL receptors are translated by ribosomes on the endoplasmic reticulum and are modified by the Golgi apparatus before travelling in vesicles to the cell surface.

The LDL receptor can be described as a chimeric protein. It is made up of a number of functionally distinct domains that can function independently of each other. The N-terminus of the LDL receptor contains a class A domain that is composed of seven sequence repeats (˜50% identical) each ˜40 amino acids long, with 6 cysteine residues. These ligand binding (LB) regions fold autonomously when synthesised as individual peptides. The cysteine residues form disulfide bonds forming an octahedral lattice, coordinated to a calcium ion, in each repeat. The exact mechanism of interaction between the LB repeats and ligand (LDL) is unknown, but it is thought that the repeats act as “grabbers” to hold the LDL. Binding of ApoB requires repeats 2-7 while binding ApoE requires only repeat 5 (thought to be the ancestral repeat).

Next to the ligand binding domain is an epidermal growth factor (EGF) precursor homology domain (EGFP domain). This shows approximately 30% homology with the EGF precursor gene. There are three “growth factor” repeats; A, B and C. A and B are closely linked while C is separated by a beta-propeller motif (LDL-R class B domain). The EGFP domain has been implicated in release of ligands bound to the receptor. It is thought that a conformational change occurs in the acidic (pH 5.0) conditions of the endosome bringing the beta-propeller into contact with ligand-binding repeats 4 and 5.

A third domain of the protein is rich in O-linked oligosaccharides but appears to show little function. Knockout experiments have confirmed that no significant loss of activity occurs without this domain. It has been speculated that the domain may have ancestrally acted as a spacer to push the receptor beyond the extracellular matrix.

A membrane spanning domain containing a chain of hydrophobic amino acid residues crosses the plasma membrane of the cell. Inside the cell the C-terminus domain contains a signal sequence that is needed for receptor internalization.

The gene coding the LDL receptor is split into 18 exons. Exon 1 contains a signal sequence that localises the receptor to the endoplasmic reticulum for transport to the cell surface. Beyond this, exons 2-6 code the ligand binding region; 7-14 code the EGFP domain; 15 codes the oligosaccharide rich region; 16 (and some of 17) code the membrane spanning region; and 18 (with the rest of 17) code the cytosolic domain.

C. Promoter

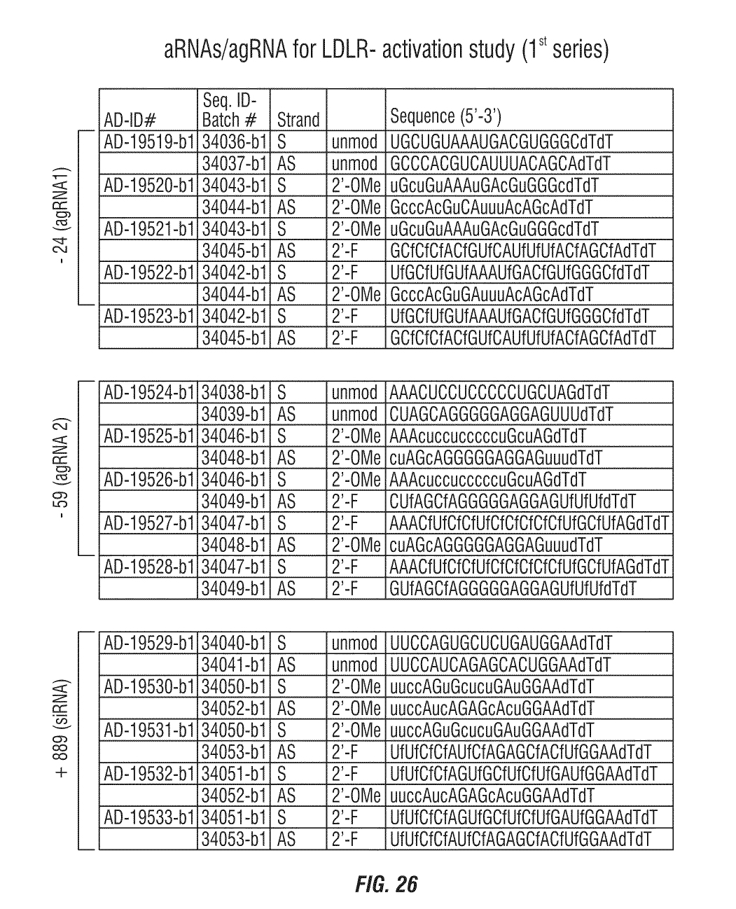

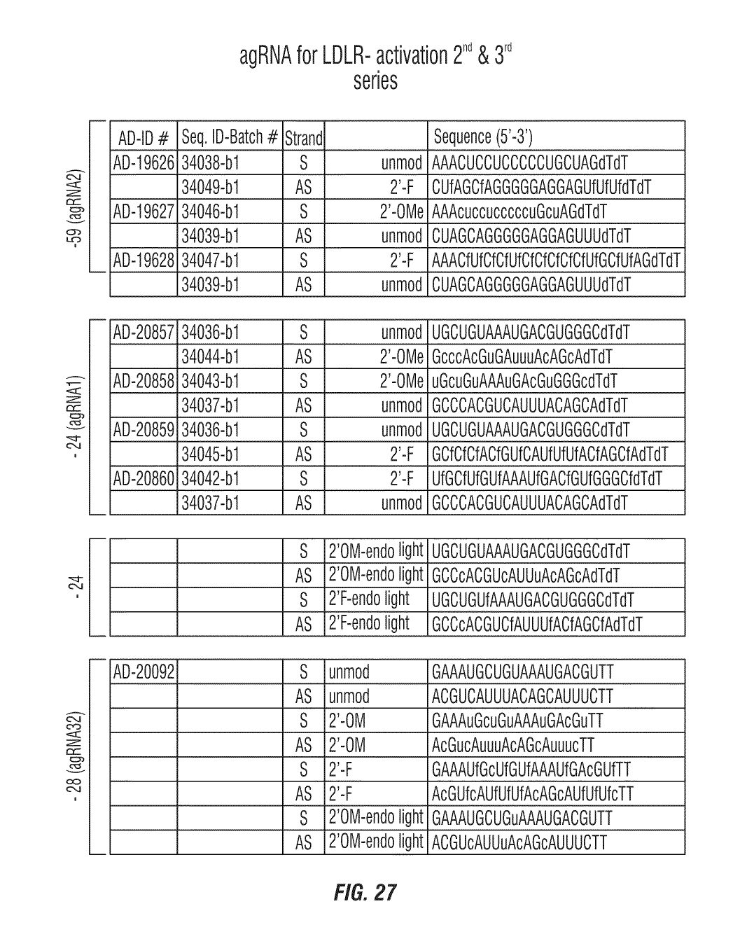

A portion of the human LDL-R promoter is shown in FIG. 26. It is characterized by three 16-base repeat regions, termed Regions 1, 2 and 3 that are between −109 and −44 relative to the transcriptional start site. It also contains a sterol-independent regulatory element downstream of the third repeat region, lying −30 to −8 relative to the transcriptional start site.

### III. Formulations and Delivery of Oligonucleotides

A. Cell Delivery

A variety of methods may be used to deliver oligonucleotides, including agRNAs, into a target cell. For cells in vitro embodiments, delivery can often be accomplished by direct injection into cells, and delivery can often be enhanced using hydrophobic or cationic carriers. Alternatively, the cells can be permeabilized with a permeabilization and then contacted with the oligonucleotide. The agRNA can be administered to the subject either as a naked oligonucleotide agent, in conjunction with a delivery reagent, or as a recombinant plasmid or viral vector which expresses the oligonucleotide agent.

For cells in situ, several applicable delivery methods are well-established, e.g., Elmen et al. (2008), Akinc et al. (2008); Esau et al. (2006), Krützfeldt et al. (2005). In particular, cationic lipids (see e.g., Hassani et al., 2004) and polymers such as polyethylenimine (see e.g., Urban-Klein, 2005) have been used to facilitate oligonucleotide delivery. Compositions consisting essentially of the oligomer (i.e., the oligomer in a carrier solution without any other active ingredients) can be directly injected into the host (see e.g., Tyler et al., 1999; McMahon et al., 2002). In vivo applications of duplex RNAs are reviewed in Paroo and Corey (2004).

When microinjection is not an option, delivery can be enhanced in some cases by using Lipofectamine™ (Invitrogen, Carlsbad, Calif.). PNA oligomers can be introduced into cells in vitro by complexing them with partially complementary DNA oligonucleotides and cationic lipid. The lipid promotes internalization of the DNA, while the PNA enters as cargo and is subsequently released. Peptides such as penetratin, transportan, Tat peptide, nuclear localization signal (NLS), and others, can be attached to the oligomer to promote cellular uptake (see e.g., Nielsen, 2004; Kaihatsu et al., 2003; Kaihatsu et al., 2004). Alternatively, the cells can be permeabilized with a permeabilization agent such as lysolecithin, and then contacted with the oligomer.

B. Routes of Administration

A composition that includes an agRNA can be delivered to a subject by a variety of routes. Exemplary routes include inhalation, parenchymal, subcutaneous, nasal, buccal and oral delivery. Also contemplated are delivery is through local administration directly to a disease site, or by systemic administration, e.g., parental administration. Parenteral administration includes intravenous (drip), subcutaneous, intraperitoneal or intramuscular injection, or intrathecal or intraventricular administration.

An agRNA featured in the invention can be administered to the subject by any means suitable for delivering the agent to the cells of the tissue at or near the area of target nucleic acid expression. Exemplary delivery methods include administration by gene gun, electroporation, or other suitable parenteral administration route.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection); subcutaneous injection or deposition including subcutaneous infusion (such as by osmotic pumps); direct application to the area at or near the site of disease, for example by a catheter or other placement device.

C. Formulations

An agRNA can be incorporated into pharmaceutical compositions suitable for administration. For example, compositions can include one or more oligonucleotide agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic. An agRNA featured in the invention may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be utilized versus a prolonged release composition. Alternatively, for certain preventative or long-term treatments, a sustained release composition may be appropriate. An agRNA can include a delivery vehicle, such as liposomes, for administration to a subject, carriers and diluents and their salts, and/or can be present in pharmaceutically acceptable formulations.

The agRNA agents featured by the invention may be formulated as pharmaceutical compositions prior to administering to a subject, according to techniques known in the art. Pharmaceutical compositions featured in the present invention are characterized as being at least sterile and pyrogen-free. As used herein, “pharmaceutical formulations” include formulations for human and veterinary use. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al., the entire disclosures of which are herein incorporated by reference.

The present pharmaceutical formulations include an agRNA featured in the invention (e.g., 0.1 to 90% by weight), or a physiologically acceptable salt thereof, mixed with a physiologically acceptable carrier medium. Particular physiologically acceptable carrier media are water, buffered water, normal saline, 0.4% saline, 0.3% glycine, hyaluronic acid and the like.

Pharmaceutical compositions featured in the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions can be packaged for use in liquid form, or can be lyophilized.

For solid compositions, conventional non-toxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, in particular 25%-75%, of one or more agents featured in the invention.

The invention also features the use of a composition that includes surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., 1995; Ishiwata et al., 1995).

The long-circulating liposomes enhance the pharmacokinetics and pharmacodynamics of DNA and RNA, particularly compared to conventional cationic liposomes which are known to accumulate in tissues of the MPS (Liu et al., 1995; PCT Publication No. WO 96/10391; PCT Publication No. WO 96/10390; PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also features compositions prepared for storage or administration that include a pharmaceutically effective amount of the desired oligonucleotides in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The nucleic acid molecules of the present invention can also be administered to a subject in combination with other therapeutic compounds to increase the overall therapeutic effect. The use of multiple compounds to treat an indication can increase the beneficial effects while reducing the presence of side effects. For example, use of statins in conjunction with the agRNAs of the present invention is contemplated.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. A particular group of carbohydrates includes lactose, threhalose, raffinose maltodextrins, and mannitol. Suitable polypeptides include aspartame. Amino acids include alanine and glycine, with glycine being specifically contemplated.

Suitable pH adjusters or buffers include organic salts prepared from organic acids and bases, such as sodium citrate, sodium ascorbate, and the like.

D. Dosage

An agRNA can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of agRNA (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of agRNA per kg of bodyweight. The unit dose, for example, can be administered by injection (e.g., intravenous or intramuscular, intrathecally, or directly into an organ), inhalation, or a topical application.

Delivery of an agRNA directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

Significant modulation of target gene expression may be achieved using nanomolar/submicromolar or picomolar/subnamomolar concentrations of the oligonucleotide, and it is typical to use the lowest concentration possible to achieve the desired resultant increased synthesis, e.g., oligonucleotide concentrations in the 1-100 nM range are contemplated; more particularly, the concentration is in the 1-50 nM, 1-25 nM, 1-10 nM, or picomolar range. In particular embodiments, the contacting step is implemented by contacting the cell with a composition consisting essentially of the oligonucleotide.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen.

An agRNA featured in the invention can be administered in a single dose or in multiple doses. Where the administration of the agRNA is by infusion, the infusion can be a single sustained dose or can be delivered by multiple infusions. Injection of the agent can be directly into the tissue at or near the site of aberrant or unwanted target gene expression (e.g., aberrant or unwanted miRNA or pre-miRNA expression). Multiple injections of the agent can be made into the tissue at or near the site.

In a particular dosage regimen, the agRNA is injected at or near a site of unwanted target nucleic acid expression once a day for seven days. Where a dosage regimen comprises multiple administrations, it is understood that the effective amount of agRNA administered to the subject can include the total amount of agRNA administered over the entire dosage regimen. One skilled in the art will appreciate that the exact individual dosages may be adjusted somewhat depending on a variety of factors, including the specific agRNA being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of an agRNA. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of the agRNA used for treatment may increase or decrease over the course of a particular treatment. Changes in dosage may result and become apparent from the results of diagnostic assays. For example, the subject can be monitored after administering an agRNA composition. Based on information from the monitoring, an additional amount of the agRNA composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀'s found to be effective in in vitro and in vivo animal models.

### IV. Detecting Expression

The detecting step is implemented by detecting a significant change in the expression of LDL-R, for example, by detecting at least a 10%, 25%, 50%, 200% or 500% increase in expression of LDL-R, or at least a 10%, 25%, 50%, 75%, or 90% decrease in expression of LDR-R, relative to a negative control, such as basal expression levels.

Detection may be effected by a variety of routine methods, such as directly measuring a change in the level of the target gene mRNA transcript, or indirectly detecting increased or decreased levels of the corresponding encoded protein compared to a negative control. Alternatively, resultant selective modulation of target gene expression may be inferred from phenotypic or physiologic changes that are indicative of increased or decreased expression of LDL-R.

A. Nucleic Acid Detection

Assessing expression may involve quantitating mRNA. Northern blotting techniques are well known to those of skill in the art. Northern blotting involves the use of RNA as a target. Briefly, a probe is used to target an RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished.

Nucleic acids may be quantitated following gel separation and staining with ethidium bromide and visualization under UV light. Alternatively, if the nucleic acid results from a synthesis or amplification using integral radio- or fluorometrically-labeled nucleotides, the products can then be exposed to x-ray film or visualized under the appropriate stimulating spectra, following separation.

In one embodiment, visualization is achieved indirectly. Following separation of nucleic acids, a labeled nucleic acid is brought into contact with the target sequence. The probe is conjugated to a chromophore or a radiolabel. In another embodiment, the probe is conjugated to a binding partner, such as an antibody or biotin, and the other member of the binding pair carries a detectable moiety.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present invention provides methods by which any or all of these types of analyses may be used. Using the sequences disclosed herein, oligonucleotide primers may be designed to permit the amplification of sequences throughout the Killin gene that may then be analyzed by direct sequencing.

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

B. Protein Detection

Immunodetection. Antibodies can be used in characterizing protein expression in cells through techniques such as ELISAs and Western blotting. For example, antibodies may be immobilized onto a selected surface, such as a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for the target that differs the first antibody. Appropriate conditions include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2-4 hrs, at temperatures on the order of about 25°-27° C. Following incubation, the antisera-contacted surface is washed so as to remove non-immunocomplexed material. A particular washing procedure includes washing with a solution such as PBS/Tween®, or borate buffer.

To provide a detecting means, the second antibody may have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will also find use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel- or fluorescently-tagged secondary antibodies against the toxin moiety are considered to be of particular use in this regard.

Mass Spectrometry. By exploiting the intrinsic properties of mass and charge, mass spectrometry (MS) can resolve and confidently identify a wide variety of complex compounds, including nucleic acids and proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000).

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to the tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances, sample clean up is required (Nelson et al., 1994; Gobom et al., 2000).

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode.

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff, 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Jiang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers.

### V. Examples

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Rapid Amplification of cDNA Ends (RACE) Analysis. RACE was performed using the GeneRacer Kit (Invitrogen). cDNA samples from HepG2 cells were prepared according to the kit manufacturer's protocol. The 5′ or 3′ end of cDNA was amplified through two nested PCR steps using Platinum Taq DNA Polymerase High Fidelity (Invitrogen) and appropriate primer sets (Table 51). The thermal cycling condition of the first PCR was: 94° C. for 2 min, followed by 5 cycles of 94° C. for 30 sec and 72° C. for 1 min, 5 cycles of 94° C. for 30 sec and 70° C. for 1 min, and 25 cycles of 94° C. for 30 sec, 66° C. for 30 sec, and 68° C. for 1 min. The condition of the following nested PCR was: 94° C. for 2 min, followed by 20 cycles of 94° C. for 30 sec, 65° C. for 30 sec, and 68° C. for 1 min. After gel purification, the PCR products were cloned into a pCR4-TOPO vector and sequenced (McDermott sequencing core, UT Southwestern).

TABLE 1
Sequences of primers used in RACE, qPCR and ChIP/RIP experiments

| Experiments | Name | Seq. | Sequence (5′ - - - 3′) |
|---|---|---|---|
| 5′ RACE (sense) | Primer+10793 | 350 | ACTGGAACTCGTTTCTTTCGCATCT |
| 5′ RACE (sense) | Primer+10836 | 351 | CCATCGCAGACCCACTTGTAGGA |
| 5′ RACE (antisense) | Primer-175 | 352 | TCGAAGGACTGGAGTGGGAATCA |
| 5′ RACE (antisense) | Primer-46 | 353 | TGCTAGAAACCTCACATTGAAATGCTG |
| 5′ RACE (antisense) | Primer+17 | 354 | CCAGGGTTTCCAGCTAGGACACA |
| 5′ RACE (sense)/ | Primer-31 | 355 | TCATTTACAGCATTTCAATGTGAGGTTT |
| 3′ RACE (antisense) | | | |
| 5′ RACE (sense)/ | Primer-3 | 356 | GGGGCCCACGTCATTTACAGCATT |
| 3′ RACE (antisense) | | | |
| 5′ RACE (sense)/ | Primer+31 | 357 | AGCTGGAAACCCTGGCTTCCCG |
| 3′ RACE (antisense) | | | |
| qPCR (LDLR mRNA) | LDLR exon2/3 | 358 | TACAAGTGGGTCTGCGATGG |
| | F | | |
| qPCR (LDLR mRNA) | LDLR exon2/3 | 359 | TGAAGTCCCCGGATTTGCAG |
| | R | | |
| qPCR (antisense | Primer-235 | 360 | GTCAGCTCTTCACCGGAGAC |
| transcript)/RIP | | | |
| qPCR (antisense | Primer-160 | 361 | CACTCCAGTCCTTCGAAAGTG |
| transcript)/RIP | | | |
| qPCR (antisense | Primer-79 | 362 | TTTGAAAATCACCCCACTGCA |
| transcript) | | | |
| Amplification of the | Primer A | 363 | CCTGATTGATCAGTGTCTATTAGGTGATTT |
| antisense transcript | (−541) | | |
| Amplification of the | Primer B | 364 | TGACCTCCAGGCTGGACATCCG |
| antisense transcript | (+821) | | |
| Amplification of the | Primer C | 365 | CAGACTCCAGGTATCCGTACAATTGA |
| antisense transcript | (−659) | | |
| Amplification of the | Primer D | 366 | GTGGCCTGTTGGACTACACCCAATG |
| antisense transcript | (+1001) | | |
| ChIP | Primer-48 | 367 | CCTGCTAGAAACCTCACATTG |
| ChIP/qPCR (antisense | Primer+53 | 368 | GGATCACGACCTGCTGTGTC |
| transcript) | | | |

TABLE 2
Sequences of RNA strands used in studies
of LDL-R gene activation [UPDATE]

| | | Seq. | |
|---|---|---|---|
| Oligomer | strand | No. | Sequence (5′ - - - 3′) |
| LDLR+807 | sense | 122 | UUCCAGUGCUCUGAUGGAAdTdT |
| | antisense | 123 | UUCCAUCAGAGCACUGGAAdTdT |
| LDLR-75 | sense | 257 | AAAAUCACCCCACUGCAAAdTdT |
| | antisense | 258 | UUUGCAGUGGGGUGAUUUUdTdT |
| LDLR-68 | sense | 259 | CCCCACUGCAAACUCCUCCdTdT |
| | antisense | 260 | GGAGGAGUUUGCAGUGGGGdTdT |
| LDLR-65 | sense | 261 | CACUGCAAACUCCUCCCCCdTdT |
| | antisense | 262 | GGGGGAGGAGUUUGCAGUGdTdT |
| LDLR-59 | sense | 263 | AAACUCCUCCCCCUGCUAGdTdT |
| | antisense | 264 | CUAGCAGGGGGAGGAGUUUdTdT |
| LDLR-56 | sense | 265 | CUCCUCCCCCUGCUAGAAAdTdT |
| | antisense | 266 | UUUCUAGCAGGGGGAGGAGdTdT |
| LDLR-35 | sense | 267 | UCACAUUGAAAUGCUGUAAdTdT |
| | antisense | 268 | UUACAGCAUUUCAAUGUGAdTdT |
| LDLR-28 | sense | 269 | GAAAUGCUGUAAAUGACGUdTdT |
| | antisense | 270 | ACGUCAUUUACAGCAUUUCdTdT |
| LDLR-24 | sense | 271 | UGCUGUAAAUGACGUGGGCdTdT |
| | antisense | 272 | GCCCACGUCAUUUACAGCAdTdT |
| LDLR-21 | sense | 273 | UGUAAAUGACGUGGGCCCCdTdT |
| | antisense | 274 | GGGGCCCACGUCAUUUACAdTdT |
| LDLR-18 | sense | 275 | AAAUGACGUGGGCCCCGAGdTdT |
| | antisense | 276 | CUCGGGGCCCACGUCAUUUdTdT |
| LDLR-15 | sense | 277 | UGACGUGGGCCCCGAGUGCdTdT |
| | antisense | 278 | GCACUCGGGGCCCACGUCAdTdT |
| LDLR-11 | sense | 279 | GUGGGCCCCGAGUGCAAUCdTdT |
| | antisense | 280 | GAUUGCACUCGGGGCCCACdTdT |
| LDLR-9 | sense | 281 | GGGCCCCGAGUGCAAUCGCdTdT |
| | antisense | 282 | GCGAUUGCACUCGGGGCCCdTdT |
| LDLR-6 | sense | 283 | CCCCGAGUGCAAUCGCGGGdTdT |
| | antisense | 284 | CCCGCGAUUGCACUCGGGGdTdT |
| LDLRmm1 | sense | 285 | UGCUUUAACUGGCGUUGGCdTdT |
| | antisense | 286 | GCCAACGCCAGUUAAAGCAdTdT |
| LDLRmm2 | sense | 287 | GAACUGCGGUAACUGAAGUdTdT |
| | antisense | 288 | ACUUCAGUUACCGCAGUUCdTdT |
| LDLRmm3 | sense | 369 | UCCAGAAAAUGACGUGGGCdTdT |
| | antisense | 370 | GCCCACGUCAUUUUCUGGAdTdT |
| LDLRmm4 | sense | 371 | UGCUGUAAAUGAGGAGCGCdTdT |
| | antisense | 372 | GCGCUCCUCAUUUACAGCAdTdT |
| LDLRmm5 | sense | 373 | GAUAAGGUGUAAAUGACGUdTdT |
| | antisense | 374 | ACGUCAUUUACACCUUAUCdTdT |
| LDLRmm6 | sense | 375 | GAAAUGCUGUAAUUCACCUdTdT |
| | antisense | 376 | AGGUGAAUUACAGCAUUUCdTdT |
| Scrl | sense | 289 | GAGAUUACGAUUGCUGGGCdTdT |
| | antisense | 290 | GCCCAGCAAUCGUAAUCUCdTdT |
| Scr2 | sense | 291 | GAAUCGCUUAGAUUAAGAGdTdT |
| | antisense | 292 | CUCUUAAUCUAAGCGAUUCdTdT |
| Scr3 | sense | 293 | UCGUCAGUGGAGUCAGAGUdTdT |
| | antisense | 294 | ACUCUGACUCCACUGACGAdTdT |
| Scr4 | sense | 295 | GUGGAUCUCACGGUGUAGAdTdT |
| | antisense | 296 | UCUACACCGUGAGAUCCACdTdT |
| Scr5 | sense | 297 | UAGCUAGCUAGUAGAUAAGdTdT |
| | antisense | 298 | CUUAUCUACUAGCUAGCUAdTdT |
| LDLR-24 | sense | 299 | uGcuGuAAAuGAcGuGGGcdTdT |
| (2′-O-methyl) | antisense | 300 | GcccAcGuCAuuuAcAGcAdTdT |
| LDLR-24 | sense | 301 | UGCUGUAAAUGACGUGGGCdTdT |
| (2′-fluoro) | antisense | 302 | GCCCACGUCAUUUACAGCAdTdT |
| LDLR-28 | sense | 303 | GAAAuGcuGuAAAuGAcGudTdT |
| (2′-O-methyl) | antisense | 304 | AcGucAuuuAcAGcAuuucdTdT |
| LDLR-28 | sense | 305 | GAAAUGCUGUAAAUGACGUdTdT |
| (2′-fluoro) | antisense | 306 | ACGUCAUUUACAGCAUUUCdTdT |

Mismatch bases are underlined.
dT represents deoxythymidine.
2′-O-methyl modified nucleotides are shown in small letters.
2′-fluoro modified nucleotides are shown in italic letters.

Quantitative Reverse Transcription-PCR (qRT-PCR). Total RNA was extracted using TRIzol (Invitrogen). RNA samples were treated with DNase I (Worthington Biochemical) at 25° C. for 10 min and reverse transcription was performed using High Capacity Reverse Transcription Kit (Applied Biosystems) according to the manufacturer's protocol. Quantitative PCR (qPCR) was performed on a 7500 real-time PCR system (Applied Biosystems) using iTaq SYBR Green Supermix (Bio-Rad). Primer sequences are described in Table 1. Standard curves for each primer set were made to evaluate primer efficiency in PCR amplification. qPCR data for comparing expression levels of LDLR mRNA and the antisense transcript were normalized by the difference in primer efficiency.

Cell culture and transfection. Unmodified, 2′-O-methyl, and 2′-fluoro RNAs with two 2′-deoxythymidine bases at the 3′ end were obtained from Integrated DNA Technologies or Alnylam Pharmaceuticals. HepG2 (American Type Culture Collection (ATCC)) and fibroblast cells (GM04281; Coriell) were cultured with Minimum Essential Medium Eagle (MEM; Sigma) supplemented with 10% FBS, 1% MEM non-essential amino acids (Sigma), and 1 mM sodium pyruvate (Sigma). HuH-7 (Japanese Collection of Research Bioresources) and SW480 cells (ATCC) were cultured with Dulbecco's Modified Eagle's Medium (Sigma) supplemented with 10% FBS and 1 mM sodium pyruvate. Cells were plated in 6-well plates at 120,000 (HepG2 and HuH-7), 60,000 (fibroblast), or 150,000 (SW480) cells/well 2 days before transfection. Duplex RNAs were transfected into cells using Lipofectamine RNAiMAX (Invitrogen). Cationic lipid (2.4 μL for 50 nM dsRNA) was added to OptiMEM (Invitrogen) containing oligonucleotides and the oligonucleotide-lipid mixture (250 μL) was incubated at room temperature for 20 min. OptiMEM (for HepG2 and fibroblast) or full media (for HuH-7 and SW480) was added to a final volume of 1.25 mL and the mixture was applied to cells. Media was exchanged 1 day later with fresh supplemented media (2 mL).

Chromatin Immunoprecipitation (ChIP)/RNA Immunoprecipitation (RIP). HepG2 cells were seeded at 1,080,000 cells in 15 cm dishes 2 days before transfection for ChIP or RIP experiments. Two dishes were treated with activating agRNAs (LDLR-24(U/U) and LDLR-28(U/U)) or mismatch controls (LDLRmm1, LDLRmm3, and LDLRmm4) (50 nM). Four days after transfection, cells were crosslinked with 1% formaldehyde. Cells were recovered by scraping and nuclei were isolated using hypotonic lysis buffer (5 mL; 10 mM Tris-HCl (pH 7.5), 10 mM NaCl, 3 mM MgCl₂, 0.5% NP-40). Nuclei were lysed in lysis buffer (1 mL; 1% SDS, 10 mM EDTA, 50 mM Tris-HCl (pH 8.1), 1× Roche protease inhibitors cocktail, 40 U/mL RNasin Plus RNase Inhibitor (Promega)) and sonicated (2 pulses, 20% power, 20 sec).

The cell lysate (100 μL) was incubated overnight with antibodies in immunoprecipitation buffer (1 mL; 0.01% SDS, 1.1% Triton-X-100, 1.2 mM EDTA, 16.7 mM Tris-HCl (pH 8.1), 167 mM NaCl, and 1× Roche protease inhibitors cocktail, 40 U/mL RNasin Plus RNase Inhibitor). Monoclonal anti-RNAP II (2 μg; Millipore) and polyclonal anti-H3K27me3 (2 μg; Millipore) antibodies were used for ChIP experiments. Polyclonal anti-AGO1 (2 μg; Millipore) and polyclonal anti-AGO2 (2 μg; Millipore) antibodies were used for RIP experiments. Normal mouse IgG (2 μg; Millipore) or normal rabbit IgG (2 μg; Millipore) was used as a control. After the antibodies were recovered with 50 μL of Protein G Plus/Protein A Agarose Beads (Calbiochem), the beads were washed with 1 mL of low salt (0.1% SDS, 1% Triton-X-100, 2 mM EDTA, 20 mM Tris-HCl (pH 8.1), 150 mM NaCl), high salt (see low salt but with 500 mM NaCl), LiCl solution (0.25 M LiCl, 1% NP-40, 1% deoxycholate, 1 mM EDTA, and 10 mM Tris-HCl (pH 8.1)), and TE buffer (pH 8.0). Protein was eluted twice with 250 μL of elution buffer (1% SDS, 0.1M NaHCO₃, and 40 U/mL RNasin Plus RNase Inhibitor) for 15 minutes at room temperature. Crosslinking was reversed by adding NaCl to 200 mM and heating at 65° C. for at least 2 hours. Protein was digested by incubating with Proteinase K (1 μg/mL; Invitrogen) at 42° C. for 50 min, followed by phenol extraction using an equal volume of phenol:chloroform:isoamyl alcohol. DNA/RNA in the aqueous layer was precipitated using 1/10 volume sodium acetate, 2.2 volumes ethanol, and glycogen (40 μg; Sigma). For ChIP, the pellet was resuspended in 80 μL of nuclease-free water. qPCR was performed using iTaq SYBR Supermix and primers specific for the LDLR promoter (5′-CCTGCTAGAAACCTCACATTG-3′ (SEQ ID NO:367); 5′-GGATCACGACCTGCTGTGTC-3′) (SEQ ID NO:368). For RIP, the pellet was resuspended in 16 μL of nuclease-free water. After treating each sample with DNase I at 25° C. for 10 min, reverse transcription reactions were performed only for input and +RT samples. qPCR was performed using iTaq SYBR Supermix and primers specific for the antisense transcript. PCR products were analyzed on 2.5% agarose gel and stained with ethidium bromide.

Analysis of LDLR protein expression. Cells were harvested 4 days after transfection for western blotting analysis. Cells were detached from plates using cell dissociation solution (Sigma) and lysed with lysis buffer (50 mM Tris-HCl, 120 mM NaCl, 0.5% NP-40, 1 mM EDTA, 1 mM DTT, and protease inhibitor (Calbiochem)). Protein concentrations were quantified with BCA assay kit (Thermo Scientific). SDS-PAGE was performed using 7.5% Tris-HCl gels (Bio-Rad). Gels were run at 100 V for 60 min. After gel electrophoresis, proteins were transferred to nitrocellulose membrane (Hybond-C-Extra; GE Healthcare) at 100 V for 2 h. After blocking the membrane with 5% non-fat dry milk/TBST at room temperature for 1 h, the membrane was incubated with primary antibody specific for LDLR or β-actin at the following dilution ratio: anti-LDLR antibody (ab52818; 1:10,000; abcam), anti-β-actin antibody (1:20,000; Sigma). HRP-conjugated anti-rabbit (1:10,000; Jackson ImmunoResearch) or anti-mouse (1:20,000; Sigma) secondary antibody was used for visualizing proteins using SuperSignal West Pico Chemiluminescent Substrate (Thermo Scientific). Protein bands were quantified using ImageJ software.

LDL Binding Assay. agRNAs (50 nM) were transfected in HepG2 cells as described above (Day 0). On day 4, cells were washed with cold PBS three times and then incubated with DiI-LDL (12 μg/mL; Invitrogen) or DiI-LDL (12 μg/mL)+unlabeled LDL (120 μg/mL; Invitrogen) in serum-free MEM at 4° C. for 2 h. After the incubation, cells were washed with cold PBS five times and then treated with 4% paraformaldehyde at room temperature for 25 min. After the fixation, cells were washed with PBS twice. Cells were observed using fluorescence microscopy (Zeiss Axiovert 200 M).

agRNAs (LDLR-24(U/U), LDLR-28(U/U), and LDLRmm1; 0, 25, 50, 100 nM) were also transfected into HepG2 cells for flow cytometry experiments. Four days after transfection, cells were harvested using cell dissociation solution and washed with 1 mL of PBS. After filtering cells using cell strainers (40 μm; BD Falcon), 250,000 cells in 250 μL of serum-free MEM were incubated with DiI-LDL (3 μg) at 4° C. for 2 h. Cells were collected by centrifugation (2500 rpm, 5 min) and then washed three times with 1 mL of PBS containing 0.5% BSA and 0.02% sodium azide. The fluorescence of cell-associated DiI-LDL was measured by FACScan (Beckton Dickinson) with 10,000 cells per sample.

Analysis of Interferon Responsive Genes. mRNA levels of the interferon responsive genes including OAST, OAS2, MX1, IFITM1, and ISGF3γ were measured by qRT-PCR. agRNAs (50 nM) and Poly I:C (0-100 ng/mL; Sigma) were transfected into HepG2 cells using the cationic lipid as described above. Three days after transfection, total RNAs from dsRNA-treated, poly I:C-treated, or untreated samples were isolated using TRIzol. The RNAs were treated with DNase I at 25° C. for 10 min, followed by reverse transcription reaction at 37° C. for 2 h. qPCR was performed using iTaq SYBR Supermix and primers specific for the interferon responsive genes (Interferon Response Detection Kit; System Biosciences). LDLR protein levels on Day 4 were also measured by western blot analysis.

Combination Treatment with Lovastatin and agRNAs. Inactive lovastatin (17 mg; Sigma) in the lactone form was converted into its active form as previously described (Morimoto et al., 2006). The stock solution (5 mM in 5% EtOH) was stored at −80° C. until use. dsRNAs (50 nM) were transfected into HepG2 cells as described above (Day 0), and the media were exchanged one day later. Two days after transfection, lovastatin (10 or 30 μM) or 5% EtOH solution (vehicle) was added to each dsRNA-treated cell (final EtOH concentration: 0.03%). The cells were harvested on Day 4 for western blot analysis.

Combination treatment with 25-hydroxycholesterol and agRNAs. dsRNAs (50 nM) were transfected into HepG2 cells as described above (Day 0), and the media were exchanged one day later. Two days after transfection, 25-hydroxycholesterol (2 μM; Sigma) dissolved in EtOH or EtOH only (vehicle) was added to each dsRNA-treated cell (final EtOH concentration: 0.04%). The cells were harvested on day 4 for western blot analysis.

### Example 2

### Results

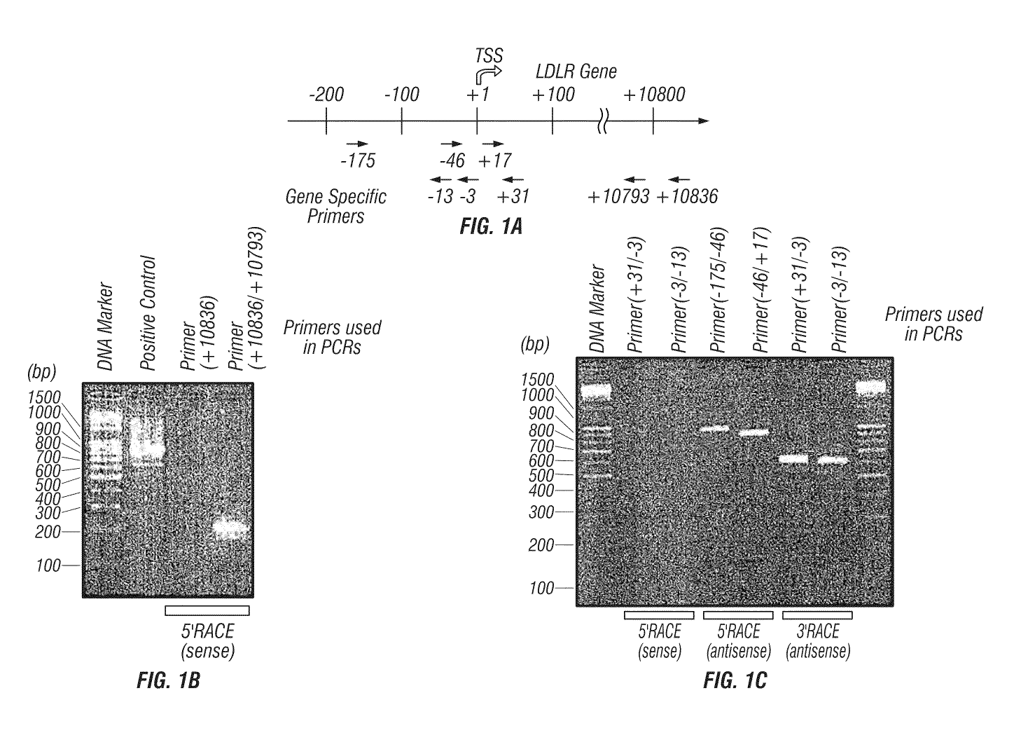

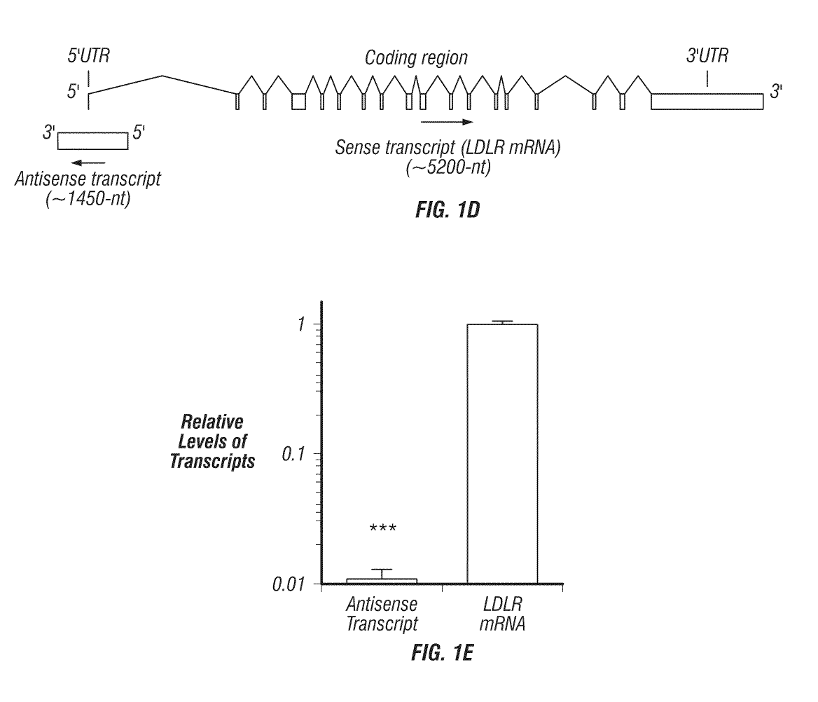

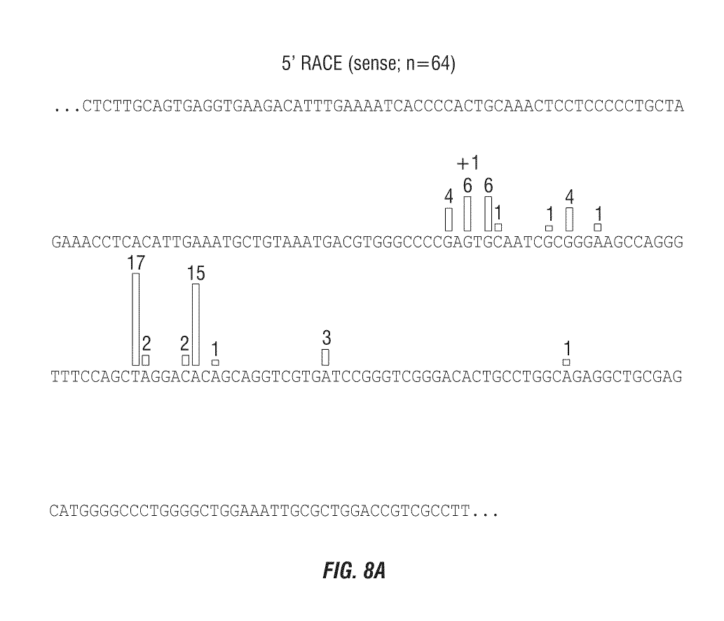

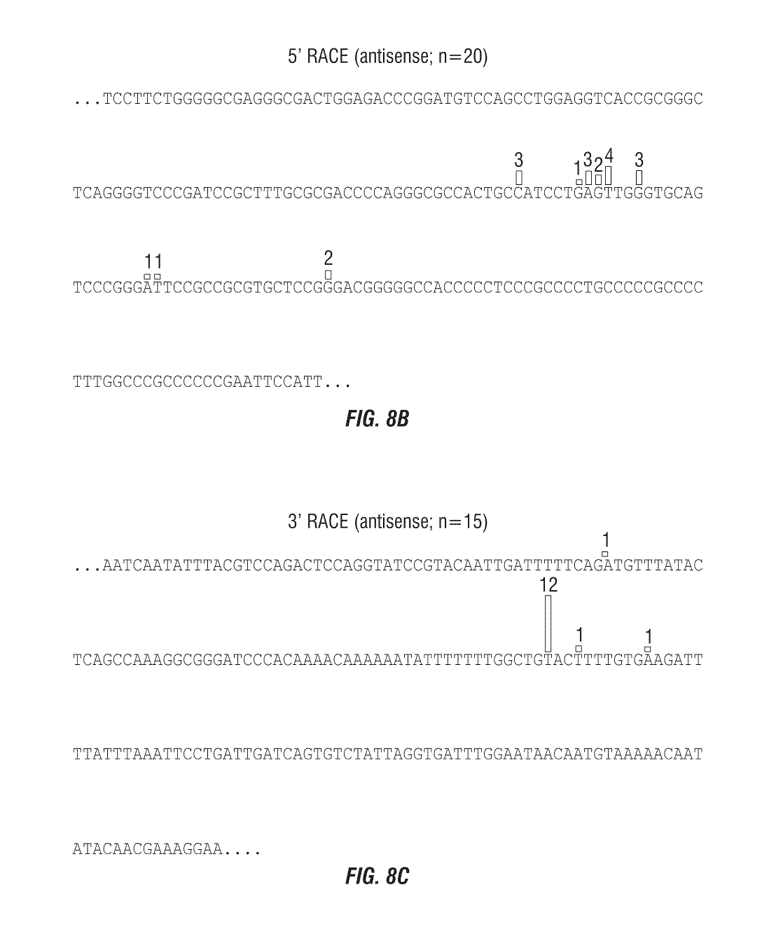

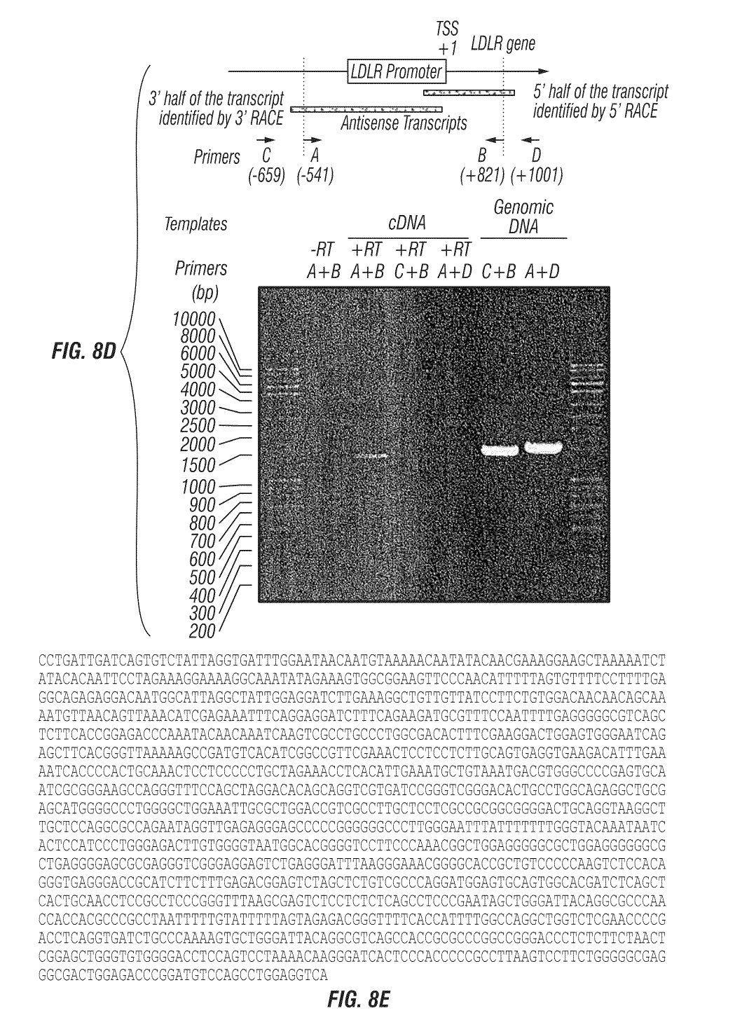

Characterization of transcripts at the LDLR promoter. Designing RNAs to target gene promoters requires an accurate identification of the transcription start site. The inventors used Rapid Amplification of cDNA Ends (RACE) to analyze start sites for LDLR mRNA in HepG2 cultured human liver cells (FIGS. 1A-B, and Table 1). After sequencing 69 clones, the inventors identified 14 transcription start sites for LDLR mRNA, and the +1 transcription start site was designated based on the 5′ RACE analysis and 5′ EST data from the database for transcription start sites (DBTSS: dbtss.hgc.jp/) (FIG. 8A).

In previous studies of agRNA-mediated modulation of gene expression, the inventors examined expression of progesterone receptor (PR). The inventors observed that, rather than recognize chromosomal DNA, agRNAs recognize noncoding transcripts that overlap the PR gene promoter (Schwartz et al., 2008). The noncoding transcript at the PR promoter was an antisense transcript synthesized in a direction opposite to that of PR mRNA.

To investigate whether noncoding transcripts are expressed in the LDLR promoter, the inventors performed 5′ and 3′ RACE using LDLR promoter-specific primers. The inventors discovered a 1450-nt antisense transcript that overlaps the LDLR promoter, initiating at ˜+880 and terminating at ˜−570 (FIGS. 1C-D; FIGS. 8B-E). This transcript is polyadenylated, unspliced, and expressed at levels approximately 90-fold below LDLR mRNA (FIG. 1E). The inventors did not detect sense transcripts overlapping the LDLR promoter, making the antisense transcript the most plausible target for anti-LDLR agRNAs.

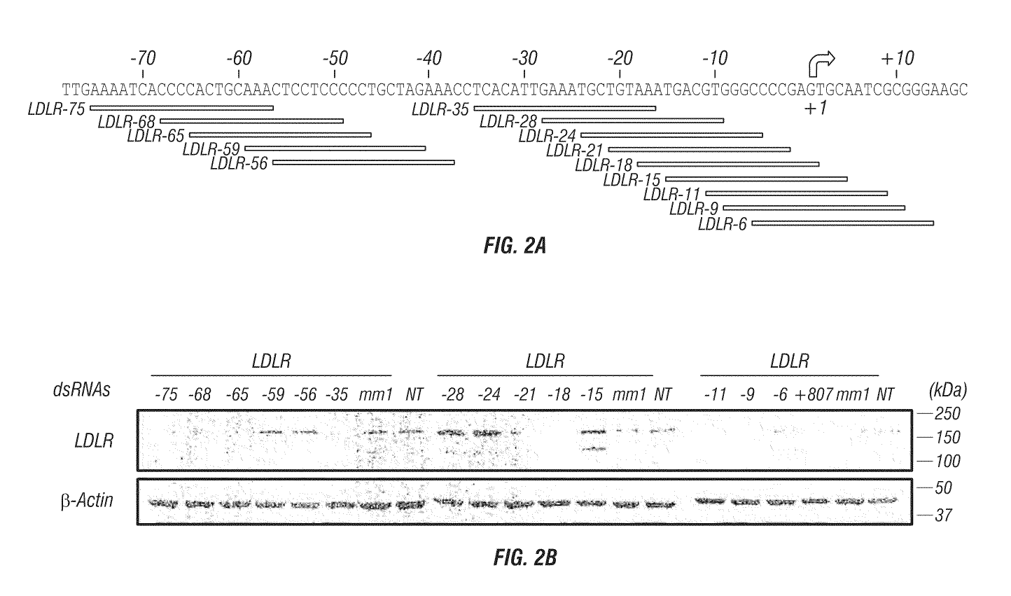

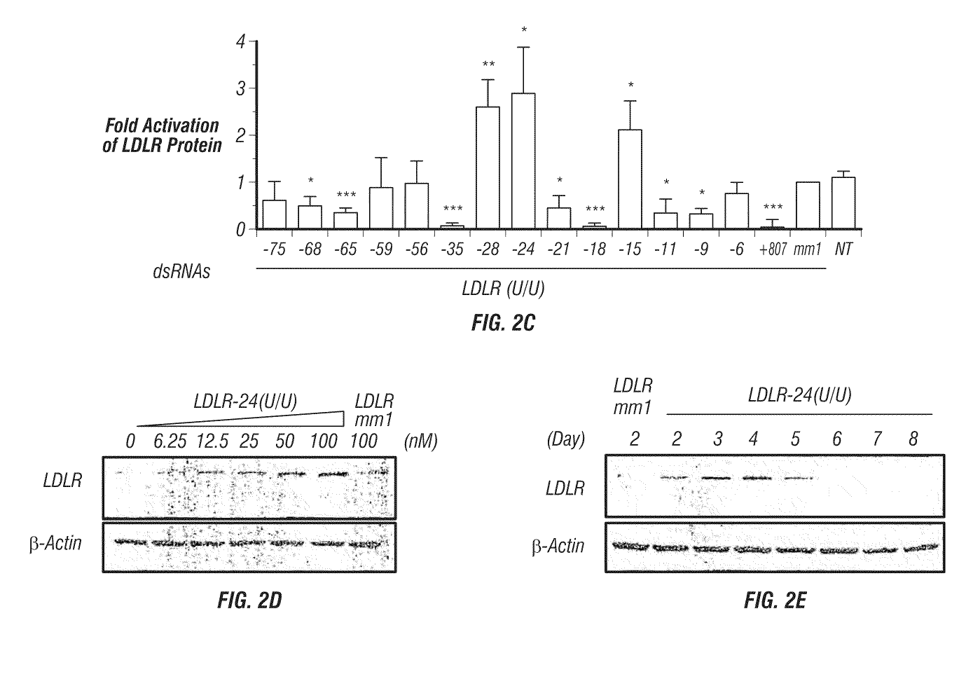

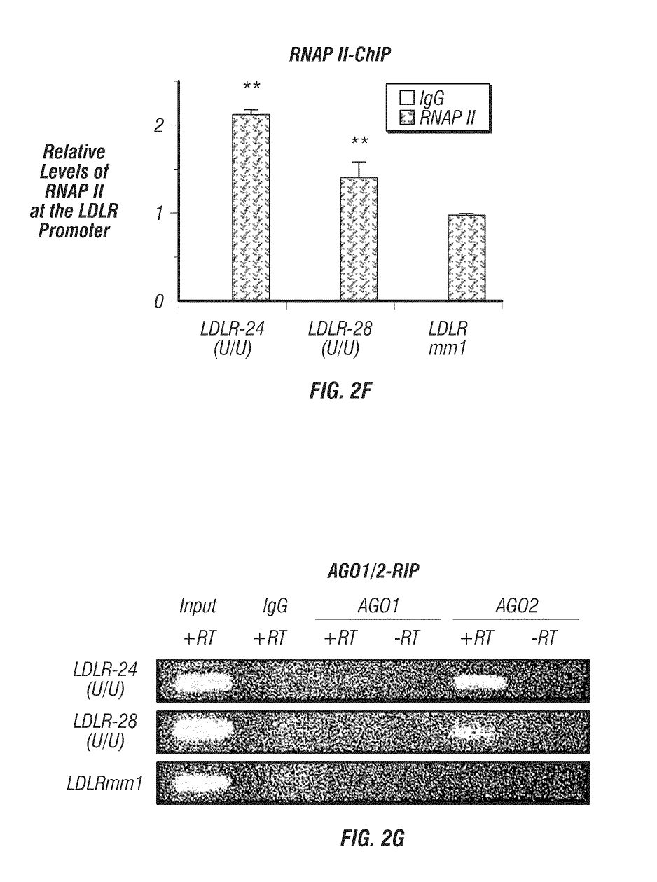

Design of agRNAs. The agRNAs used in these studies were 19-base pair RNA duplexes with 2-base deoxythymidine overhangs at the 3′ ends (Table 2). The agRNAs were designed to be complementary to sequences throughout the promoter for LDLR (FIG. 2A). agRNA nomenclature is defined by the most upstream base. For example, LDLR-24 would target bases −24 to −5 relative to the +1 transcription start site for LDLR. LDLR+807 is a siRNA complementary to LDLR mRNA. It represses LDLR expression through the standard post-transcriptional RNAi mechanism and the inventors used it as a positive control for evaluating transfection efficiency. Mismatch-containing dsRNAs LDLRmm1 and LDLRmm2 were designed based on the sequence of LDLR-24 and LDLR-28, respectively.

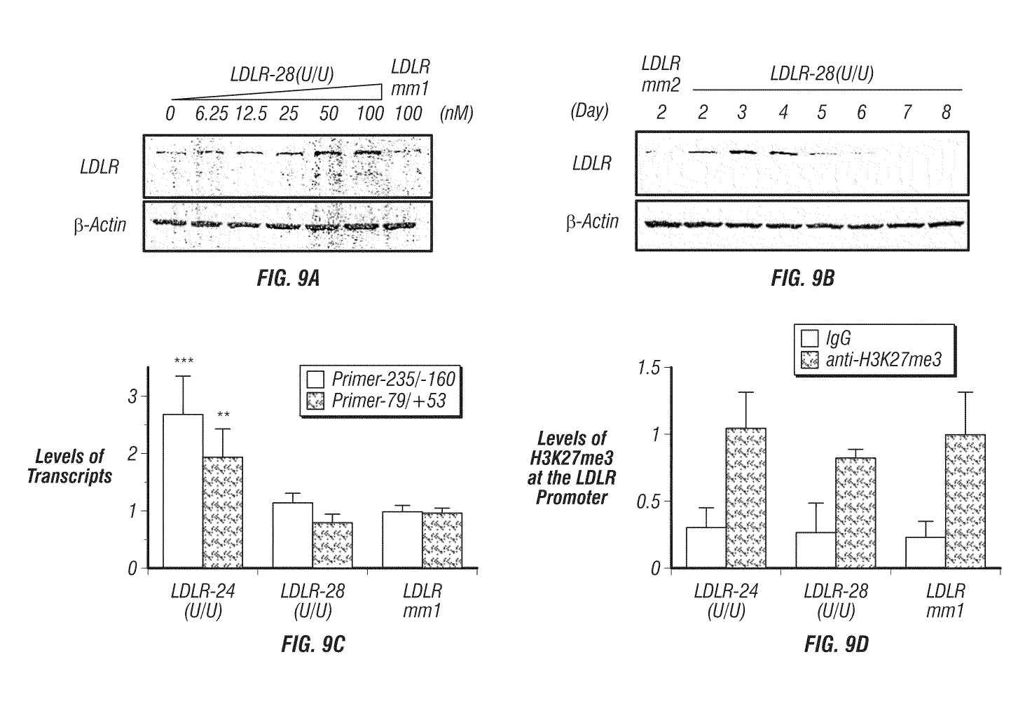

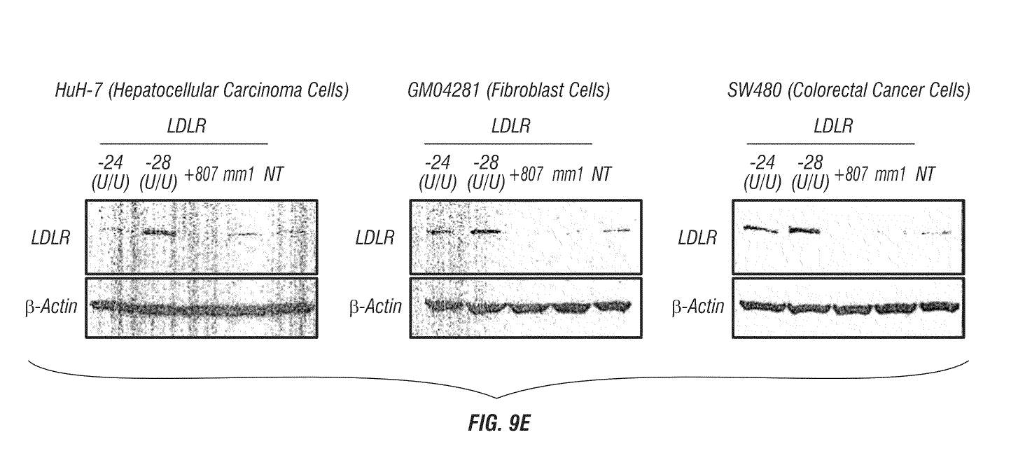

Activation of LDLR Expression by agRNAs. The inventors transfected agRNAs into HepG2 cells and evaluated expression of LDLR protein by western blotting four days later. RNAs were transfected at 50 nM, a concentration chosen to combine maximal efficacy with minimal toxicity to cells. Western analysis revealed two immunoreactive bands due to the precursor and mature forms of LDLR described above. agRNAs LDLR-24, LDLR-28, and LDLR-15 increased LDLR protein levels by 2-3 fold (FIGS. 2B-C). Enhanced expression was dose dependent and transient, reaching a maximum level four days after transfection (FIGS. 2D-E; FIGS. 9A-B). Activation of LDLR expression by LDLR-24 and LDLR-28 was characterized by potencies (EC₅₀) values of 26 and 16 nM respectively (FIG. 12).

Consistent with the gene activation at the level of protein, chromatin immunoprecipitation (ChIP) revealed 1.5 to 2 fold elevation of levels of RNA polymerase II (RNAP II) at the LDLR promoter (FIG. 2F). Levels of the antisense transcript did not decrease after transfection of activating agRNAs (FIG. 9C), suggesting that cleavage of the transcript by AGO2 doesn't appear to be a primary cause of the activation. The inventors also monitored levels of H3K27 trimethylation (H3K27me3), which is a transcription-suppressive chromatin mark. Unlike the inventors' previous observations in activating agRNAs for PR (Yue et al., 2010), no significant changes were detected for the chromatin mark (FIG. 9D). This might reflect that H3K27me3 is not a dominant regulatory factor for LDLR gene in HepG2 cells where basal expression level of the gene is relatively high.

To check cell specificity of LDLR activation by agRNAs, LDLR-24 and LDLR-28 were also tested in three other cell lines including HuH-7, fibroblast cells (GM04281), and SW480. The inventors observed a similar effect of the oligomers on LDLR expression in the cell lines except for LDLR-24 in HuH-7 cells (FIG. 9E).

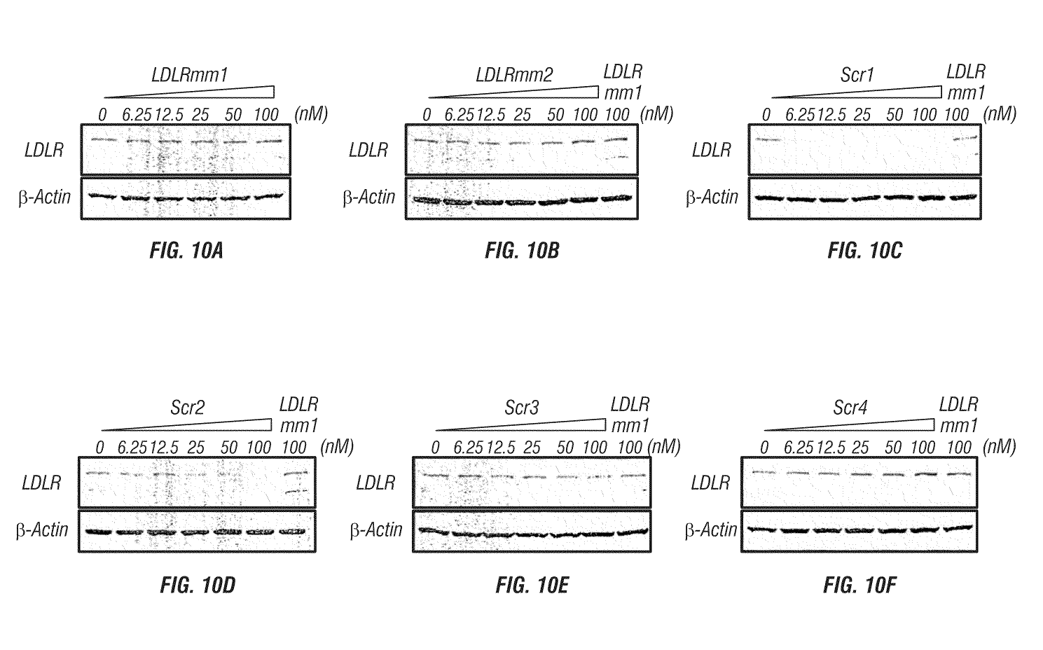

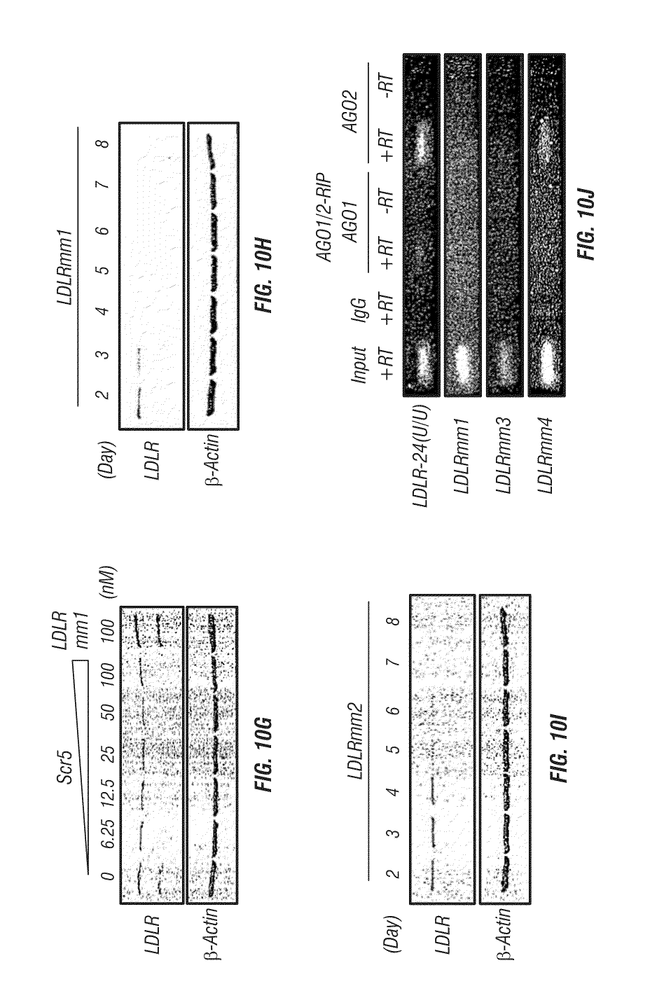

When mismatch duplex RNAs were added, LDLR expression started to decrease 4-5 days after transfection (FIGS. 10H-I), probably due to a cellular response to the conditions where cholesterol is less required as cells become confluent. Thus, the activation the inventors observe runs counter to a natural tendency of LDLR expression to decrease over time.

There are four AGO proteins in mammalian cells (Siomi and Siomi, 2009). AGO2 is the “catalytic engine” that drives mRNA cleavage (Liu et al. 2004; Meister et al. 2004; Rand et al. 2004), while the roles of AGO1, AGO3, and AGO4 are less well known. The inventors and others have previously reported that the action of promoter-targeted RNAs involves AGO1 or AGO2 (Li et al., 2006; Kim et al., 2006; Janowski et al., 2006; Morris et al., 2008; Napoli et al., 2009; Chu et al. 2010; Yue et al. 2010).

To determine whether AGO proteins might also be involved in agRNA-mediated activation of LDLR, the inventors performed RNA immunoprecipitation (RIP) for AGO1 and AGO2 upon addition of agRNAs. Using RIP the inventors observed primary recruitment of AGO2 to the LDLR antisense transcript in cells treated with LDLR-24(U/U) or LDLR-28(U/U) (FIG. 2G). Recruitment of AGO1 could also be detected but at lower levels. No PCR products were amplified in the samples without reverse transcription, suggesting that the inventors were not detecting amplification of chromosomal DNA.

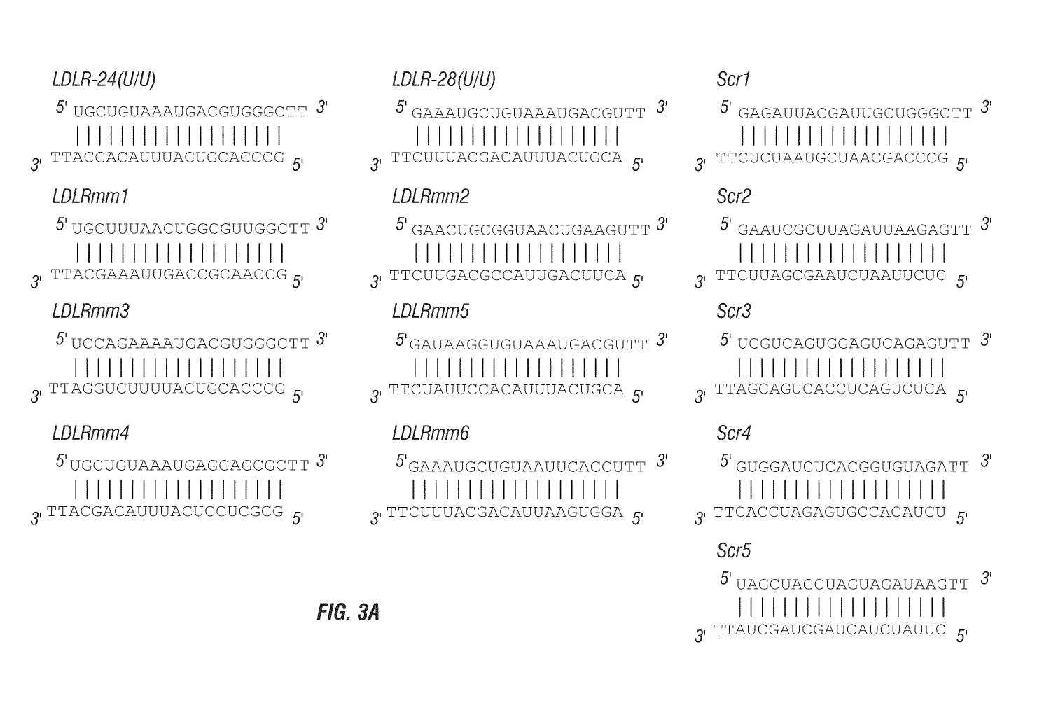

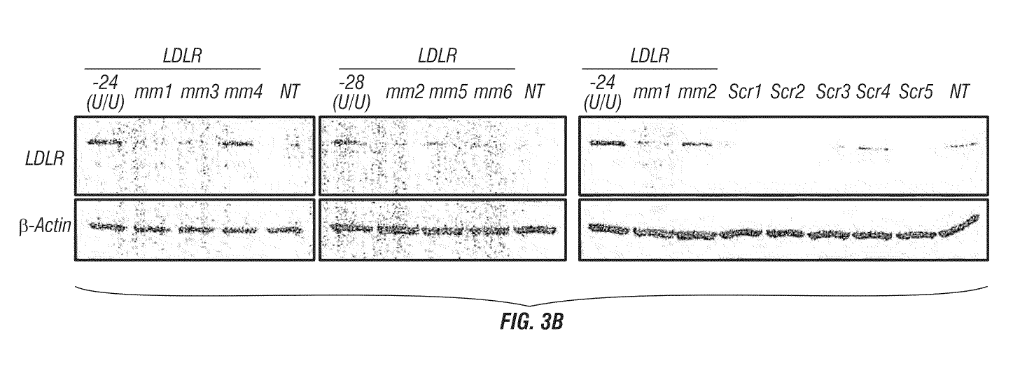

Testing Mismatch-containing or Randomly Scrambled Oligomers. To evaluate whether sequence complementarity of agRNA to the LDLR promoter is required for activation, the inventors tested another nine mismatch-containing or randomly scrambled RNA duplexes based on the sequence of LDLR-24 or LDLR-28 in addition to LDLRmm1 and LDLRmm2 (FIG. 3A and Table 2). Mismatch-containing RNAs were designed to spread mismatches throughout the RNA or concentrate them in regions with potential seed sequences. Seed sequences contain positions 2-8 within the duplex RNA and complementarity between seed sequences and RNA targets is known to be an important determinant for successful RNAi.

With one exception, these control oligomers did not activate LDLR expression (FIG. 3B and FIG. 10). The exception was LDLRmm4 which contains three mismatches outside the seed sequence predicted for recognition of the antisense transcript. One explanation for activation by LDLRmm4 is that it preserves the potential to form necessary seed sequence interactions with the antisense transcript detected at the LDLR promoter. Consistent with this hypothesis, RIP experiments for the mismatch oligomers showed recruitment of AGO2 to the antisense transcript by active duplex LDLRmm4 that contained mismatches outside the seed sequence, but not by inactive duplex LDLRmm3 that contained mismatches disrupting the predicted seed sequence (FIG. 10J).

Several RNA duplexes, notably LDLR-65, LDLR-35, and LDLR-18, appeared to reduce gene expression (FIGS. 2B-C). However, the inventors observed that some of the scrambled oligomers induced non-sequence-specific silencing of LDLR gene (FIG. 10), complicating interpretation of LDLR gene silencing by agRNAs. Because of the tendency towards nonspecific silencing and the inventors' focus on gene activation, the inventors did not investigate gene silencing further.

Effect of Chemical Modifications on Activation of LDLR. Development of duplex RNAs as drugs will require chemical modifications to improve their stability, specificity, and potency (De Paula et al., 2007; Watts et al., 2008). Modifying siRNAs can reduce off-target effects resulting from the miRNA pathway (Jackson et al., 2006), the innate immune system (Judge and MacLachlan, 2008), or loading of the wrong strand (Bramsen et al., 2007).

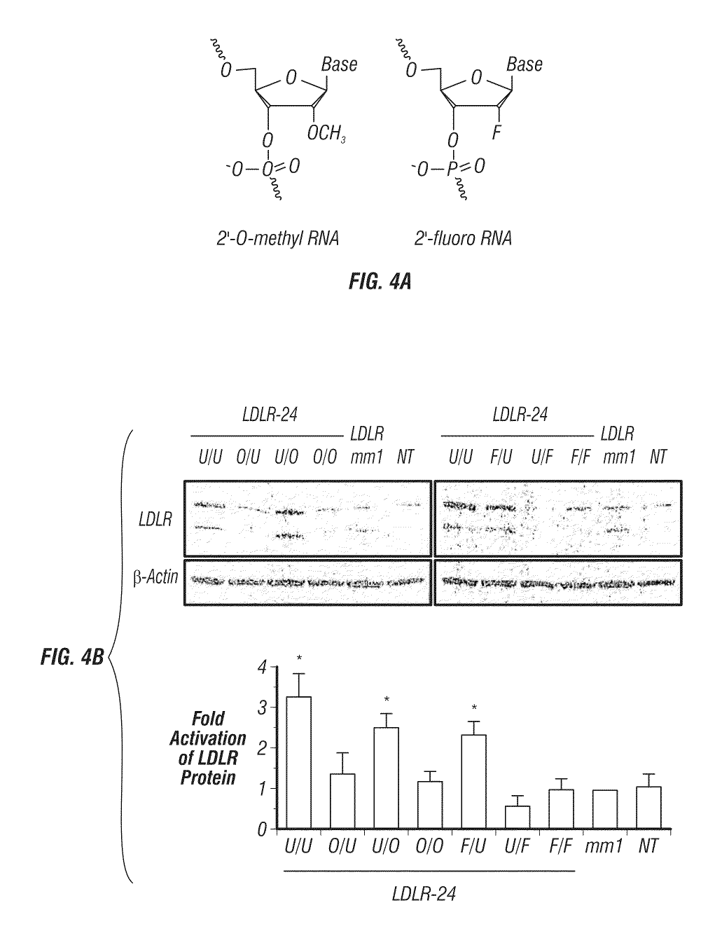

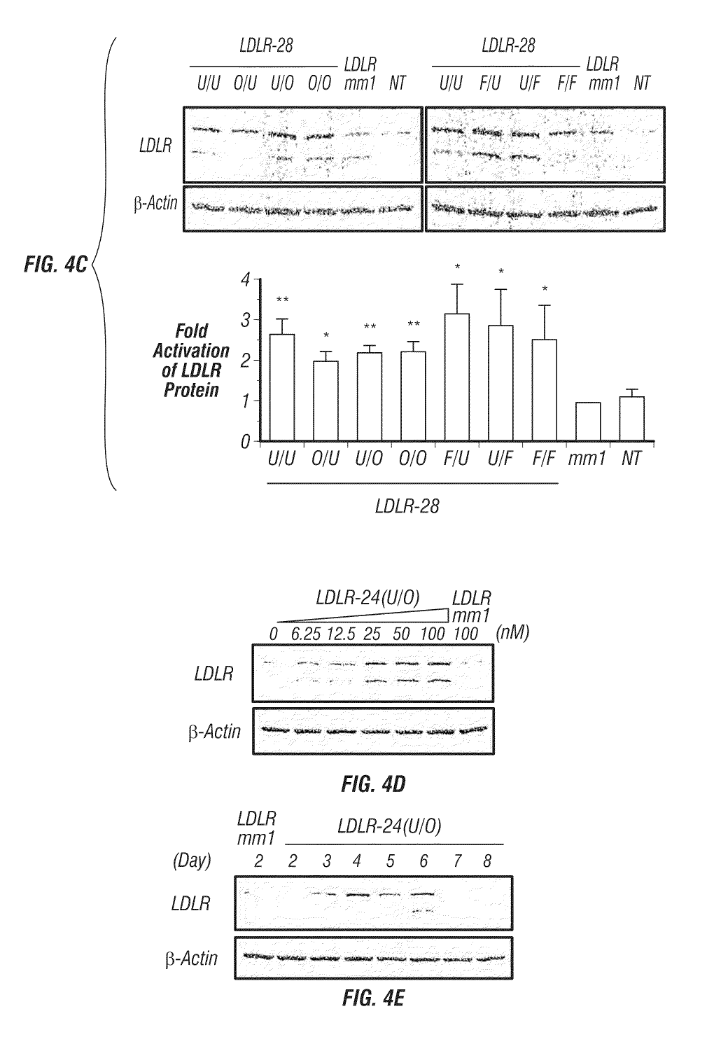

To determine whether activation of LDLR would be compatible with chemical modifications commonly used during drug development, the inventors tested introducing 2′-β-methyl or 2′-fluoro nucleotides into LDLR-24 or LDLR-28 (FIG. 4A and Table 2). Each type of modified duplex is assigned two uppercase letters. The first letter describes the chemical modification of the sense strand, while the second letter describes modification of the antisense strand. For example, U/F would have an unmodified sense strand and an antisense strand containing 2′-fluoro substitutions.

We observed activation of LDLR expression by chemically modified duplexes containing 2′-O-methyl or 2′-fluoro RNA (FIGS. 4B-C). Potencies (EC₅₀ values) ranged from 4.1 to 38 nM (FIG. 12). Maximal activation (Aₘₐₓ) was between 2.2 and 3.3-fold. For LDLR-24, activation was achieved with 2′-O-methyl RNA on the antisense strand or with 2′-fluoro RNA on the sense strand. When variants of LDLR-28 were tested, activation was observed regardless of whether the 2′-O-methyl or 2′-fluoro modifications were on the sense or antisense strand. The phenomenon that similar patterns of chemical modification have different effects on gene activation when applied to different sequences has been observed previously in chemically modified agRNAs that activate PR expression (Watts et al., 2010).

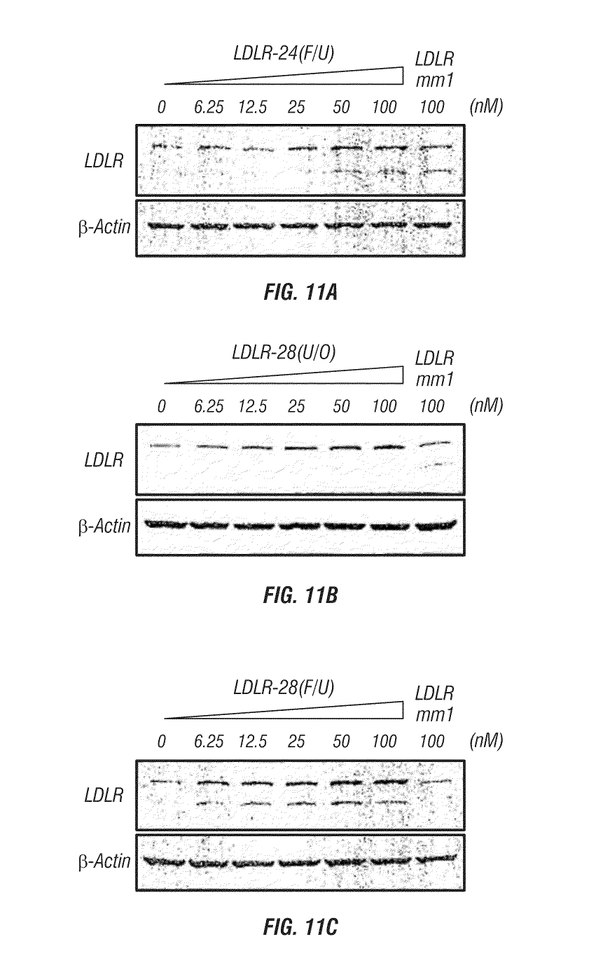

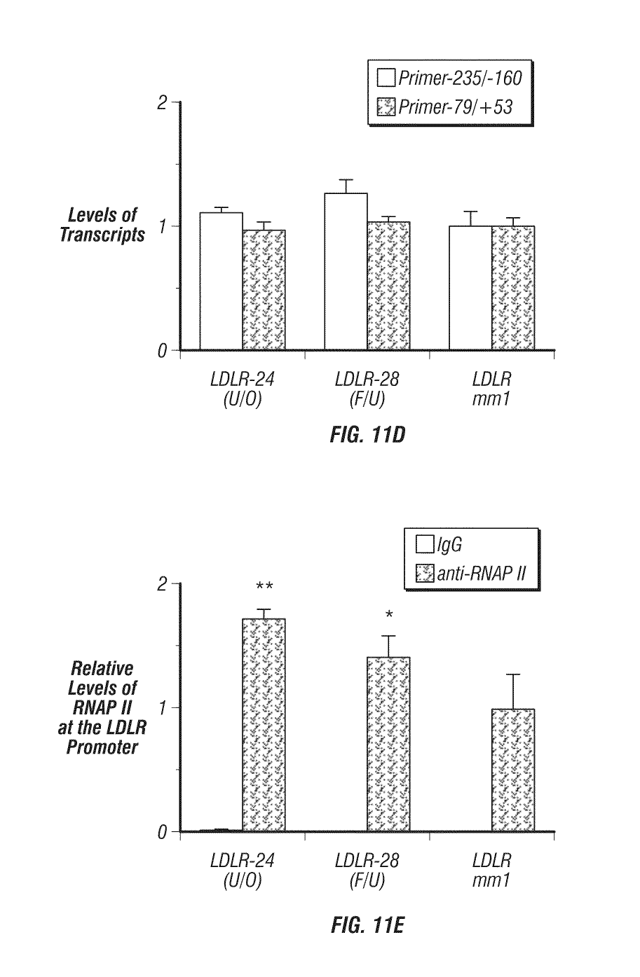

The dependence of activation on the concentration of agRNA duplex was similar regardless of which modified agRNA was used (LDLR-24(U/O), LDLR-24(F/U), LDLR-28(U/O), or LDLR-28(F/U)) (FIG. 4D and FIGS. 11A-C). Relative to unmodified LDLR-24(U/U) (FIG. 2E), activation of LDLR by modified LDLR-24(U/O) persisted for a longer period, with elevated protein levels being observed until Day 6 after transfection (FIG. 4E). These data demonstrate that agRNA-mediated activation of LDLR expression is compatible with chemical modifications commonly used during development of duplex RNA therapeutics.

Similar to the results for unmodified agRNAs, the inventors did not observe any significant changes in the antisense transcript levels after treatment with chemically modified agRNAs (FIG. 11D). In ChIP experiments for RNAP II, ˜1.5-fold increase of RNAP II was observed at the LDLR promoter (FIG. 11E). These results suggest that mechanism of the LDLR activation is conserved between unmodified and modified oligomers.

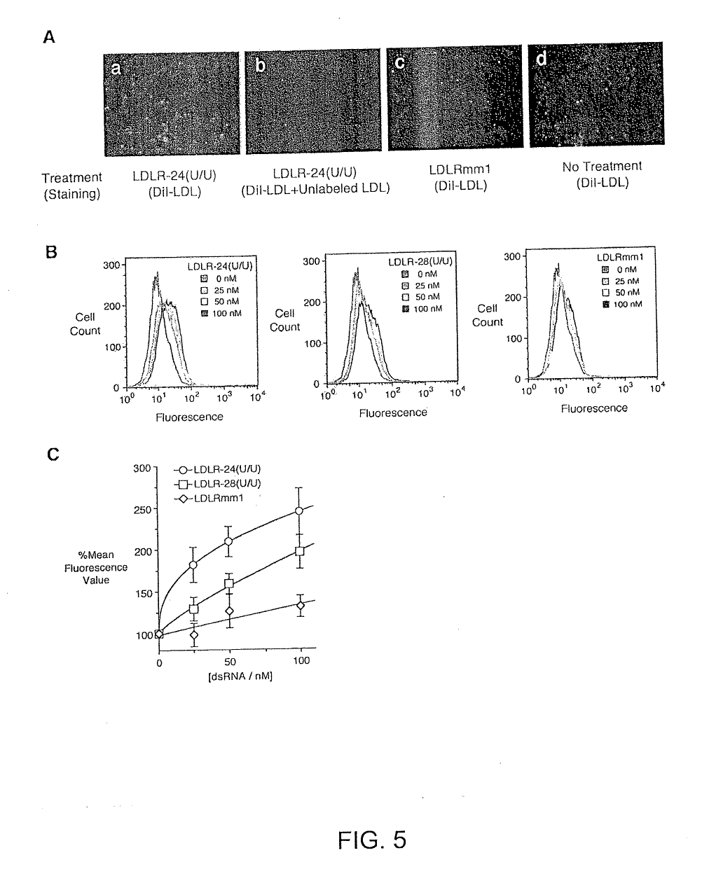

Upregulation of Cell-Surface LDLR. To examine whether agRNA-mediated activation of LDLR expression would lead to enhanced display of LDLR on the cell-surface and greater binding of LDL particles to the receptors, the inventors performed LDL binding assay using (3,3′-dioctadecylindocarbocyanine)-labeled LDL (DiI-LDL). After treating cells with an activating agRNA or a mismatch control, the cells were incubated with DiI-LDL and binding of DiI-LDL to the cell-surface was measured by fluorescence microscopy. The inventors observed increased fluorescence in cells treated with Dil-LDL after addition of LDLR-24(U/U) relative to cells treated with the mismatch control LDLRmm1 (FIG. 5A). Addition of unlabeled LDL quenched the fluorescence, indicating that the interaction is specific.

Binding of DiI-LDL to the cell-surface was quantified using flow cytometry. Cells treated with varying concentrations of activating agRNAs or a mismatch control were incubated with DiI-LDL and fluorescence from DiI-LDL bound to the cell-surface was measured. The inventors observed enhanced fluorescence from DiI-LDL in LDLR-24(U/U)- or LDLR-28(U/U)-treated cells relative to LDLRmm1-treated cells in a dose-dependent manner (FIGS. 5B-C). These results indicate that upregulation of LDLR by agRNAs led to enhanced trafficking of LDL particles to cell surface.

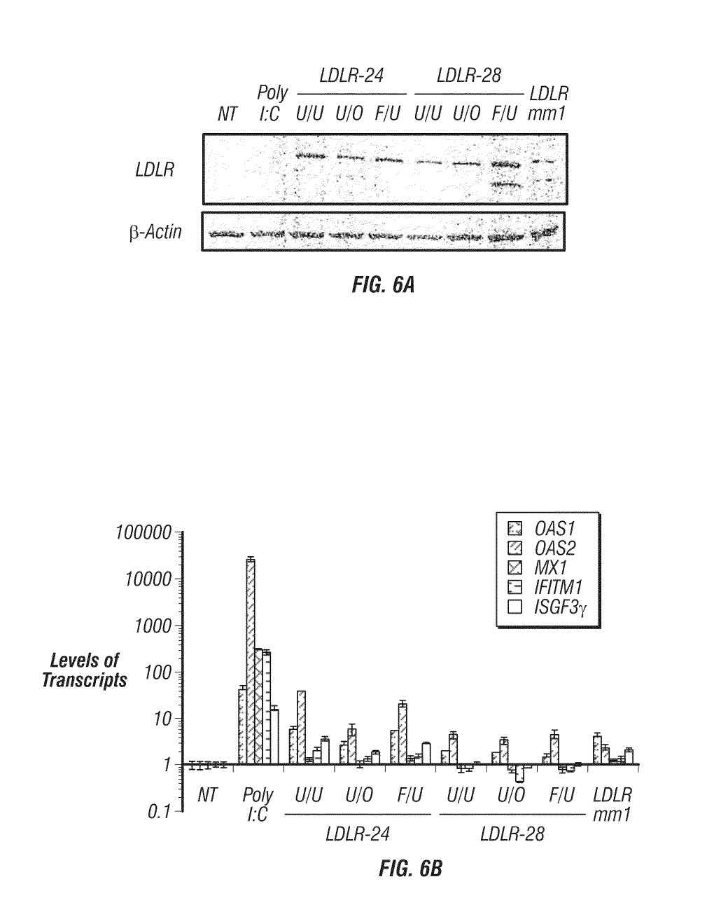

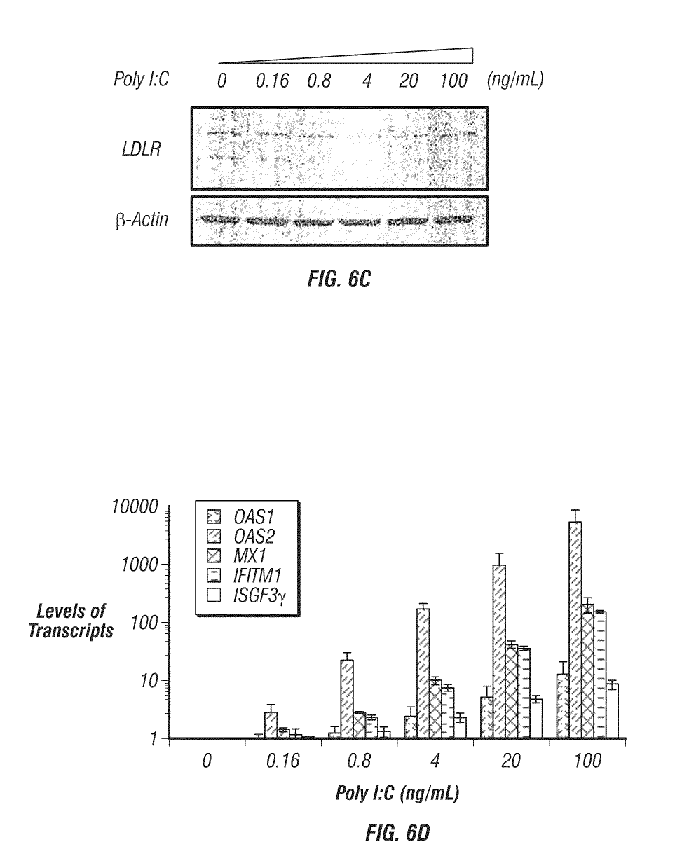

Effect of agRNAs on Expression of Interferon Responsive Genes. Some small RNAs can induce off-target effects through induction of the interferon response (Hornung et al., 2005; Birmingham et al., 2006). This potential activity is important for studies with LDLR because some cytokines have been reported to promote enhanced LDLR expression and increased LDL binding in cells (Stopeck, et al., 1993; Ruan et al., 1998). To investigate involvement of interferon response to LDLR activation by agRNAs, the inventors evaluated expression of interferon responsive genes by qRT-PCR after transfection of unmodified or modified agRNAs, LDLR-24(U/U), LDLR-24(U/O), LDLR-24(F/U), LDLR-28(U/U), LDLR-28(U/O), and LDLR-28(F/U). These agRNAs yielded only small changes for levels of interferon-responsive gene expression including OAST, OAS2, MX1, IFITM1, and ISGF3γ (FIGS. 6A-B). Addition of polyinosinic-polycytidylic acid (poly I:C), a potent inducer of interferon response, substantially increases interferon responsive gene expression, but did not upregulate LDLR expression at any concentrations tested in HepG2 cells (FIGS. 6C-D). Taken together, these data suggest that gene activation by LDLR-24, LDLR-28, and their chemically modified variants is not due to induction of interferon-responsive genes.

Addition of agRNAs and 25-Hydroxycholesterol. The membrane-bound transcription factor SREBP binds to a sterol regulatory element within the LDLR promoter and triggers increased transcription of the LDLR gene (Brown and Goldstein, 1997). 25-hydroxycholesterol represses LDLR expression by inhibiting the processing step that yields active NH₂-terminal fragments of SREBP (Adams et al., 2004). To determine whether addition of agRNAs might override this repression and permit enhanced LDLR expression, the inventors added agRNA LDLR-24(U/U) or LDLR-28(U/U) in combination with 25-hydroxycholesterol.

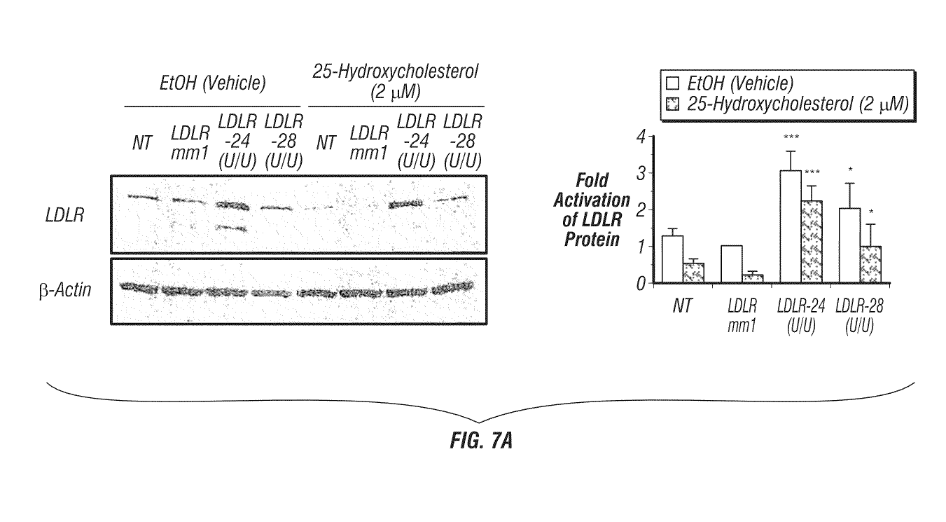

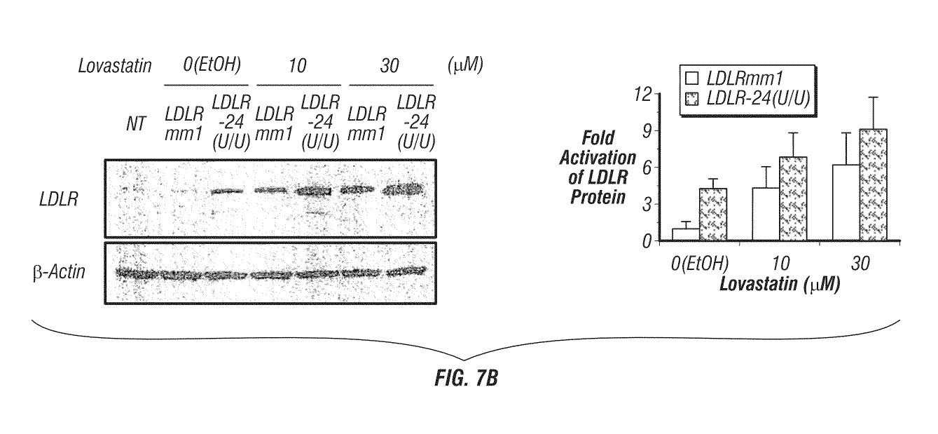

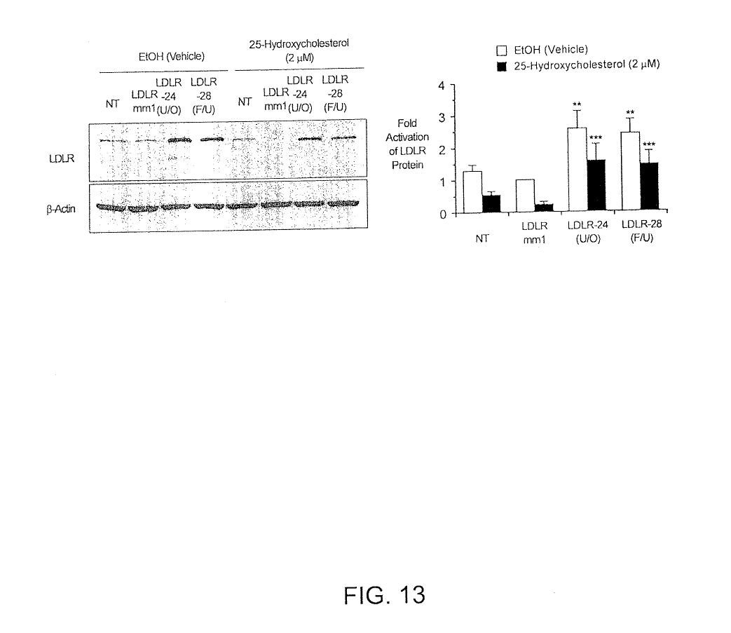

We observed that LDLR-24(U/U) activated LDLR expression regardless of whether 25-hydroxycholesterol was present. Because treatment with 25-hydroxycholesterol lowers baseline LDLR expression, the relative activation by anti-LDLR agRNAs increased from 2-3 fold in cells grown under standard conditions to 4-9 fold (FIG. 7A). This result has practical importance because, by suppressing basal expression, agRNA-mediated activation can be observed more clearly. Screening for activating agRNAs using cells treated to reduce basal levels of gene activation may be a useful strategy for more rapidly identifying the most promising agRNAs. Similar increases of LDLR expression were achieved using chemically modified agRNAs LDLR-24(U/O) and LDLR-28(F/U) in the presence of 25-hydroxycholesterol (FIG. 13).

Addition of agRNAs and Lovastatin. Lovastatin is an HMG-CoA reductase inhibitor whose administration leads to increased levels of LDLR (Alberts, 1988). It is a US Food and Drug Administration (FDA)-approved drug for lowering plasma LDL-c and comparing its activity with agRNAs offers a useful metric for evaluating the potential of agRNA-mediated modulation of LDLR expression. Addition of agRNA LDLR-24(U/U) or lovastatin alone led to an similar increase in expression of LDLR (FIG. 7B). When the inventors combined lovastatin and LDLR-24(U/U) in HepG2 cells, LDLR levels were significantly greater than when either agent was added individually, suggesting that the activities of lovastatin and anti-LDLR agRNAs are additive.

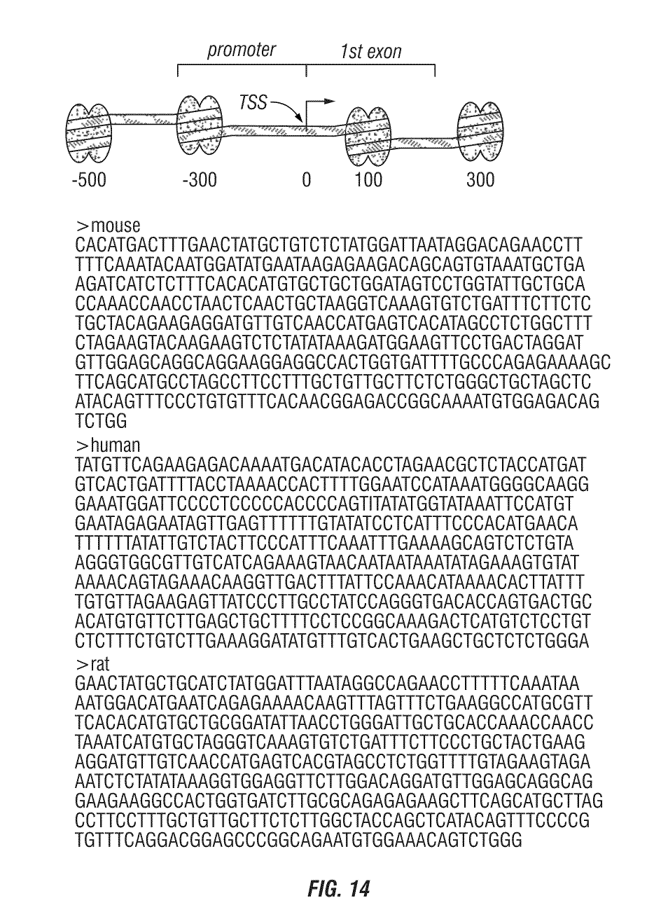

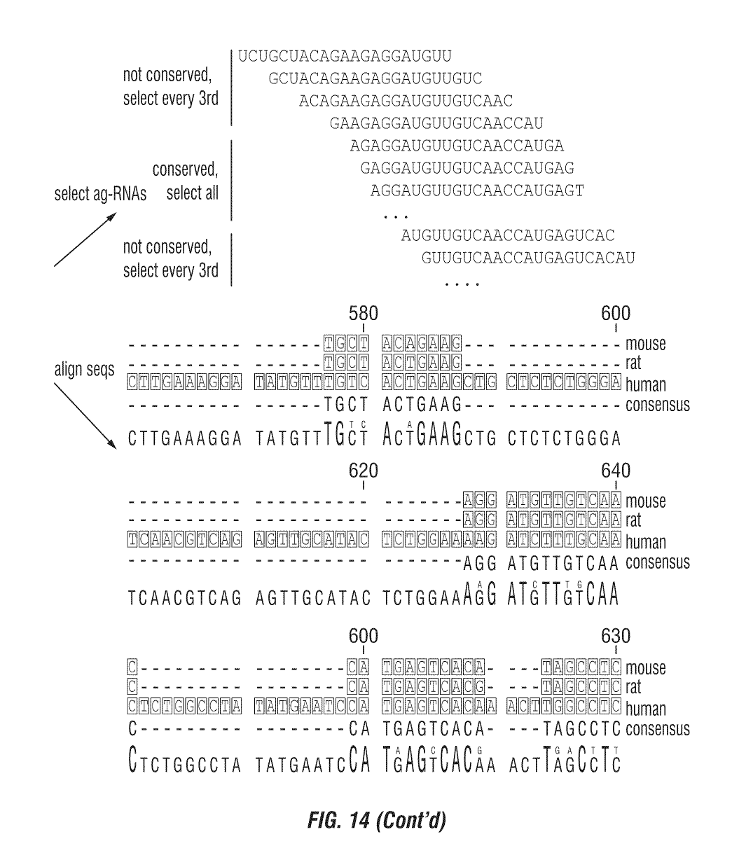

Design criteria to select RNAs-targeting the LDL-R promoters. Promoters are not conserved across species. Therefore, to improve the design of agRNAs, the inventors extracted the genomic LDL-R sequences from mouse, rat and human from 200 nucleotides upstream of the transcription start site (TSS) to the beginning of the first intron. Every 19-mer was extracted from the mouse sequence, and it was determined whether each of these 19-mers had a perfect match to either rat or human sequences. For all sequences that did not have a match, one sequence was outputted at every 3rd nucleotide sequence. Then, duplexes were selected starting from 100 nucleotides upstream of the transcription start site (TSS) in mouse and ending 10 nts upstream of the TSS (−9→−99). No off-target scoring was performed. FIG. 14 illustrates this process.

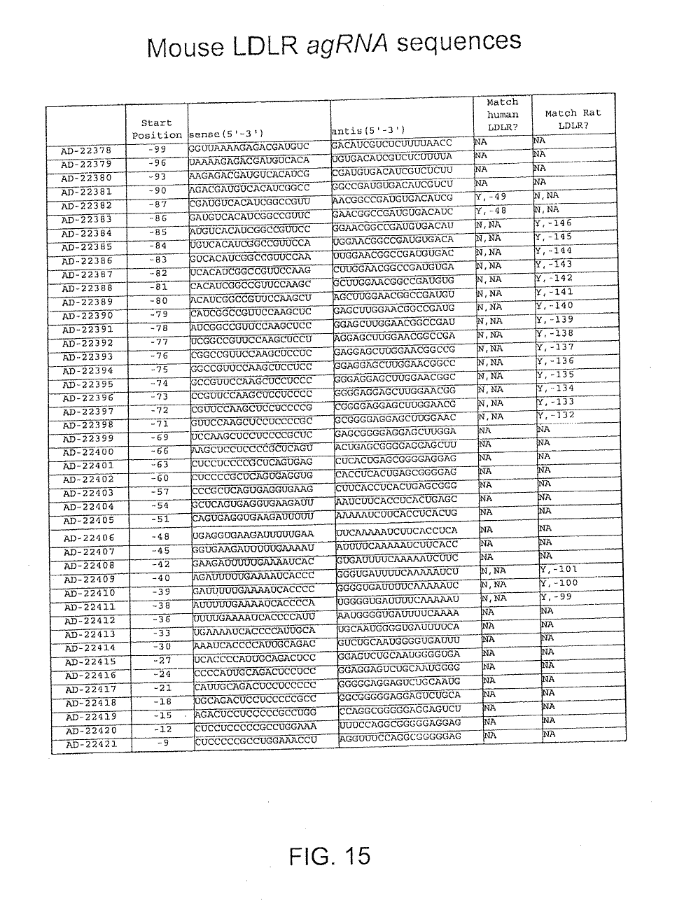

The set was much smaller than with siRNA selection, with 44 agRNA sequences: 27 mouse-specific, 2 cross-reactive mouse/human, and 15 cross-reactive mouse/rat (FIG. 15). The sequences were synthesized with unmodified bases as 21-mers (duplex) with two dTdT double overhangs.

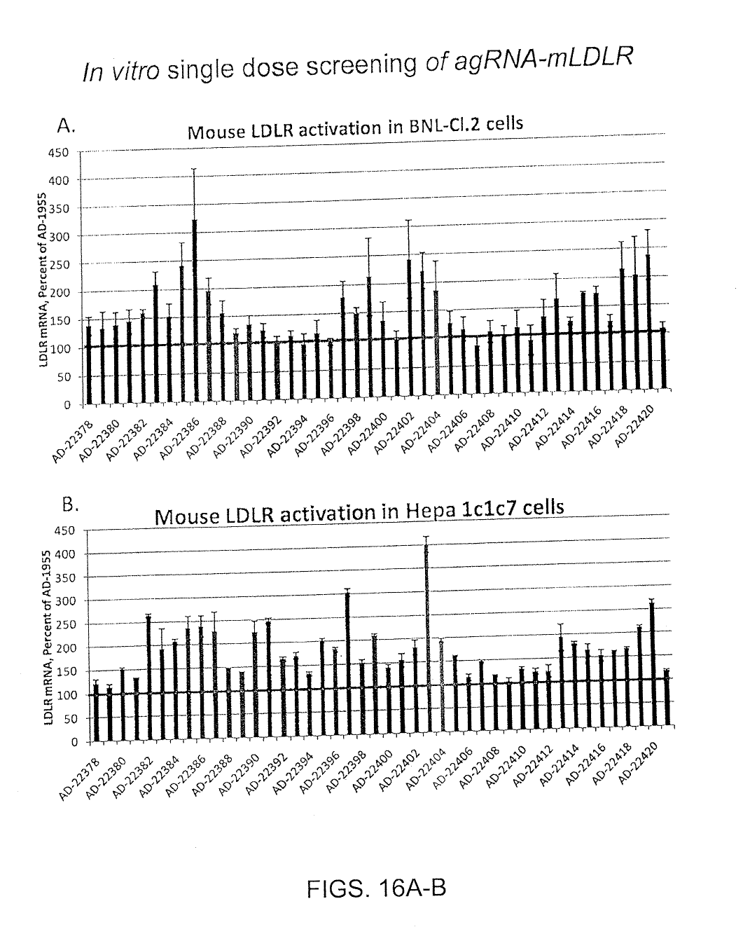

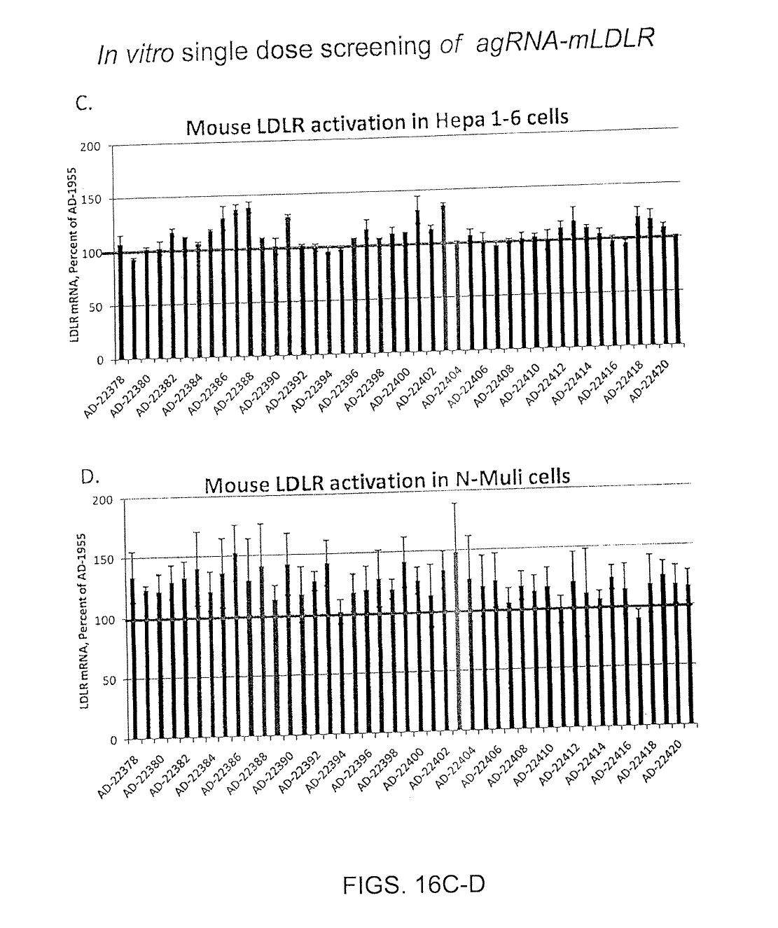

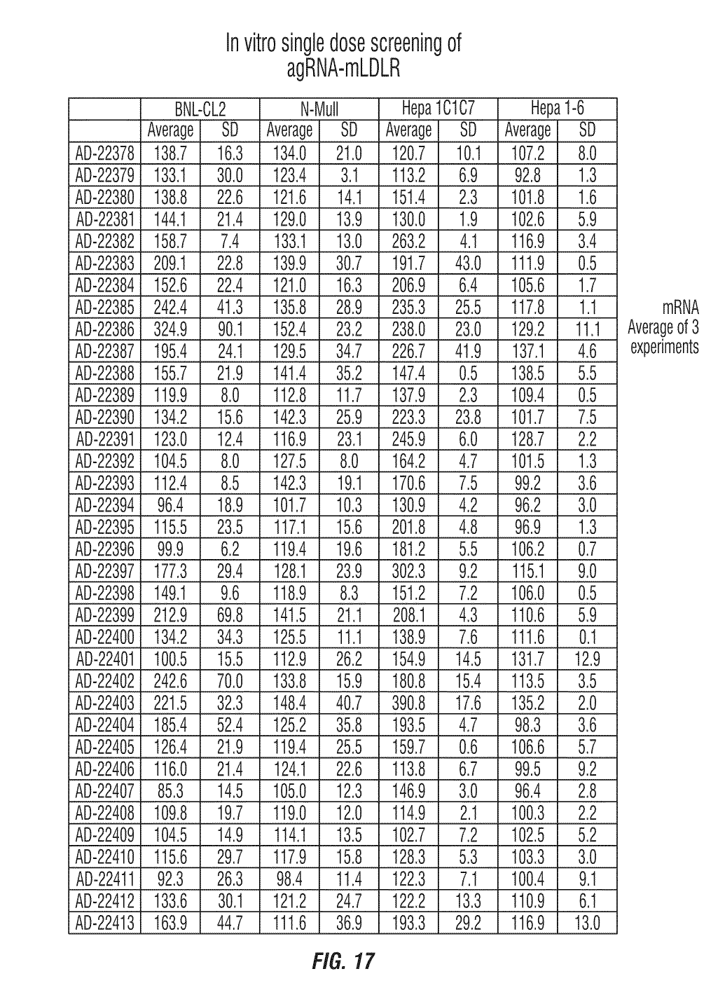

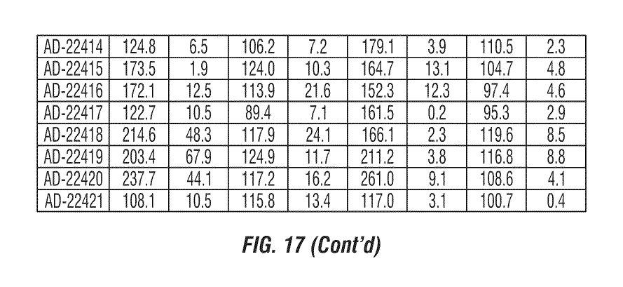

In vitro single dose screening. Four murine cell lines were used—BNL-Cl.2, Hepa 1C1C7, Hepa 1-6, and N-Muli—to assess the activation in relevant cell lines. Mouse cell lines were cultured using standard conditions in DMEM with 10% FBS. Generally, a non-specific duplex AD-1955 or BlockIT was used as non-specific controls. In certain cells, PBS or mock-transfected controls were used. Screens were performed for mRNA up-regulation of the target gene in the identified cell lines using single dose (50 nM or 25 nM) of RNA duplex formulated in OptiMEM (Invitrogen) in 96-well plates. Each well of the 96-well plate contained final values of 0.2 μl Lipofectamine RNAiMax, 25 nM or 50 nM of the duplex, and 12,000 cells in 100 μl. The media was changed 24 hours after transfection. All plates were lysed and prepared for measurement of mRNA levels using the branched DNA (bDNA) method on 72 hrs after transfection. The lysates were diluted 2:3 (i.e., 100 μL buffer added to 200 μl sample) for the mouse LDL-R probe and 1:10 for the mouse GAPDH probe. Two to four biological replicates were transfected for each duplex and cell type. mRNA levels were quantified by branched DNA assay using the Quantigene 2.0 bDNA kit (Panomics/Affymetrix) performed essentially as described by the manufacturer. Briefly, samples in 96-well plates were lysed in a solution containing two parts nuclease-free water and one part lysis mixture. Proteinase K stock (50 μg/ml) was added to a final volume of 10 μl Proteinase K per ml prepared solution. The plates were then incubated at 55° C. for 60 minutes. After incubation, diluted lysates were added to bDNA plates with blocking buffer and probes targeting human/mouse GAPDH and human/mouse LDL-R. The plates were incubated overnight at 55° C. On the next day, the plates were removed to room temperature and washed using an automated washer. Wells were washed by three additions and removal of 300 μL wash solution per well. The following steps consisted of three one-hour incubations in preamplifier, amplifier, and labeling probe respectively. The first two incubations were performed at 55° C., while the labeling probe incubation was at 50° C. Between each step was a set of washes as described above. After a final series of washes, substrate was added. Luminescence was measured in the wells using a spectrophotometer and an integration time of 200 milliseconds. Background luminescence was determined by omission of sample, and was subtracted from all data. The effects of various ag-RNA compositions on the mRNA levels of LDL-R in the four cell types are shown on FIGS. 16A-D (BNL-Cl.2 cells in FIG. 16A, Hepa 1C1C7 in FIG. 16B, Hepa 1-6 in FIG. 16C, and N-Muli in FIG. 16D). The data for the various cell lines (average for three experiments) are shown in FIG. 14. Data are expressed as percent of AD-1955 or BlockIT (non-specific controls).

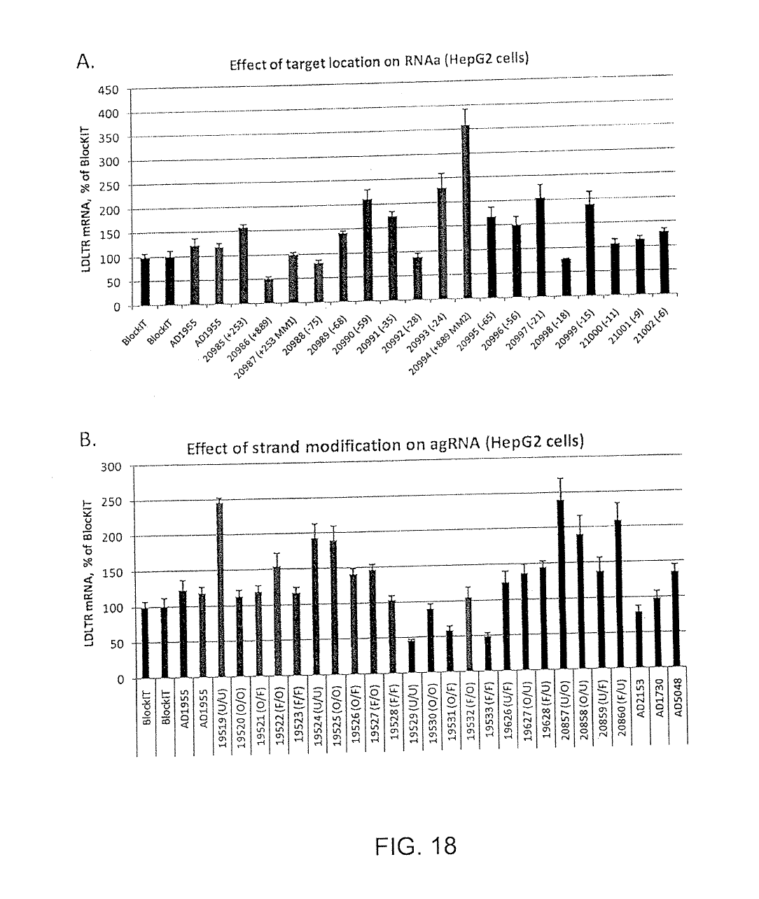

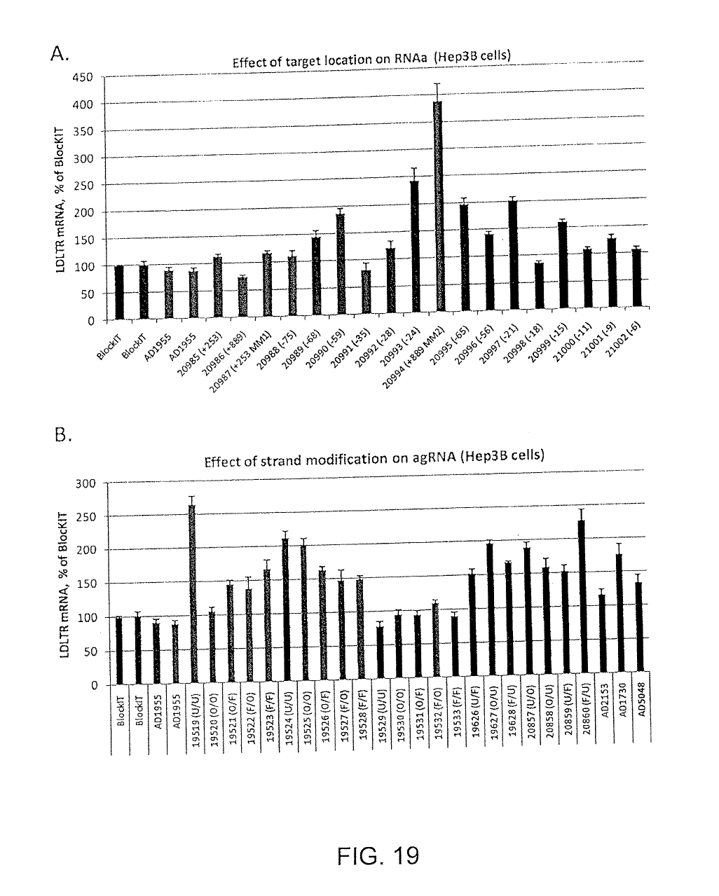

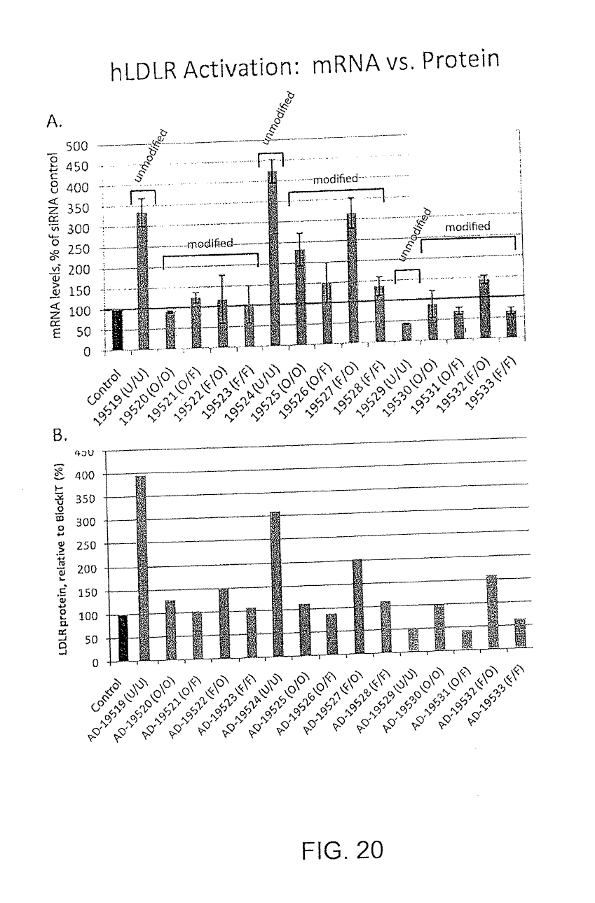

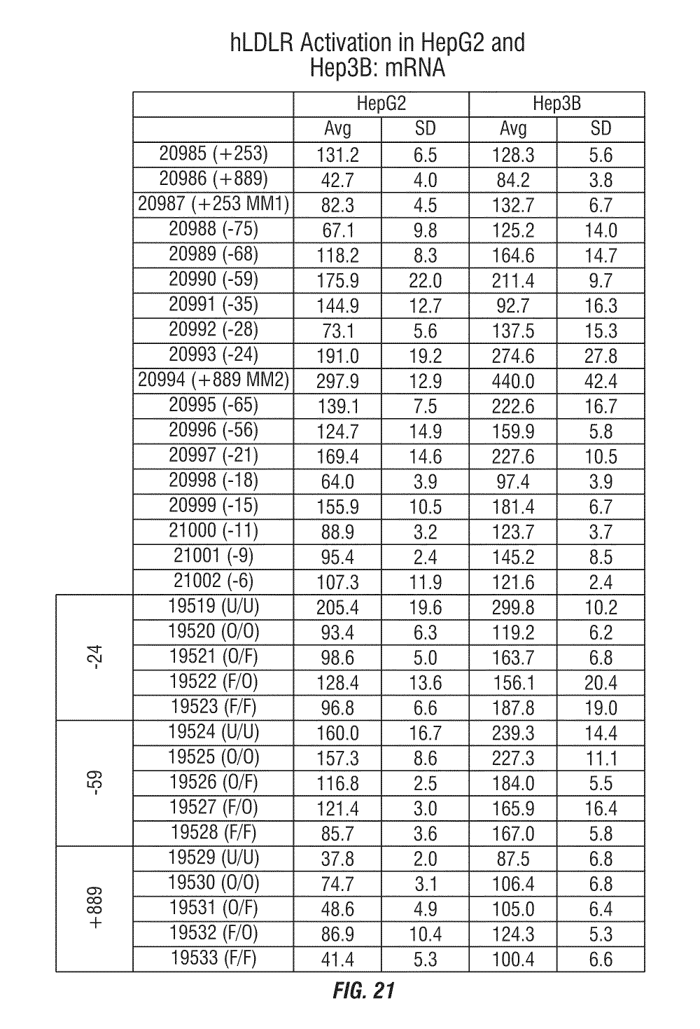

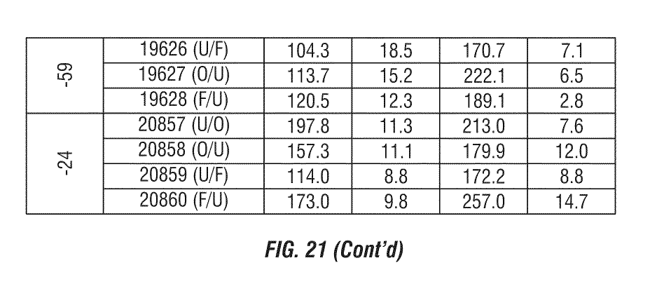

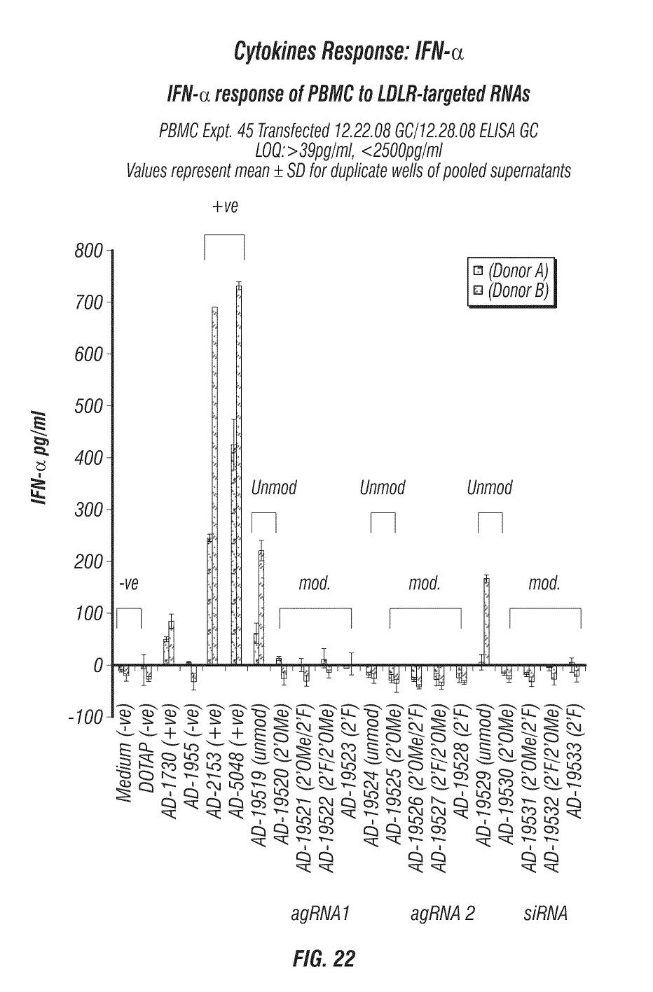

Activation of human LDR by unmodified and modified agRNA in HepG2 and Hep3B cells. HepG2 were cultured in MEM with Earle's salts (Invitrogen), 10% FBS, 2 mM glutamine, 0.1 mM MEM non-essential amino acids, 1 mM sodium pyruvate, and 1.5 g/L sodium bicarbonate. Hep3B cells were cultured in EMEM with 10% FBS and 5% Glutamax. HepG2 and Hep3B cells were (reverse) transfected using 50 nM or 25 nM agRNA and approximately 12,000-20,000 in 100 μl per well. Modified and unmodified duplexes targeting various regions of the human LDL-R promoter were tested. bDNA assays were performed essentially as described above to measure mRNA levels; samples were collected three days after transfection. The activation of LDL-R mRNA in HepG2 cells transfected with duplexes targeting various regions of the hLDL-R promoter are shown in FIG. 18A; the effect of strand modification on activation of LDL-R mRNA in HepG2 cells is shown in FIG. 18B. Corresponding experiments were performed in Hep3B cells, the results of which are shown in FIG. 19A and FIG. 19B. To determine whether there is a differential effect of the ag-RNA on LDL-R mRNA and protein levels, LDL-R mRNA (by bDNA assay; FIG. 20A) and protein (by Western blotting; FIG. 20B) were measured; strong correlation between mRNA and protein levels were observed. The effect of the various duplexes on Hep3B and HepG2 cells is summarized in FIG. 21.

In vitro PBMC Assay to examine cytokine stimulation of duplexes. To examine the ability of duplexes to stimulate interferon alpha (IFNα) or tumor necrosis factor alpha (TNFα), human peripheral blood mononuclear cells (hPBMCs) were isolated from concentrated fractions of leukocytes (buffy coats). Buffy coats were diluted 1:1 in PBS, added to a tube of Histopaque (Sigma, St. Louis, Mo.) and centrifuged for 20 minutes at 2200 rpm to allow fractionation. White blood cells were collected, washed in PBS, followed by centrifugation. Cells were resuspended in RPMI 1640 culture medium (Invitrogen) supplemented with 10% fetal calf serum, IL-3 (10 ng/ml) (Sigma) and phytohemagglutinin-P(PHA-P) (5 μg/ml) (Sigma) for IFNα assay, or with no additive for TNFα assay at a concentration of 1×10⁶ cells/ml, seeded onto 96-well plates and incubated at 37° C., 5% CO₂. Control oligonucleotides siRNA AL-DP-5048 duplex:

| | |
|---|---|
| 5′-GUCAUCACACUGAAUACCAAU-3′ | (SEQ ID NO: 315) |
| and | |
| 3′-CACAGUAGUGUGACUUAUGGUUA-5′; | (SEQ ID NO: 316) |
| siRNA AL-DP-7296 duplex: | |
| 5′-CUACACAAAUCAGCGAUUUCCAUGU-3′ | (SEQ ID NO: 317) |
| and | |
| 3′-GAUGUGUUUAGUCGCUAAAGGUACA-5′. | (SEQ ID NO: 318) |

Cells in culture were combined with either 500 nM oligonucleotide, pre-diluted in OptiMEM (Invitrogen), or 133 nM oligonucleotide pre-diluted in OptiMEM and Geneporter, GP2 transfection reagent (Genlantis, San Diego, Calif.) for IFNα assay or N-[1-(2,3-Dioleoyloxy)propyl]-N,N,N-trimethylammonium methylsulfate (DOTAP) (Roche, Switzerland) for TNFα assay and incubated at 37° C. for 24 hrs. IFNα and TNFα were measured using the Bender MedSystems (Vienna, Austria) instant ELISA kit according to manufacturer's instruction.

The foregoing description and examples are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### VI. References

The following references, to the extent that they provide exemplary procedural or other details supplementary to those set forth herein, are specifically incorporated herein by reference:
U.S. Pat. No. 4,981,957
U.S. Pat. No. 5,118,800
U.S. Pat. No. 5,279,721
U.S. Pat. No. 5,319,080
U.S. Pat. No. 5,359,044
U.S. Pat. No. 5,393,878
U.S. Pat. No. 5,446,137
U.S. Pat. No. 5,466,786
U.S. Pat. No. 5,514,785
U.S. Pat. No. 5,519,134
U.S. Pat. No. 5,567,811
U.S. Pat. No. 5,576,427
U.S. Pat. No. 5,591,722
U.S. Pat. No. 5,595,760
U.S. Pat. No. 5,597,909
U.S. Pat. No. 5,610,300
U.S. Pat. No. 5,627,053
U.S. Pat. No. 5,639,873
U.S. Pat. No. 5,646,265
U.S. Pat. No. 5,658,873
U.S. Pat. No. 5,670,633
U.S. Pat. No. 5,672,659
U.S. Pat. No. 5,700,920
U.S. Pat. No. 5,757,994
U.S. Pat. No. 5,788,166
U.S. Pat. No. 5,792,747
U.S. Pat. No. 5,838,002
U.S. Pat. No. 5,986,258
U.S. Pat. No. 6,268,490
U.S. Pat. No. 6,525,191
U.S. Pat. No. 6,531,584
U.S. Pat. No. 6,600,032
U.S. Pat. No. 6,670,461
U.S. Pat. No. 6,770,748
U.S. Pat. No. 6,794,499
U.S. Pat. No. 7,034,133
U.S. Pat. No. 7,053,207
U.S. Patent RE 35,413
U.S. Patent Publn. 2003/0082807
U.S. Patent Publn. 2003/0207841
U.S. Patent Publn. 2004/0014959
U.S. Patent Publn. 2004/0143114
U.S. Patent Publn. 2004/0171570
U.S. Patent Publn. 2004/0219565
U.S. Patent Publn. 2007/0213292
U.S. Patent Publn. 2007/0287831
U.S. Patent Publn. 2008/0015162
U.S. Patent Publn. 2008/0032945
Adams et al., J. Biol. Chem., 279:52772-52780, 2004.
Akinc et al., Nat. Biotechnol., 26(5):561-9, 2008.
Alberts, Am. J. Cardiol., 62:10J-15J, 1988.
Allerson et al., J. Med. Chem., 48:901-904, 2005.
Bahr et al., J. Mass Spectrom., 32:1111-1116, 1997.
Bentzley et al., Anal Chem., 68(13):2141-2146, 1996.
Birmingham et al., Nat. Meth., 3:199-204, 2006.
Braasch et al., Methods, 23(2):97-107, 2001.
Bramsen et al., Nucl. Acids Res., 35:5886-5897, 2007.
Brown and Goldstein, Cell, 89:331-340, 1997.
Bucknall et al., J. Am. Soc. Mass Spectrom., 13(9):1015-1027, 2002.
Caprioli et al., Anal. Chem., 69:4751, 1997.
Chaurand et al., Anal Chem., 71(23):5263-5270, 1999.
Chen et al., Biomed Chromatogr., 15(8):518-24, 2001.
Choung et al., Biochem. Biophys. Res. Commun., 342:919-927, 2006.
Chu et al., Nucleic Acid Res., 2010 (IN PRESS).
De Paula et al., RNA, 13:431-456, 2007.
Desiderio et al., J. Mass Spectrom., 35(6):725-733, 2000.
Desiderio et al., Methods Mol. Biol., 61:57-65, 1996.
Duncan et al., Rapid Commun. Mass Spectrom., 7(12):1090-1094, 1993.
Elayadi et al., Nucleic Acids Res., 29(8):1683-9, 2001.
Elmén et al., Nature, 452(7189):896-9, 2008.
Esau et al. Cell Metab., 3(2):87-98, 2006.
Faulstich et al., Anal. Chem., 69(21):4349-4353, 1997.
Fenn et al., Science, 246(4926):64-71, 1989.
Frieden et al., Nucle. Nucleo. Nucleic Acids, 22(5-8):1041-3, 2003.
Gobom et al., Anal. Chem., 72(14):3320-3326, 2000.
Haeberli et al., Nucleic Acids Res., 33:3965-3975, 2005.
Hall et al., Clin. Cancer Res., 10(23):7852-7859, 2004.
Hall et al., Nucleic Acids Res., 34:2773-2781, 2006.
Han et al., Proc. Natl. Acad. Sci. USA, 104:12422-12427, 2007.
Hassani et al., J. Gene Med., 7:198-207, 2004.
Horak et al., Rapid Commun. Mass Spectrom., 15(4):241-248, 2001.
Hornung et al., Nat. Med., 11:263-270, 2005.
Hoshika et al., Nucleic Acids Res., 32:3815-3825, 2004.
Ishiwata et al., Chem. Phare. Bull., 43:1005, 1995.
Jackson et al., RNA, 12:1197-1205, 2006.
Janowski et al., Nat. Chem. Biol., 1:216, 2005.
Janowski et al., Nat. Chem. Biol., 3:166, 2007.
Janowski et al., Nat. Struct. Mol. Biol., 13:787, 2006.
Jespersen et al., Anal Chem., 71(3):660-666, 1999.
Jiang et al., Biochem. Pharmacol., 59:763-772, 2000.
Judge and MacLachlan, Hum. Gene Ther., 19:111-124, 2008.
Kabarle et al., Anal. Chem. 65(20):972A-986A, 1993.
Kaihatsu et al., Biochem., 42(47):13996-4003, 2003.
Kaihatsu et al., Chem. Biol., 11:749-758, 2004.
Kanazawa et al., Biol. Pharm. Bull., 22(4):339-346, 1999.
Kaur et al., Biochemistry, 45(23):7347-7355, 2006.
Kazmaier et al., Anesthesiology, 89(4):831-817, 1998.
Kim et al., Nat. Struct. Mol. Biol., 13:793, 2006.
Koshkin et al., Tetrahedron, 54:3607-3630, 1998.
Krützfeldt et al., Nature, 438:685-689, 2005.
Kumar et al., Bioorg. Med. Chem. Lett., 8:2219-2222, 1998.
Lasic et al., Chem. Rev. 95:2601, 1995.
Lasic et al., Science, 267:1275, 1995.
Li et al., Proc. Natl. Acad. Sci. USA, 102:19231-19236, 2005.
Li et al., Proc. Natl. Acad. Sci. USA, 103:17337, 2006.
Liu et al. Science, 305:1437-1441, 2004.
Liu et al., J. Biol. Chem., 42:24864, 1995.
Lovelace et al., J. Chromatogr., 562(1-2):573-584, 1991.
Lynn et al., J. Mol. Evol., 48(5):605-614, 1999.
Marie et al., Anal. Chem., 72(20):5106-5114, 2000
McMahon et al., Life Sci., 71(3):325-3, 2002.
Meister et al. Mol. Cell, 15:185-197, 2004.
Miketova et al., Mol. Biotechnol., 8(3):249-253, 1997.
Mirgorodskaya et al., Rapid Commun. Mass Spectrom., 14(14):1226-1232, 2000.
Morimoto et al., J. Immunol., 176:7657-7665, 2006.
Morita et al., Nucleic Acids Res. Suppl., (2):99-100, 2002
Morris et al., PLoS Genetics, 4:e1000258, 2008.
Morris et al., Science, 305:1289-1292, 2004.
Muddiman et al., Fres. J. Anal. Chem., 354:103, 1996.
Napoli et al., EMBO J., 28:1708-1719, 2009.
Nelson et al., Rapid Commun. Mass Spectrom., 8(8):627-631, 1994.
Nguyen et al., J. Chromatogr. A., 705(1):21-45, 1995.
Nielsen et al., Science, 254:1497-1500, 1991.
Paroo and Corey, Trends Biotechnol., 22(8):390-4, 2004.
PCT Appln. WO 2005/021570
PCT Appln. WO 2005/121371
PCT Appln. WO 2005115481
PCT Appln. WO 94/14226
PCT Appln. WO 96/10390
PCT Appln. WO 96/10391
PCT Appln. WO 96/10392
PCT Appln. WO 98/39352
PCT Appln. WO 99/14226
Pignon et al., Hum. Mutat., 3: 126-132, 1994.
Prakash et al., J. Med. Chem., 48:4247-4253, 2005.
Rand et al. Proc. Natl. Acad. Sci. USA, 101:14385-14389, 2004.
Rand et al., Cell, 123:621-629, 2005.
Remington's Pharmaceutical Sciences, Mack Publishing Co., 1985.
Remington's Pharmaceutical Sciences, 18th Ed. Mack Printing Company, 1289-1329, 1990.
Roepstorff, In: MALDI-TOF Mass Spectrometry Protein Chemistry, Jolles and Jornvall (Eds.), 1-220, 2000.
Ruan et al., Nephrol. Dial. Transplant., 13:1391-1397, 1998.
Schwartz et al., Nat. Struct. Mol. Biol., 15:842, 2008.
Singh et al., Chem. Commun., 4:455-456, 1998
Singh et al., J. Org. Chem., 63:10035-10039, 1998
Siomi and Siomi, Nature, 457:396-404, 2009.
Soutschek et al., Nature, 432(7014):173-178, 2004.
Stoeckli et al., Nat. Med., 7(4):493-496, 2001.
Stopeck, et al., J. Biol. Chem., 268:17489-17494, 1993.
Summerton and Weller, Antisense Nucleic Acid Drug Dev.;7(3):187-95, 1997.
Takach et al., J. Protein Chem., 16:363, 1997.
The Science and Practice of Pharmacy, 2003.
Ting et al., Nat. Genet., 37:906, 2005.
Tyler et al., FEBS Lett., 421:280, 1999.
Tyler et al., Proc. Natl. Acad. Sci. USA, 96:7053-7058, 1999.
Urban-Klein, Gene Ther., 12:461-6, 2005.
Villanueva et al., Genes Dev., 13:3160-3169, 1999.
Wahlestedt et al., Proc. Natl. Acad. Sci. USA, 97:5633-5638, 2000
Watts et al., Drug Discov. Today, 13:842-855, 2008.
Watts et al., Nucleic Acids Res., 38(15):5242-59, 2010.
Wittmann et al., Biotechnol. Bioeng., 72:642, 2001.
Wu et al., Anal. Biochem., 263(2):129-38, 1998.
Wu et al., Biochem. Biophys. Res. Commun., 233(1):221-226, 1997.
Wu et al., Rapid Commun Mass Spectrom., 14(9):756-64, 2000.
Yang et al., J. Agric. Food Chem., 48(9):3990-6, 2000.
Yue et al., Nat. Chem. Biol., 6:621-629, 2010.
Zhong et al., Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

1. A method of modulating expression of low density lipoprotein receptor (LDL-R) in a cell comprising contacting said cell with a first double-stranded RNA complementary to a portion of an LDL-R promoter.

2. The method of claim 1, wherein the double-stranded RNA increases LDL-R expression.

3. The method of claim 1, wherein the double-stranded RNA decreases LDL-R expression.

4. The method of claim 1, wherein the double-stranded RNA targets a Repeat 2 region, a Repeat 3 region or both.

5. The method of claim 1, wherein the double-stranded RNA targets a sterol-independent regulatory element.

6. The method of claim 1, wherein the double-stranded RNA is complementary a region within bases −1 to −200, relative to the transcription start site, of the LDL-R gene.

7. (canceled)

8. The method of claim 1, wherein the double-stranded RNA contains one or more modified nucelosides.

9. (canceled)

10. (canceled)

11. The method of claim 8, wherein one strand of the double-stranded RNA contains one or more modified nucelosides, and the other strand does not contain a modified nuceloside.

12. (canceled)

13. (canceled)

14. The method of claim 1, wherein said cell is located in situ in a host, and the contacting step is effected by administering to the host an effective amount of the double-stranded RNA.

15. (canceled)

16. The method of claim 1, further comprising detecting a change in the expression of LDL-R.

17. The method of claim 16, wherein detecting comprises inferring a change in the expression from a physiologic change in the cell.

18. The method of claim 17, wherein the cell is located in situ in a host and detecting comprises inferring a change in the expression from a physiologic change in the host.

19. The method of claim 16, wherein detecting comprises one or more of Northern blot, PCR, immunohistochemistry, Western blot or ELISA.

20. The method of claim 1, wherein the RNA further comprises one or more deoxyribonucleotides.

21. The method of claim 20, wherein the RNA comprises a dTdT dinucleotide overhang of each strand.

22. The method of claim 20, wherein the RNA comprises at least one phosphorothioate linkage in each strand.

23.-25. (canceled)

26. The method of claim 1, wherein said double-stranded RNA is formulated in a lipid vehicle.

27. A method of reducing circulating low density lipoprotein in a subject comprising administering to said subject a first double-stranded RNA complementary to a portion of an LDL-R promoter.

28. The method of claim 27, wherein the subject suffers from hypercholesterolemia, atherolsclerosis and/or coronary heart disease.

29.-35. (canceled)

36. The method of claim 27, wherein said double-stranded RNA is formulated in a lipid vehicle.

37. A pharmaceutical formulation comprising (a) a double-stranded RNA complementary to a portion of a low density lipoprotein receptor (LDL-R) promoter, and (b) a pharmaceutically acceptable buffer, carrier or diluent.

38.-43. (canceled)
